(19) United States
(12) Patent Application Publication
(10) Pub. No.: US 2021/0163443 A1
(43) Pub. Date: Jun. 3, 2021

(54) Febrifugine Derivatives

(71) Applicant: BRISTOL-MYERS SQUIBB COMPANY, Princeton, NJ (US)

(72) Inventors: Pravin Sudhakar Shirude, Bangalore (IN); Satheesh Kesavan Nair, Bangalore (IN); Ramesh Kumar Sistla, Bangalore (IN); Ashit K. Ganguly, Belmont, MA (US); Ramakanth Sarabu, Towaco, NJ (US)

(21) Appl. No.: 17/108,024

(22) Filed: Dec. 1, 2020

Related U.S. Application Data
(60) Provisional application No. 62/942,355, filed on Dec. 2, 2019.

(51) Int. Cl. C07D 401/06; C07D 401/14; C07D 405/14; C07D 417/14

(52) U.S. Cl. CPC C07D 401/06; C07D 417/14; C07D 405/14; C07D 401/14

(57) ABSTRACT

Disclosed are compounds of Formula I, and pharmaceutically acceptable salts thereof:

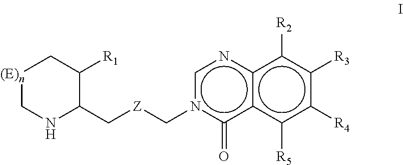

Also disclosed are pharmaceutical compositions thereof as well as methods for using the compounds in treating a variety of diseases such as, for example, autoimmune diseases, dry eye syndrome, fibrosis, scar formation, angiogenesis, ischemic damage, inflammatory diseases, cancers, musculoskeletal diseases, cardiovascular diseases, transplant rejection, multiple sclerosis, systemic sclerosis and neurodegenerative diseases.

### CROSS REFERENCE

This application claims the benefit of U.S. Provisional Application Ser. No. 62/942,355 filed Dec. 2, 2019, which is incorporated herein in its entirety.

### FIELD OF THE INVENTION

The present invention generally relates to compounds that are derivatives of febrifugine, methods of making the compounds, pharmaceutical compositions comprising the compounds and methods of using the compounds to treat diseases.

### BACKGROUND OF THE INVENTION

Febrifugine is a natural product extracted from the roots of the hydrangea Dichroa febrifuga. Dichroa febrifuga is one of the “fifty fundamental herbs” of traditional Chinese medicine, originally used as an anti-malarial remedy (Jiang et al., Antimicrob. Agents Chemother. (2005) 49: 1169-1176).

Halofuginone is a halogenated derivative of febrifugine. Halofuginone (7-bromo-6-chloro-3-[3-(3-hydroxy-2-piperidinyl)-2-oxopropyl]-4(3H)-quinazolinone), and halofuginone derivatives are described in U.S. Pat. No. 2,694,711. Halofuginone is disclosed as having anti-fibrotic properties in vivo (Pines, et al., Biol. Blood Marrow Transplant (2003) 9: 417-425; U.S. Pat. No. 6,028,075). However, halofuginone has shown some toxicity in humans, such as nausea, vomiting, and fatigue, and possibly bleeding complications (de Jonge et al., Eur. J. Cancer (2006) 42: 1768-1774).

Patent applications WO2010019210, published Feb. 18, 2010 and WO2013106702, published Jul. 18, 2013 disclose derivatives of halofuginone. In the Abstract of WO2013106702, it is disclosed that:

The present invention provides halofuginol, and derivatives and salts thereof, including diasteromerically enriched compositions thereof. The invention also provides pharmaceutical and cosmetic compositions thereof as well as methods for using halofuginol and derivatives thereof in treating chronic inflammatory diseases, autoimmune diseases, dry eye syndrome, fibrosis, scar formation, angiogenesis, viral infections, malaria, ischemic damage, transplant rejection, neurodegenerative diseases, T-cell neoplasms, and cosmetic conditions.

Despite the recognition of the potential medical uses for febrifugine and discovery of halofuginone, halofuginol and other compounds, new derivatives of febrifugine are desired that may provide benefits to patients such as, for example, enhanced activity and less side effects.

### SUMMARY OF THE INVENTION

The present invention is directed to derivatives of febrifugine, methods of making the compounds, pharmaceutical compositions comprising the compounds and methods of using the compounds to treat diseases. By virtue of the present invention, it may now be possible to provide more effective treatment to patients with the compounds of the present invention. Patients may experience an improved response in one or more areas including, for example, increased efficacy, reduced side effects, and enhanced quality of life.

In one aspect of the invention, there is provided a compound of Formula I

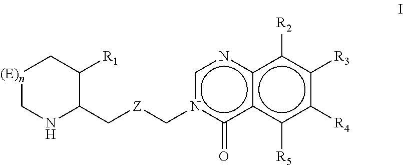

where:
E is CH₂, where n is 0 or 1;

### Z is C═O or CH—OH;

R₁ is —OH or —OCOC(CH₃)₃;
R₂ is hydrogen, —CH₃ or —CH₂CH₃;
R₃ is hydrogen, —CH₃, or —OCH₃;
R₄ is selected from hydrogen, halogen, —CH₂CH₃, —CHCH₃CH₃, —OCF₃, —C≡N, substituted or unsubstituted aryl, substituted or unsubstituted heteroaryl, cycloalkyl, 5 or 6 membered substituted or unsubstituted saturated or unsaturated heterocycle, —SO₂CH₃, or —CF₃;
R₅ is selected from hydrogen, halogen, —CF₃, substituted or unsubstituted aryl, or substituted or unsubstituted heteroaryl, or cycloalkyl;
or a pharmaceutically acceptable salt thereof.

In an aspect of the invention, R₁ is —OH. In an aspect of the invention, R₂ is —CH₃. In an aspect of the invention, R₃ is hydrogen. In an aspect of the invention, R₂ is hydrogen. R₃ is —CH₃. In an aspect of the invention, R₂, R₃ and R₄ are hydrogen. In an aspect of the invention, when n=0, Z is ═O, and R₂ and R₅ are H, then R₁ is not —OH in the compound of Formula 1. In an aspect of the invention, R₄ is a substituted or unsubstituted aryl. In an aspect of the invention, R₄ is selected from aryl substituted with one or more halogen or —CF₃, or a 5 or 6 membered heterocycle. In an aspect of the invention, R₄ is selected from pyrrolidine, oxane, pyridine, thiazole or pryazole. In an aspect of the invention, R₅ is selected from aryl substituted with one or more halogens, —CF₃, or a heteroaryl. In an aspect of the invention, R₅ is pyridine. In an aspect of the invention, R₃ is a substituent other than bromo. In an aspect of the invention, R₄ is a substituent other than chloro.

In one aspect of the invention, there is provided a compound of Formula II

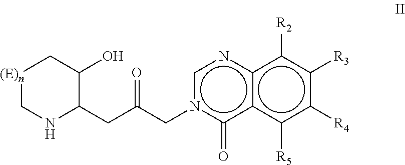

where:
E is CH₂, where n is 0 or 1;
R₂ is hydrogen, —CH₃ or —CH₂CH₃;
R₃ is hydrogen, —CH₃, or —OCH₃;
R₄ is selected from hydrogen, halogen, —CH₂CH₃, —CHCH₃CH₃, —OCF₃, —C≡N, substituted or unsubstituted aryl, substituted or unsubstituted heteroaryl, cycloalkyl, 5 or 6 membered substituted or unsubstituted saturated or unsaturated heterocycle, —SO₂CH₃, or —CF₃;
R₅ is selected from hydrogen, halogen, —CF₃, substituted or unsubstituted aryl, or
substituted or unsubstituted heteroaryl, or cycloalkyl;
or a pharmaceutically acceptable salt thereof.

In one aspect of the invention, there is provided a compound of Formula III

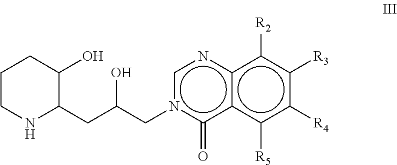

where:
R₂ is hydrogen, —CH₃ or —CH₂CH₃;
R₃ is hydrogen, —CH₃, or —OCH₃;
R₄ is selected from hydrogen, halogen, —CH₂CH₃, —CHCH₃CH₃, —OCF₃, —C≡N, substituted or unsubstituted aryl, substituted or unsubstituted heteroaryl, cycloalkyl, 5 or 6 membered substituted or unsubstituted saturated or unsaturated heterocycle, —SO₂CH₃, or —CF₃;
R₅ is selected from hydrogen, halogen, —CF₃, substituted or unsubstituted aryl, or substituted or unsubstituted heteroaryl, or cycloalkyl;
or a pharmaceutically acceptable salt thereof.

In one aspect of the invention, there is provided a compound of Formula IV

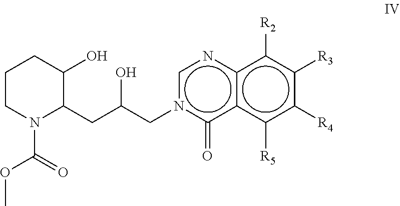

where:
R₂ is hydrogen, —CH₃ or —CH₂CH₃;
R₃ is hydrogen, —CH₃, or —OCH₃;
R₄ is selected from hydrogen, halogen, —CH₂CH₃, —CHCH₃CH₃, —OCF₃, —C≡N, substituted or unsubstituted aryl, substituted or unsubstituted heteroaryl, cycloalkyl, 5 or 6 membered substituted or unsubstituted saturated or unsaturated heterocycle, —SO₂CH₃, or —CF₃;
R₅ is selected from hydrogen, halogen, —CF₃, substituted or unsubstituted aryl, or substituted or unsubstituted heteroaryl, or cycloalkyl;
or a pharmaceutically acceptable salt thereof.

In one aspect of the invention, R₂ is —CH₃, R₃ is hydrogen; R₄ is halogen; and R₅ is hydrogen.

In one aspect of the invention, there is provided a compound of Formula V

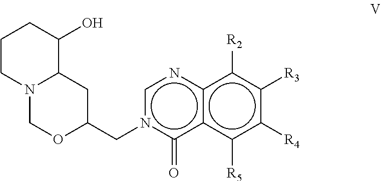

where:
R₂ is hydrogen, —CH₃ or —CH₂CH₃;
R₃ is hydrogen, —CH₃, or —OCH₃;
R₄ is selected from hydrogen, halogen, —CH₂CH₃, —CHCH₃CH₃, —OCF₃, —C≡N, substituted or unsubstituted aryl, substituted or unsubstituted heteroaryl, cycloalkyl, 5 or 6 membered substituted or unsubstituted saturated or unsaturated heterocycle, —SO₂CH₃, or —CF₃;
R₅ is selected from hydrogen, halogen, —CF₃, substituted or unsubstituted aryl, or substituted or unsubstituted heteroaryl, or cycloalkyl;
or a pharmaceutically acceptable salt thereof.

In one aspect of the invention, R₂ is —CH₃, R₃ is hydrogen; R₄ is halogen; and R₅ is hydrogen.

In one aspect of the invention, there is provided a compound selected from:
6-Chloro-3-{3-[(2R,3S)-3-hydroxypiperidin-2-yl]-2-oxopropyl}-8-methylquinazolin-4(3H)-one;
7-Chloro-3-{3-[(2R,3S)-3-hydroxypiperidin-2-yl]-2-oxopropyl}-8-methylquinazolin-4(3H)-one;
6-Bromo-3-{3-[(2R,3S)-3-hydroxypiperidin-2-yl]-2-oxopropyl}-8-methylquinazolin-4(3H)-one;
3-{3-[(2R,3S)-3-Hydroxypiperidin-2-yl]-2-oxopropyl}-7-methoxy-8-methylquinazolin-4(3H)-one;
5-Fluoro-3-{3-[(2R,3S)-3-hydroxypiperidin-2-yl]-2-oxopropyl}quinazolin-4(3H)-one;
6-Ethyl-3-(3-((2R,3S)-3-hydroxypiperidin-2-yl)-2-oxopropyl)-8-methylquinazolin-4(3H)-one;
3-{3-[(2R,3S)-3-Hydroxypiperidin-2-yl]-2-oxopropyl}-8-methyl-6-(trifluoromethoxy)quinazolin-4(3H)-one;
3-{3-[(2R,3S)-3-Hydroxypiperidin-2-yl]-2-oxopropyl}-8-methyl-6-(methylsulfonyl)quinazolin-4(3H)-one;
3-{3-[(2R,3S)-3-Hydroxypiperidin-2-yl]-2-oxopropyl}-8-methyl-6-(trifluoromethyl)quinazolin-4(3H)-one;
3-{3-[(2R,3S)-3-Hydroxypiperidin-2-yl]-2-oxopropyl}-6-isopropyl-8-methylquinazolin-4(3H)-one;
6-Cyclopropyl-3-{3-[(2R,3S)-3-hydroxypiperidin-2-yl]-2-oxopropyl}-8-methylquinazolin-4(3H)-one;
3-{3-[(2R,3S)-3-Hydroxypiperidin-2-yl]-2-oxopropyl}-8-methyl-4-oxo-3,4-dihydroquinazoline-6-carbonitrile;
6-Bromo-8-ethyl-3-{3-[(2R,3S)-3-hydroxypiperidin-2-yl]-2-oxopropyl}quinazolin-4(3H)-one;
8-Ethyl-6-fluoro-3-(3-((2R,3S)-3-hydroxypiperidin-2-yl)-2-oxopropyl)quinazolin-4(3H)-one;
5-(3-Fluorophenyl)-3-{3-[(2R,3S)-3-hydroxypiperidin-2-yl]-2-oxopropyl}quinazolin-4(3H)-one;
6-(3-Fluorophenyl)-3-{3-[(2R,3S)-3-hydroxypiperidin-2-yl]-2-oxopropyl}quinazolin-4(3H)-one;
6-(3-Chlorophenyl)-3-{3-[(2R,3S)-3-hydroxypiperidin-2-yl]-2-oxopropyl}quinazolin-4(3H)-one;
3-{3-[(2R,3S)-3-Hydroxypiperidin-2-yl]-2-oxopropyl}-6-[3-(trifluoromethyl)phenyl]quinazolin-4(3H)-one;
6-(3-Fluorophenyl)-3-{3-[(2R,3S)-3-hydroxypiperidin-2-yl]-2-oxopropyl}-8-methylquinazolin-4(3H)-one;
3-{3-[(2R,3S)-3-Hydroxypiperidin-2-yl]-2-oxopropyl}-8-methyl-6-[3-(trifluoromethyl)phenyl]quinazolin-4(3H)-one;
3-{3-[(2R,3S)-3-Hydroxypiperidin-2-yl]-2-oxopropyl}-8-methyl-6-(pyridin-3-yl)quinazolin-4(3H)-one;
6-(3,4-Difluorophenyl)-3-{3-[(2R,3S)-3-hydroxypiperidin-2-yl]-2-oxopropyl}-8-methylquinazolin-4(3H)-one;
3-{3-[(2R,3S)-3-Hydroxypiperidin-2-yl]-2-oxopropyl}-8-methyl-6-(tetrahydro-2H-pyran-4-yl)quinazolin-4(3H)-one;
3-{3-[(2R,3S)-3-Hydroxypiperidin-2-yl]-2-oxopropyl}-8-methyl-6-(pyrrolidin-1-yl)quinazolin-4(3H)-one;
3-{3-[(2R,3S)-3-Hydroxypiperidin-2-yl]-2-oxopropyl}-5-(trifluoromethyl)quinazolin-4(3H)-one;
3-{3-[(2R,3S)-3-Hydroxypiperidin-2-yl]-2-oxopropyl}-5-[3-(trifluoromethyl)phenyl]quinazolin-4(3H)-one;
5-(3,4-Difluorophenyl)-3-{3-[(2R,3S)-3-hydroxypiperidin-2-yl]-2-oxopropyl}quinazolin-4(3H)-one;
3-{3-[(2R,3S)-3-Hydroxypiperidin-2-yl]-2-oxopropyl}-5-(pyridin-3-yl)quinazolin-4(3H)-one; and
3-{3-[(2R,3S)-3-Hydroxypiperidin-2-yl]-2-oxopropyl}-8-methyl-5-(trifluoromethyl)quinazolin-4(3H)-one;
   or salt thereof.

In one aspect of the invention, there is provided a compound selected from:
6-Chloro-3-{(S)-2-hydroxy-3-[(2R,3S)-3-hydroxypiperidin-2-yl]propyl}-8-methylquinazolin-4(3H)-one;
6-Chloro-3-{(S)-2-hydroxy-3-[(2R,3S)-3-hydroxypiperidin-2-yl]propyl}-7-methylquinazolin-4(3H)-one;
6-Fluoro-3-{(S)-2-hydroxy-3-[(2R,3S)-3-hydroxypiperidin-2-yl]propyl}-7-methylquinazolin-4(3H)-one;
6-Chloro-3-{(S)-2-hydroxy-3-[(2R,3S)-3-hydroxypiperidin-2-yl]propyl}-7-methoxyquinazolin-4(3H)-one;
6-Fluoro-3-{(S)-2-hydroxy-3-[(2R,3S)-3-hydroxypiperidin-2-yl]propyl}-8-methylquinazolin-4(3H)-one;
6-Chloro-3-{(S)-2-hydroxy-3-[(2R,3S)-3-hydroxypiperidin-2-yl]propyl}-8-(methyl-d3)quinazolin-4(3H)-one;
7-Chloro-3-{(S)-2-hydroxy-3-[(2R,3S)-3-hydroxypiperidin-2-yl]propyl}-8-methylquinazolin-4(3H)-one;
6-Bromo-3-{(S)-2-hydroxy-3-[(2R,3S)-3-hydroxypiperidin-2-yl]propyl}-8-methylquinazolin-4(3H)-one;
6-Chloro-8-ethyl-3-{(S)-2-hydroxy-3-[(2R,3S)-3-hydroxypiperidin-2-yl]propyl}quinazolin-4(3H)-one;
3-{(S)-2-Hydroxy-3-[(2R,3S)-3-hydroxypiperidin-2-yl]propyl}-6,8-dimethylquinazolin-4(3H)-one;
5-Fluoro-3-{(S)-2-hydroxy-3-[(2R,3S)-3-hydroxypiperidin-2-yl]propyl}quinazolin-4(3H)-one;
6-Ethyl-3-{(S)-2-hydroxy-3-[(2R,3S)-3-hydroxypiperidin-2-yl]propyl}-8-methylquinazolin-4(3H)-one;
3-{(S)-2-Hydroxy-3-[(2R,3S)-3-hydroxypiperidin-2-yl]propyl}-8-methyl-6-(trifluoromethoxy)quinazolin-4(3H)-one;
3-{(S)-2-Hydroxy-3-[(2R,3S)-3-hydroxypiperidin-2-yl]propyl}-8-methyl-6-(methylsulfonyl)quinazolin-4(3H)-one;
3-{(S)-2-Hydroxy-3-[(2R,3S)-3-hydroxypiperidin-2-yl]propyl}-8-methyl-6-(trifluoromethyl)quinazolin-4(3H)-one;
3-{(S)-2-Hydroxy-3-[(2R,3S)-3-hydroxypiperidin-2-yl]propyl}-6-isopropyl-8-methylquinazolin-4(3H)-one;
6-Cyclopropyl-3-{(S)-2-hydroxy-3-[(2R,3S)-3-hydroxypiperidin-2-yl]propyl}-8-methylquinazolin-4(3H)-one;
3-{(S)-2-Hydroxy-3-[(2R,3S)-3-hydroxypiperidin-2-yl]propyl}-8-methyl-4-oxo-3,4-dihydroquinazoline-6-carbonitrile;
6-Bromo-8-ethyl-3-{(S)-2-hydroxy-3-[(2R,3S)-3-hydroxypiperidin-2-yl]propyl}quinazolin-4(3H)-one;
5-(3-Fluorophenyl)-3-{(S)-2-hydroxy-3-[(2R,3S)-3-hydroxypiperidin-2-yl]propyl}quinazolin-4(3H)-one;
6-(3-Fluorophenyl)-3-{(S)-2-hydroxy-3-[(2R,3S)-3-hydroxypiperidin-2-yl]propyl}quinazolin-4(3H)-one;
6-(3-Chlorophenyl)-3-{(S)-2-hydroxy-3-[(2R,3S)-3-hydroxypiperidin-2-yl]propyl}quinazolin-4(3H)-one;
3-{(S)-2-hydroxy-3-[(2R,3S)-3-hydroxypiperidin-2-yl]propyl}-6-(3-(trifluoromethyl)phenyl)quinazolin-4(3H)-one;
6-(3-Fluorophenyl)-3-{(S)-2-hydroxy-3-[(2R,3S)-3-hydroxypiperidin-2-yl]propyl}-8-methylquinazolin-4(3H)-one;
3-{(S)-2-Hydroxy-3-[(2R,3S)-3-hydroxypiperidin-2-yl]propyl}-8-methyl-6-(3-(trifluoromethyl)phenyl)quinazolin-4(3H)-one;
3-{(S)-2-Hydroxy-3-[(2R,3S)-3-hydroxypiperidin-2-yl]propyl}-8-methyl-6-(pyridin-3-yl)quinazolin-4(3H)-one;
6-(3,4-Difluorophenyl)-3-{(S)-2-hydroxy-3-[(2R,3S)-3-hydroxypiperidin-2-yl]propyl}-8-methylquinazolin-4(3H)-one;
3-{(S)-2-Hydroxy-3-[(2R,3S)-3-hydroxypiperidin-2-yl]propyl}-8-methyl-6-(tetrahydro-2H-pyran-4-yl)quinazolin-4(3H)-one;
3-{(S)-2-Hydroxy-3-[(2R,3S)-3-hydroxypiperidin-2-yl]propyl}-8-methyl-6-(pyrrolidin-1-yl)quinazolin-4(3H)-one;
3-{(S)-2-Hydroxy-3-[(2R,3S)-3-hydroxypiperidin-2-yl]propyl}-5-(trifluoromethyl)quinazolin-4(3H)-one;
3-{(S)-2-Hydroxy-3-[(2R,3S)-3-hydroxypiperidin-2-yl]propyl}-5-[3-(trifluoromethyl)phenyl]quinazolin-4(3H)-one;
5-(3,4-Difluorophenyl)-3-{(S)-2-hydroxy-3-[(2R,3S)-3-hydroxypiperidin-2-yl]propyl}quinazolin-4(3H)-one;
3-{(S)-2-Hydroxy-3-[(2R,3S)-3-hydroxypiperidin-2-yl]propyl}-5-(pyridin-3-yl)quinazolin-4(3H)-one;
(2R,3S)-2-{(S)-3-[6-chloro-8-methyl-4-oxoquinazolin-3(4H)-yl]-2-hydroxypropyl}piperidin-3-ylpivalate;
3-{(S)-2-hydroxy-3-[(2R,3S)-3-hydroxypiperidin-2-yl]propyl}-8-methyl-6-(1-methyl-1H-pyrazol-3-yl)quinazolin-4(3H)-one;
Methyl (2R,3S)-2-((S)-3-(6-chloro-8-methyl-4-oxoquinazolin-3(4H)-yl)-2-hydroxypropyl)-3-hydroxypiperidine-1-carboxylate;
3-((S)-2-hydroxy-3-((2R,3S)-3-hydroxypiperidin-2-yl)propyl)-8-methyl-6-(thiazol-4-yl)quinazolin-4(3H)-one;
6-cyclobutyl-3-{(S)-2-hydroxy-3-[(2R,3S)-3-hydroxypiperidin-2-yl]propyl}-8-methylquinazolin-4(3H)-one;
5-Cyclopropyl-3-{(S)-2-hydroxy-3-[(2R,3S)-3-hydroxypiperidin-2-yl]propyl}quinazolin-4(3H)-one;
3-{(S)-2-hydroxy-3-[(2R,3S)-3-hydroxypiperidin-2-yl]propyl}-8-methyl-6-(piperidin-4-yl)quinazolin-4(3H)-one;
(2R,3S)-2-{(S)-3-[6-chloro-8-methyl-4-oxoquinazolin-3(4H)-yl]-2-hydroxypropyl}piperidin-3-ylpivalate;
3-{(S)-2-hydroxy-3-[(2R,3S)-3-hydroxypiperidin-2-yl]propyl}-8-methyl-6-(1-methyl-1H-pyrazol-3-yl)quinazolin-4(3H)-one; and
6-Chloro-3-{[(3S,4aR,5S)-5-hydroxyhexahydro-1H,3H-pyrido[1,2-c][1,3]oxazin-3-yl]methyl}-8-methylquinazolin-4(3H)-one;
   or salt thereof.

In one aspect of the invention, there is provided a composition comprising a compound of claim 1, or a pharmaceutically acceptable salt thereof, and a pharmaceutically acceptable carrier.

In one aspect of the invention, there is provided a method of inhibiting glutamyl-prolyl tRNA synthetase comprising contacting glutamyl-prolyl tRNA synthetase with a compound of claim 1.

In one aspect of the invention, there is provided a method of treating a Th17-mediated condition comprising administering to a subject in need thereof a compound of claim 1.

In one aspect of the invention, there is provided a method of treating a disease selected from autoimmune diseases, dry eye syndrome, fibrosis, scar formation, angiogenesis, ischemic damage, inflammatory diseases, cancers, musculoskeletal diseases, cardiovascular diseases, transplant rejection, multiple sclerosis, systemic sclerosis and neurodegenerative diseases comprising administering to a subject in need thereof a compound of claim 1.

### DETAILED DESCRIPTION OF THE INVENTION

The following detailed description is provided to aid those skilled in the art in practicing the present invention. Those of ordinary skill in the art may make modifications and variations in the aspects described herein without departing from the spirit or scope of the present disclosure. Unless otherwise defined, all technical and scientific terms used herein have the same meaning as commonly understood by one of ordinary skill in the art to which this disclosure belongs. The terminology used in the description is for describing particular aspects only and is not intended to be limiting.

As used in this application, except as otherwise expressly provided herein, each of the following terms shall have the meaning set forth below. Additional definitions are set forth throughout the application. In instances where a term is not specifically defined herein, that term is given an art-recognized meaning by those of ordinary skill applying that term in context to its use in describing the present invention.

The articles “a” and “an” refer to one or to more than one (i.e., to at least one) of the grammatical object of the article unless the context clearly indicates otherwise. By way of example, “an element” means one element or more than one element.

The term “about” refers to a value or composition that is within an acceptable error range for the particular value or composition as determined by one of ordinary skill in the art, which will depend in part on how the value or composition is measured or determined, i.e., the limitations of the measurement system. For example, “about” can mean within 1 or more than 1 standard deviation per the practice in the art. Alternatively, “about” can mean a range of up to 10% or 20% (i.e., 10% or 20%). For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” should be assumed to be within an acceptable error range for that particular value or composition.

The term “administering” refers to the physical introduction of a composition comprising a therapeutic agent to a subject, using any of the various methods and delivery systems known to those skilled in the art. For example, routes of administration for the compounds of the invention can include enteral, intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some aspects, the compounds of the invention are administered orally. Typical routes of administration can include bucal, intranasal, ophthalmic, oral, osmotic, parenteral, rectal, sublingual, topical, transdermal, or vaginal. Administering can also be performed, for example, once, a plurality of times, and/or over one or more extended periods and can be a therapeutically effective dose or a subtherapeutic dose. Administering may also be performed over an extended period of time such as, for example, by using controlled release, delayed release or slow release formulations.

The term “effective amount” refers to that amount which is sufficient to effect an intended result. The effective amount will vary depending on the subject and disease state being treated, the severity of the affliction and the manner of administration, and may be determined routinely by one of ordinary skill in the art.

The terms “in combination with” and “in conjunction with” refer to administration of one treatment modality in addition to another treatment modality. As such, “in combination with” or “in conjunction with” refers to administration of one treatment modality before, during, or after administration of the other treatment modality to the subject.

The term “pharmaceutically acceptable carrier” includes any and all adjuvants, solvents, dispersion media, coatings, antibacterial and antifungal agents, isotonic and absorption delaying agents, and the like that are physiologically compatible, examples of which are hereinafter described.

The term “salt” refers to a salt form of one or more of the compounds or produges described herein. The term “pharmaceutically acceptable salt” is a type of salt form which are presented to increase the solubility of the compound in the gastric or gastroenteric juices of the patient's gastrointestinal tract in order to promote dissolution and the bioavailability of the compounds. Pharmaceutically acceptable salts include those derived from pharmaceutically acceptable inorganic or organic bases and acids, where applicable. Suitable salts include those derived from alkali metals such as potassium and sodium, alkaline earth metals such as calcium, magnesium and ammonium salts, among numerous other acids and bases well known in the pharmaceutical art. These salts can be made according to common organic techniques employing commercially available reagents. Some anionic salt forms include acetate, acistrate, besylate, bromide, chloride, citrate, fumarate, glucouronate, hydrobromide, hydrochloride, hydroiodide, iodide, lactate, maleate, mesylate, nitrate, pamoate, phosphate, succinate, sulfate, tartrate, tosylate, and xinofoate. Some cationic salt forms include ammonium, aluminum, benzathine, bismuth, calcium, choline, diethylamine, diethanolamine, lithium, magnesium, meglumine, 4-phenylcyclohexylamine, piperazine, potassium, sodium, tromethamine, and zinc. The compounds may be present as a neutral compound, salt form, or pharmaceutically acceptable salt form thereof.

The term “prodrug” refers to a precursor of a drug which may be administered in an altered or less active form. The prodrug may be converted into the active drug form in physiological environments by hydrolysis or other metabolic pathways. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press.

The terms “subject” and “patient” refer any human or nonhuman animal. The term “nonhuman animal” includes, but is not limited to, vertebrates such as nonhuman primates, sheep, dogs, and rodents such as mice, rats and guinea pigs. In some aspects, the subject is a human. The terms, “subject” and “patient” are used interchangeably herein.

The term, “subtherapeutic dose” refers a dose of a therapeutic agent (e.g., a compound of the invention) that is lower than the usual or typical dose of the therapeutic agent when administered alone for the treatment of a disease (e.g., fibrosis).

The terms “therapeutically effective amount”, “therapeutically effective dosage” and “therapeutically effective dose” of an agent (also sometimes referred to herein as a “drug”) refers to any amount of the agent that, when used alone or in combination with another agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of an agent to promote disease regression can be evaluated using a variety of methods known to the skilled practitioner, such as in human subjects during clinical trials, in animal model systems predictive of efficacy in humans, or by assaying the activity of the agent in in vitro assays. In certain aspects, the therapeutically effective amount prevents the development or recurrence of the disease entirely. “Inhibiting” the development or recurrence of a disease means either lessening the likelihood of the disease's development or recurrence, or preventing the development or recurrence of the disease entirely.

The term “treatment” refers to any treatment of a condition or disease in a subject and may include: (i) preventing the disease or condition from occurring in the subject which may be predisposed to the disease but has not yet been diagnosed as having it; (ii) inhibiting the disease or condition, i.e., arresting its development; relieving the disease or condition, i.e., causing regression of the condition; or (iii) ameliorating or relieving the conditions caused by the disease, i.e., symptoms of the disease. Treatment could be used in combination with other standard therapies or alone. Treatment or “therapy” of a subject also includes any type of intervention or process performed on, or the administration of an agent to, the subject with the objective of reversing, alleviating, ameliorating, inhibiting, slowing down or preventing the onset, progression, development, severity or recurrence of a symptom, complication or condition, or biochemical indicia associated with a disease.
The scope of any instance of a variable, including R₁, R₂, R₃, R₄, R₅, E and Z, can be used independently with the scope of any other instance of a variable substituent. As such, the invention includes combinations of the different aspects.

Some compounds of the invention may exist in stereoisomeric forms. The invention includes all stereoisomeric and tautomeric forms of the compounds.

The invention is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. Such compounds may have a variety of potential uses, for example as standards and reagents in determining biological activity. In the case of stable isotopes, such compounds may have the potential to favorably modify biological, pharmacological, or pharmacokinetic properties.
In some variations, the compounds disclosed herein may be varied such that from 1 to “n” hydrogens attached to a carbon atom is/are replaced by deuterium, in which “n” is the number of hydrogens in the molecule. Such compounds may exhibit increased resistance to metabolism and are thus useful for increasing the half life of the compound when administered to a subject. See, for example, Foster, “Deuterium Isotope Effects in Studies of Drug Metabolism”, Trends Pharmacol. Sci. 5(12):524-527 (1984). Such compounds are synthesized by means well known in the art, for example by employing starting materials in which one or more hydrogens have been replaced by deuterium.

Deuterium labeled or substituted therapeutic compounds of the disclosure may have improved drug metabolism and pharmacokinetics (DMPK) properties, relating to absorption, distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. It is understood that deuterium in this context is regarded as a substituent in the compounds provided herein.

Preferably, the compounds of the invention do not promote the formation of glutathione (“GSH”) adducts in any significant amounts. In the liver, GSH can form stable addusts with many reactive species, e.g., active drug metabolites. In accordance with the present invention, the compounds surprisingly do not form significant amounts of GSH adducts. As a result, the GSH pool in the liver of patients is substantially not reduced by administration of the compounds of the invention.

### Synthetic Methods

The compounds if the invention may be made by methods known in the art including those described below and including variations within the skill of the art. Some reagents and intermediates are known in the art. Other reagents and intermediates can be made by methods known in the art using readily available materials. The following methods are for illustrative purposes and are not intended to limit the scope of the invention. It will be appreciated by those skilled in the art that there are a number of methods available for the synthesis of these compounds and that their synthesis is not limited to the methods provided in the following examples. Variations of the compounds and the procedures to make them which are not illustrated are within the skill of the art. The variables describing general structural formulas and features in the synthetic schemes are distinct from and should not be confused with the variables in the claims or the rest of the description. These variables are meant only to illustrate how to make some of the compounds of the invention.

Common abbreviations are used herein, are may include terms such as, for example: “1×” for once, “2×” for twice, “3×” for thrice, “° C.” for degrees Celsius, “aq” for aqueous, “Col” for column, “eq” for equivalent or equivalents, “g” for gram or grams, “mg” for milligram or milligrams, “L” for liter or liters, “mL” for milliliter or milliliters, “μL” for microliter or microliters, “N” for normal, “M” for molar, “nM” for nanomolar, “mol” for mole or moles, “mmol” for millimole or millimoles, “min” for minute or minutes, “h” for hour or hours, “rt” for room temperature, “RT” for retention time, “ON” for overnight, “atm” for atmosphere, “psi” for pounds per square inch, “conc.” for concentrate, “aq” for “aqueous”, “sat” or “sat'd” for saturated, “MW” for molecular weight, “mw” or “wave” for microwave, “mp” for melting point, “Wt” for weight, “MS” or “Mass Spec” for mass spectrometry, “ESI” for electrospray ionization mass spectroscopy, “HR” for high resolution, “HRMS” for high resolution mass spectrometry, “LCMS” for liquid chromatography mass spectrometry, “HPLC” for high pressure liquid chromatography, “RP HPLC” for reverse phase HPLC, “TLC” or “tlc” for thin layer chromatography, “NMR” for nuclear magnetic resonance spectroscopy, “nOe” for nuclear Overhauser effect spectroscopy, “¹H” for proton, “δ” for delta, “s” for singlet, “d” for doublet, “t” for triplet, “q” for quartet, “m” for multiplet, “br” for broad, “Hz” for hertz, and “α”, “β”, “R”, “S”, “E”, and “Z” are stereochemical designations familiar to one skilled in the art. Others are listed in Table 1 below.

TABLE 1

| | |
|---|---|
| Ac | Acetic |
| AcOH | acetic acid |
| ACN (or MeCN) | Acetonitrile |
| APF | aminophenyl fluorescein |
| BINAP | 2,2′-bis(diphenylphosphino)-1,1′-binaphthyl |
| BISPIN | Bis(pinacolato)diboron |
| Bn | Benzyl |
| Boc | tert-butyl carbonyl |
| Boc₂O | Di-tert-butyl dicarbonate |
| Bu | Butyl |
| dba (Pd₂(dba)₃) | dibenzylideneacetone |
| CMBP | cyanomethylenetributylphosphorane |
| DCM | dichloromethane |
| DEAD | diethyl azodicarboxylate |
| DIAD | diisopropyl azodicarboxylate |
| Diamide | N,N,N′,N′-Tetramethylazodicarbonamide |
| | (1,1′-Azobis(N,N-dimethylformamide)) |
| DIEA | diisopropylethylamine |
| DMAP | 4-dimethylaminopyridine |
| DME | Dimethoxyethane |
| DMF | dimethylformamide |
| DMSO | dimethyl sulfoxide |
| dppf | 1,1′-bis(diphenylphosphino)ferrocene |
| (DtBPF)PdCl₂ | 1.1′-bis(di-tert-butylphosphino)ferrocene palladium |
| | dichloride |
| Et | ethyl |
| EtOH | Ethanol |
| EtOAc | ethyl acetate |
| HATU | 2-(7-Aza-1H-benzotriazole-1-y1)-1,1,3,3- |
| | tetramethyluronium hexafluorophosphate |
| HBTU | 2-(1H-Benzotriazole-1-y1)-1,1,3,3- |
| | tetramethyluronium hexafluorophosphate |
| i-Bu | isobutyl |
| i-Pr | isopropyl |
| LAH | lithium aluminum hydride |
| Me | Methyl |
| MeOH | methanol |
| NMM | N-methylmorpholine |
| NMP | N-Methylpyrrolidone |
| PCC | pyridinium chlorochromate |
| Ph | Phenyl |
| Pr | Propyl |
| RT or rt | Room temperature |
| t-Bu | tert-butyl |
| TBDMS-Cl | t-butyldimethylchlorosilane |
| TBDMS | t-butyldimethylsilyl |
| TBDPS | t-butyldiphenylsilyl |
| TBDPS-Cl | t-butyldiphenylchlorosilane |
| TEA | Triethylamine |
| TFA | Trifluoroacetic acid |
| THF | tetrahydrofuran |
| TMAD | N,N,N′,N′-Tetramethylazodicarbonamide |
| | (1,1′-Azobis(N,N′-dimethylformamide)) |
| Ts | Tosyl |

It will also be recognized that another major consideration in the planning of any synthetic route in this field is the judicious choice of the protecting group used for protection of the reactive functional groups present in the compounds described in this invention. An authoritative account describing the many alternatives to the trained practitioner is Greene, T. W. et al., Protecting Groups in Organic Synthesis, 4th Edition, Wiley (2007)).

Compounds having the general Formula (Q): wherein A and B defined below as Ar¹, and Ar² respectively, can be prepared by the following one or more of the synthetic Schemes.

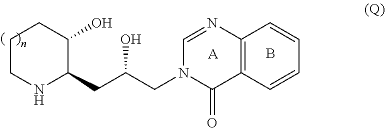

Halofuginone and Halofuginol derivatives of this invention wherein rings A and B are substituted phenyl and or heteroaryl rings can be prepared by the general route shown in Scheme 1, starting from a benzyl (2R,3S)-3-(((benzyloxy)carbonyl)oxy)-2-(2-oxopropyl)piperidine-1-carboxylate, which was synthesized using methods known to one skilled in the art for these types of couplings (see for example Satoshi et al., Synthesis. 2008, 19, 3081). The methyl group in 1 was brominated using a two-step, one-pot procedure involving the formation of a trimethylsilyl enol ether. The crude bromo compound was then treated with quinazoline derivatives (2) in the presence of base to form Cbz protected halofuginone derivatives 3. The protection in 3 was removed either with 6M aq. HCl or trifluoroacetic acid and the crude compound obtained was directly converted to corresponding tert-butyl carbamate 3. The Boc deprotection of 4 using acids (HCl, TFA) afforded the halofuginone derivatives 5 as the corresponding acid salt. The reduction of Boc-protected halofuginone derivatives 4 with NaBH₄ provided diastereomers which were separated by RP HPLC. The desired isomer 6 was deprotected to the corresponding halofuginol derivatives (e.g. 7) as acid salt.

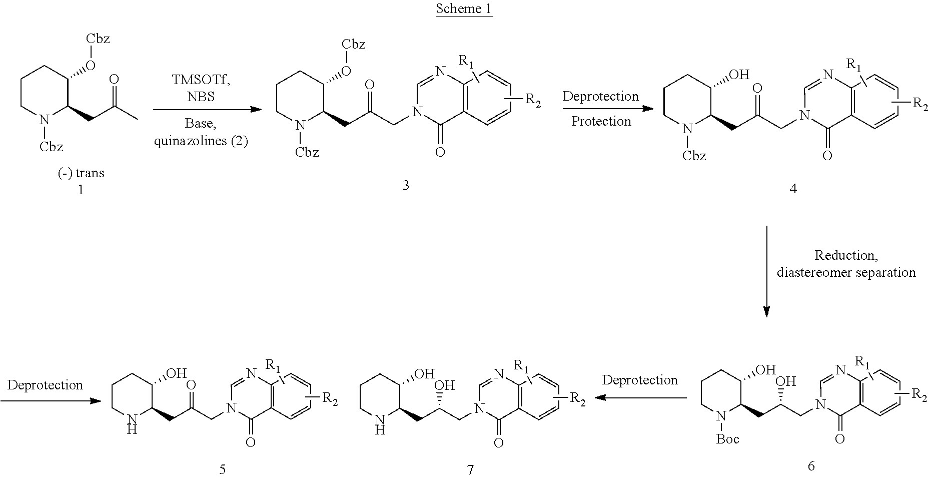

### Pharmaceutical Compositions

Liquid dosage forms for oral and parenteral administration include, but are not limited to, pharmaceutically acceptable sprays, e.g., for intranasal administration, emulsions, microemulsions, solutions, suspensions, syrups, and elixirs. In addition to the active agents, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, com, germ, olive, castor, and sesame oils), glycerol, tetrahydrofurfuryl alcohol, polyethylene glycols and fatty acid esters of sorbitan, and mixtures thereof. Besides inert diluents, oral compositions can also include adjuvants such as wetting agents, emulsifying and suspending agents, sweetening, flavoring, and perfuming agents. In certain aspects for parenteral administration, agents of the invention are mixed with solubilizing agents such CREMOPHOR EL® (polyethoxylated castor oil), alcohols, oils, modified oils, glycols, polysorbates, cyclodextrins, polymers, and combinations thereof.

Injectable preparations, for example, sterile injectable aqueous or oleaginous suspensions may be formulated according to the known art using suitable dispersing or wetting agents and suspending agents. Sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in tum, may depend upon crystal size and crystalline form. Alternatively, delayed absorption of a parenterally administered drug form can be accomplished by dissolving or suspending the drug in an oil vehicle. Injectable depot forms are made by forming microencapsule matrices of the drug in biodegradable polymers such as poly(lactide-co-glycolide). Depending upon the ratio of drug compound to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active agent.

Solid dosage forms for oral administration include capsules, tablets, pills, films, powders, and granules. In such solid dosage forms, the active agent is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polethylene glycols and the like.

Formulations suitable for topical administration include liquid or semi-liquid preparations such as liniments, lotions, gels, applicants, oil-in-water or water-in-oil emulsions such as creams, ointments, or pastes; or solutions or suspensions such as drops. Formulations for topical administration to the skin surf ace can be prepared by dispersing the drug with a dermatologically acceptable carrier such as a lotion, cream, ointment, or soap. Useful carriers are capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used. Ophthalmic formulation, ear drops, and eye drops are also contemplated as being within the scope of this invention. Additionally, the present invention contemplates the use of transdermal patches, which have the added advantage of providing controlled delivery of an agent to the body. Such dosage forms can be made by dissolving or dispensing the agent in the proper medium. Absorption enhancers can also be used to increase the flux of the agent across the skin. The rate can be controlled by either providing a rate controlling membrane or by dispersing the agent in a polymer matrix or gel.

Additionally, the carrier for a topical formulation can be in the form of a hydroalcoholic system (e.g., quids and gels), an anhydrous oil or silicone based system, or an emulsion system, including, but not limited to, oil-in-water, water-in-oil, water-in-oil-in-water, and oil-in-water-in-silicone emulsions. The emulsions can cover a broad range of consistencies including thin lotions (which can also be suitable for spray or aerosol delivery), creamy lotions, light creams, heavy creams, and the like. The emulsions can also include microemulsion systems. Other suitable topical carriers include anhydrous solids and semisolids (such as gels and sticks); and aqueous based mousse systems.

It will also be appreciated that the compounds and pharmaceutical compositions of the present invention can be employed in combination therapies, that is, the agents and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another agent), or they may achieve different effects (e.g., control of any adverse effects).

The present invention also provides a pharmaceutical package or kit comprising one or more containers filled with one or more of the ingredients of the pharmaceutical compositions of the present invention, and in certain aspects, includes an additional approved therapeutic agent for use as a combination therapy. Optionally associated with such container(s) can be a notice in the form prescribed by a governmental agency regulating the manufacture, use or sale of pharmaceutical products, e.g., the U.S. Food and Drug Administration, which notice reflects approval by the agency of manufacture, use or sale for human administration.

### Methods of Treatment

Compounds of the present invention may be useful in the treatment of disorders associated with glutamyl-prolyl tRNA synthetase (EPRS) inhibition, Th17 differentiation, and amino acid starvation response (AAR) induction, such as chronic inflammation, fibrosis, autoimmune diseases, scarring, angiogenesis, transplant, implant, or device rejection, ischemic damage, viral infections, and neurodegenerative disorders. The compounds may also be used in treating protozoal infections such as malaria by inhibiting the prolyl tRNA synthetase of the protozoa.

In some aspects of the invention, the compounds inhibit tRNA synthetase. In particular, compounds of the present invention may inhibit glutamyl-prolyl tRNA synthetase (EPRS) (e.g., mammalian EPRS, human EPRS). In certain aspects, compounds of the present invention inhibit non-metazoan prolyl tRNA synthetase (e.g., protozoa!prolyl tRNA synthetase). In certain aspects, the compounds suppress the differentiation of a subset of effector T-cells (i.e., Th17 cells). In certain aspects, the compounds suppress IL-17 production. In certain aspects, the compounds activate the amino acid starvation response (AAR).

The biological activity of provided compounds may make them useful in the treatment of a variety of diseases and conditions. For example, in certain aspects, the compounds are useful in the treatment of diseases and conditions associated with IL-17 production, such as arthritis, inflammatory bowel disease, psoriasis, multiple sclerosis, lupus, asthma, dry eye syndrome, and other autoimmune and/or inflammatory diseases. In certain other aspects, the compounds of the present invention suppress pro-fibrotic gene expression; therefore, they are useful in treating or preventing fibrosis. In some aspects, the compounds of the present invention inhibit viral gene expression, replication, and maturation. In other aspects, the compounds of the present invention protect organs from stress. In certain aspects, the compounds suppress the synthesis of toxic proteins such as polyglutamine-containing proteins that cause neurodegenerative diseases such as Huntington's disease. In some aspects, the compounds promote autophagy. In certain aspects, the compounds inhibit the synthesis of praline-rich proteins such as collagen. In certain other aspects, the compounds inhibit angiogenesis. In certain aspects, the compounds are useful for treating protozoal infections.

Certain compounds of the present invention may be useful in treating inflammatory diseases or autoimmune diseases, such as inflammatory bowel disease, multiple sclerosis, rheumatoid arthritis, lupus, psoriasis, scleroderma, or dry eye syndrome. In certain aspects, provided compounds are useful in the treatment of cardiovascular diseases, diseases involving angiogenesis, neurodegenerative diseases, or protein aggregation disorders. In certain aspects, provided compounds are useful in the treatment of T-cell neoplasms such as mature T-cell leukemias, nodal peripheral T-cell lymphomas (PTCL), extranodal PTCLs, and cutaneous T-cell lymphomas (CTCL). Certain compounds of the present invention are also useful as anti-scarring agents. In some aspects, the compounds are useful in treating viral infections. In other aspects, the compounds are useful in the treatment or prevention of restenosis.

In certain aspects of the invention, the compounds may be used to treat musculoskeletal diseases such as Duchene Muscular Dystrophy, cardiovascular diseases, including cardiomyopathy, fibrotic diseases, including lung, liver and cardiac fibrosis, transplant rejection, multiple sclerosis, scleroderma, systemic sclerosis, skin fibrotic conditions including post-surgery scarring, or keloids.

In certain aspects of the invention, the compounds may be used to treat cancer. The term “cancer” refers to a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. “Cancer” includes primary, metastatic and recurrent cancers as well as a precancerous condition, i.e., a state of disordered morphology of cells that is associated with an increased risk of cancer. The term “cancer” includes, but is not limited to, the following proliferative diseases: Acute Lymphoblastic Leukemia (ALL), Acute Myeloid Leukemia (AML), Adrenocortical Carcinoms, Childhood cancers, AIDS-Related Cancers, Kaposi Sarcoma, AIDS-Related Lymphoma, Primary CNS Lymphoma, Anal Cancer, Astrocytomas, Atypical Teratoid/Rhabdoid Tumor, Basal Cell Carcinoma, Skin Cancer (Nonmelanoma), Bile Duct Cancer, Bladder Cancer, Bone Cancer, Ewing Sarcoma Family of Tumors, Osteosarcoma and Malignant Fibrous Histiocytoma, Brain Stem Glioma, Atypical Teratoid/Rhabdoid Tumor, Embryonal Tumors, Germ Cell Tumors, Craniopharyngioma, Ependymoma, Breast Cancer, Bronchial Tumors, Burkitt Lymphoma, Non-Hodgkin Lymphoma, Carcinoid Tumor, Gastrointestinal Carcinoma, Cardiac (Heart) Tumors, Primary Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colon Cancer, Colorectal Cancer, Craniopharyngioma, Cutaneous T-Cell Lymphoma, Mycosis Fungoides and Sezary Syndrome, Ductal Carcinoma In Situ (DCIS), Embryonal Tumors, Endometrial Cancer, Ependymoma, Esophageal Cancer, Esthesioneuroblastoma, Extracranial Germ Cell Tumor, Extragonadal Germ Cell Tumor, Eye Cancer, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors (GIST), Germ Cell Tumor, Ovarian, Testicular, Gestational Trophoblastic Disease, Glioma, Hairy Cell Leukemia, Head and Neck Cancer, Hepatocellular (Liver) Cancer, Histiocytosis, Langerhans Cell, Hodgkin Lymphoma, Hypopharyngeal Cancer, Islet Cell Tumors, Pancreatic Neuroendocrine Tumors, Kaposi Sarcoma, Kidney, Renal Cell, Langerhans Cell Histiocytosis, Laryngeal Cancer, Leukemia, Acute Lymphoblastic (ALL), Acute Myeloid (AML), Chronic Lymphocytic (CLL), Chronic Myelogenous (CML), Hairy Cell, Lip and Oral Cavity Cancer, Liver Cancer (Primary), Lung Cancer, Non-Small Cell, Small Cell, Lymphoma, Hodgkin, Non-Hodgkin, Macroglobulinemia, Waldenström, Male Breast Cancer, Melanoma, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia, Chronic (CML), Myeloid Leukemia, Acute (AML) Myeloma, Multiple, Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip and Oropharyngeal Cancer, Osteosarcoma and Malignant Fibrous Histiocytoma of Bone, Ovarian Cancer, Low Malignant Potential Tumor, Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Pregnancy and Breast Cancer, Primary CNS Lymphoma, Primary Peritoneal Cancer, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Rhabdomyosarcoma, Uterine, Small Intestine Cancer, Soft Tissue Sarcoma, Sqamous Cell Carcinoma, Squamous Neck Cancer with Occult Primary, Metastatic, Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Unknown Primary, Ureter and Renal Pelvis, Transitional Cell Cancer, Urethral Cancer, Uterine Cancer, Endometrial, Uterine Sarcoma, Vaginal Cancer, Vulvar Cancer, Waldenström Macroglobulinemia, and Wilms Tumor.

In one aspect of the invention, the cancer is selected from melanoma cancer, renal cancer, prostate cancer, breast cancer, colon cancer, lung cancer, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the CNS, primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and any combinations thereof.

The compounds of the invention are generally administered in a therapeutically effective amount or sub-therapeutic amount. Actual dosage levels of the active ingredient or ingredients in the pharmaceutical compositions of the present invention can be varied so as to obtain an amount of the active ingredient which is effective to achieve the desired therapeutic response for a particular patient, composition, and mode of administration, without being unduly toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A composition of the present invention comprising an immunotherapeutic agent can be administered via one or more routes of administration using one or more of a variety of methods well known in the art. As will be appreciated by the skilled artisan, the route and/or mode of administration will vary depending upon the desired results. The compounds of the invention may be administered, for example, at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect. The desired dosage may be delivered three times a day, two times a day, once a day, every other day, every third day, every week, every two weeks, every three weeks, or every four weeks. In certain aspects, the desired dosage may be delivered using multiple administrations (e.g., two, three, four, five, six, seven, eight, nine, ten, eleven, twelve, thirteen, fourteen, or more administrations). In certain aspects, the compounds are administered at a dose that is below the dose at which the agent causes non-specific effects. In certain aspects, the compounds are administered at a dose that does not cause generalized immunosuppression in a subject.

### EXAMPLES

Other features of the invention will become apparent in the course of the following descriptions of exemplary embodiments that are given for illustration of the invention and are not intended to be limiting thereof.

The following methods were used in the exemplified Examples, except where noted otherwise. Purification of intermediates and final products was carried out via either normal or reverse phase chromatography. Normal phase chromatography was carried out using prepacked SiO₂ cartridges eluting with either gradients of hexanes and ethyl acetate or DCM and MeOH unless otherwise indicated. Reverse phase preparative HPLC was carried out using C18 columns with UV 220 nm or prep LCMS detection eluting with gradients of Solvent A (90% water, 10% MeOH, 0.1% TFA) and Solvent B (10% water, 90% MeOH, 0.1% TFA) or with gradients of Solvent A (95% water, 5% ACN, 0.1% TFA) and Solvent B (5% water, 95% ACN, 0.1% TFA) or with gradients of Solvent A (95% water, 2% ACN, 0.1% HCOOH) and Solvent B (98% ACN, 2% water, 0.1% HCOOH) or with gradients of Solvent A (95% water, 5% ACN, 10 mM NH₄OAc) and Solvent B (98% ACN, 2% water, 10 mM NH₄OAc) or with gradients of Solvent A (98% water, 2% ACN, 0.1% NH₄OH) and Solvent B (98% ACN, 2% water, 0.1% NH₄OH).

LC/MS Methods Employed in Characterization of Examples. Reverse phase analytical HPLC/MS was performed on a Waters Acquity system coupled with a Waters MICROMASS® ZQ Mass Spectrometer.
Method A: Linear gradient of 0 to 100% B over 3 min, with 0.75 min hold time at 100% B;
UV visualization at 220 nm
Column: Waters BEH C18 2.1×50 mm
Flow rate: 1.0 mL/min
Solvent A: 0.1% TFA, 95% water, 5% acetonitrile
Solvent B: 0.1% TFA, 5% water, 95% acetonitrile
   Method B: Linear gradient of 0 to 100% B over 3 min, with 0.75 min hold time at 100% B;
UV visualization at 220 nm
Column: Waters BEH C18 2.1×50 mm
Flow rate: 1.0 mL/min
Solvent A: 10 mM ammonium acetate, 95% water, 5% acetonitrile
Solvent B: 10 mM ammonium acetate, 5% water, 95% acetonitrile
   Analytical HPLC: Methods Employed in Characterization of Examples Products were analyzed by reverse phase analytical HPLC: carried out on a Shimadzu Analytical HPLC: system running Discovery VP software. RT=retention time.
   Method A: Column: X-Bridge C8 (50×4.6) mm, 3.5 m, Mobile Phase A: 0.1% TFA in H₂O, Mobile Phase B: 0.1% TFA in ACN; Flow Rate: 2.0 mL/min
   Method B: Column: X-Bridge C8 (50×4.6) mm, 3.5 m. Mobile Phase C: 10 mM NH₄HCO₃ in H₂O, Mobile Phase D: ACN, Flow Rate: 1.0 mL/min
   Method C: Column: Kinetex EVO C18 (4.6×100) mm, 2.6 m, Buffer: 0.05% TFA in water

### Mobile Phase C: Buffer: Acetonitrile (95:5), Mobile Phase D: Acetonitrile: Buffer (95:5)

Method D: Column: X-Bridge Phenyl (150×4.6 mm) 3.5 m, Mobile phase A: 0.05% TFA in Water: Acetonitrile (95:5), Mobile phase B: Acetonitrile: 0.05% TFA in Water (95:5)
Method E: Column: Kinetex biphenyl (4.6×100) mm, 2.6 m Buffer: 0.05% TFA in water

### Mobile Phase C: Buffer: Acetonitrile (95:5) Mobile Phase D: Acetonitrile: Buffer (95:5)

Method F: Column: Sunfire C18 (4.6×150) mm, 3.5 m; Mobile Phase A: 0.05% TFA in water: Acetonitrile: 95:05; Mobile Phase B: Acetonitrile: 0.05% TFA in water: 95:05
Method G: Column: Sunfire C18 (150×4.6 mm) 5 m, Mobile phase A: 10 mM ammonium acetate in Water Mobile phase B: Acetonitrile
Method H: Column: Kinetex EVO C18 (4.6×100) mm, 2.6 m, Mobile Phase C: 10 mM ammonium acetate in water, Mobile Phase D: Acetonitrile
Method I: Column: Xbridge Phenyl (250×4.6 mm) 5 m, Mobile Phase A: 10 mM ammonium bicarbonate-pH-9.5, Mobile Phase B: Acetonitrile
NMR Employed in Characterization of Examples. ¹H NMR spectra were obtained with Bruker or JEOL® Fourier transform spectrometers operating at frequencies as follows:
¹H NMR: 400 MHz (Bruker or JEOL®) or 500 MHz (Bruker or JEOL®). ¹³C NMR: 100 MHz (Bruker or JEOL®). Spectra data are reported in the format: chemical shift (multiplicity, coupling constants, number of hydrogens). Chemical shifts are specified in ppm downfield of a tetramethylsilane internal standard (δ units, tetramethylsilane=0 ppm) and/or referenced to solvent peaks, which in ¹H NMR spectra appear at 2.49 ppm for CD₂HSOCD₃, 3.30 ppm for CD₂HOD, 1.94 for CD₃CN, and 7.24 ppm for CHCl₃, and which in ¹³C NMR spectra appear at 39.7 ppm for CD₃SOCD₃, 49.0 ppm for CD₃OD, and 77.0 ppm for CDCl₃. All ¹³C NMR spectra were proton decoupled.

### Intermediate 2: 6-Chloro-8-methylquinazolin-4(3H)-one

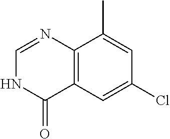

A mixture of 2-amino-5-chloro-3-methyl-benzoic acid (15 g, 81.08 mmol) and formamide (150 mL) was heated at 150° C. for 12 h. Upon completion of the reaction, the reaction mixture was cooled to room temperature and quenched with ice (˜200 g). The resulting precipitate was collected by filtration, washed with water, and dried to get 6-chloro-8-methyl-3H-quinazolin-4-one (7 g; 45%) as an off-white solid. LCMS (ESI) m/z: 195.3 [M+H]⁺; ¹H NMR (300 MHz, DMSO-d₆) δ 12.44 (br s, 1H), 8.16 (s, 1H), 7.90 (d, J=3.2 Hz, 1H), 7.76 (d, J=3.2 Hz, 1H), 2.52 (s, 3H).

### Intermediate 3: Benzyl (2R,3S)-3-{[(benzyloxy)carbonyl]oxy}-2-{3-[6-chloro-8-methyl-4-oxoquinazolin-3(4H)-yl]-2-oxopropyl}piperidine-1-carboxylate

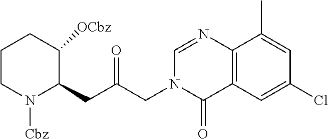

To a solution of benzyl (2R,3S)-2-acetonyl-3-benzyloxycarbonyloxy-piperidine-1-carboxylate (Sukemoto S., Synthesis 2008, 3081-7) (2 g, 4.70 mmol) in CH₂Cl₂ (20 mL) were added DIPEA (1.6 mL, 9.41 mmol) and trimethylsilyl trifluoromethanesulfonate (2.09 g, 1.7 mL, 9.41 mmol) at rt. The mixture was stirred at rt for 30 min then N-bromosuccinimide (1.26 g, 7.05 mmol) in CH₂Cl₂ (20 mL) was added. The reaction mixture was stirred at rt for 1 h then quenched with sat. Na₂S203 (50 mL), extracted with CH₂Cl₂ (50×2 mL). The combined organic layers were washed with water (50 mL), brine (50 mL), dried over Na₂SO₄ and concentrated in vacuo.

The solution of the above residue in THE (20 mL) were treated with 6-chloro-8-methyl-3H-quinazolin-4-one (555 mg, 2.86 mmol), K₂CO₃ (741 mg, 5.36 mmol) and stirred at rt for 16 h. The reaction mixture was filtered and concentrated. The residue was subjected to flash column chromatography (PE/EA, 4:6) to afford the title compound (1.04 g, 36%) as a brown liquid.

LCMS (ESI) m/z: 618.0 [M+H]⁺; ¹H NMR (400 MHz, DMSO-d₆) δ 8.22 (s, 1H), 7.91 (d, J=2.4 Hz, 1H), 7.81 (d, J=2.4 Hz, 1H), 7.40-7.28 (m, 10H), 5.20-4.87 (m, 6H), 4.80-4.60 (m, 1H), 3.99-3.90 (m, 1H), 3.20-2.80 (m, 4H), 2.67 (s, 3H), 1.99-1.90 (m, 1H), 1.88-1.72 (m, 1H), 1.60-1.51 (m, 1H), 1.50-1.38 (m, 1H).

### Intermediate 4: Tert-butyl (2R,3S)-2-{3-[6-chloro-8-methyl-4-oxoquinazolin-3(4H)-yl]-2-oxopropyl}-3-hydroxypiperidine-1-carboxylate

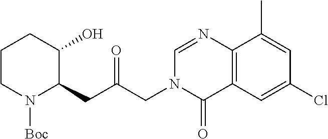 [SMILES: Cc1cc(Cl)cc2c(=O)n(CC(=O)C[C@@H]3[C@@H](O)CCCN3C(=O)OC(C)(C)C)cnc12]

The reaction mixture of benzyl (2R,3S)-3-{[(benzyloxy)carbonyl]oxy}-2-{3-[6-chloro-8-methyl-4-oxoquinazolin-3(4H)-yl]-2-oxopropyl}piperidine-1-carboxylate (1.04 g, 1.68 mmol) in TFA (10 mL) was heated at 50° C. for 12 h. The reaction mixture was cooled to rt, concentrated to get the crude product as brown liquid.
To a solution of above crude in CH₂Cl₂ (50 mL) were added triethylamine (310 mg, 3.2 mmol) and di-tert-butyl dicarbonate (360 mg, 1.8 mmol) at 0° C. The reaction mixture was stirred at rt for 8 h. After stirring for 8 h, the mixture was concentrated and the residue was purified by column chromatography on silica gel (PE/EtOAc, 2:8) to give title compound (470 mg, 62%) as an off-white solid.
   LCMS (ESI) m/z: 450.2 [M+H]⁺; ¹H NMR (400 MHz, CD₃OD) δ 8.15 (s, 1H), 8.02 (dd, J=2.8, 0.4 Hz, 1H), 7.70 (dd, J=2.8, 0.8 Hz, 1H), 5.06 (s, 2H), 4.70 (t, J=6.4 Hz, 1H), 4.02-3.92 (m, 1H), 3.84-3.79 (m, 1H), 3.01-2.91 (m, 2H), 2.86-2.78 (m, 1H), 2.61 (s, 3H), 1.93-1.78 (m, 2H), 1.77-1.69 (m, 1H), 1.47 (s, 9H), 1.43-1.38 (m, 1H).

### Example 1: 6-Chloro-3-{3-[(2R,3S)-3-hydroxypiperidin-2-yl]-2-oxopropyl}-8-methylquinazolin-4(3H)-one hydrochloride

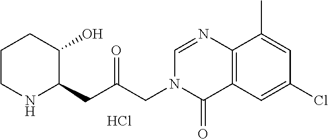 [SMILES: Cc1cc(Cl)cc2c(=O)n(CC(=O)C[C@H]3NCCC[C@@H]3O)cnc12 ; Cl]

To a solution of tert-butyl (2R,3S)-2-{3-[6-chloro-8-methyl-4-oxoquinazolin-3(4H)-yl]-2-oxopropyl}-3-hydroxypiperidine-1-carboxylate (20 mg, 0.044 mmol) in CH₂Cl₂ (5 mL) was added 4M HCl in dioxane (0.5 mL) at 0° C. under nitrogen atmosphere. The reaction mixture was stirred at rt for 1 h. The reaction mixture was concentrated to get the crude product. The crude product triturated with diethyl ether (5 mL) to get title compound (10 mg, 58%) as a white solid.

LCMS (ESI) m/z: 350 [M+H]⁺; ¹H NMR (400 MHz, DMSO-d₆): δ 8.83 (br s, 2H), 8.29 (s, 1H), 7.93 (d, J=1.8 Hz 1H), 7.83 (s, 1H), 5.60 (d, J=5.4 Hz, 1H), 5.09 (s, 2H), 3.52-3.50 (m, 1H), 3.30-3.14 (m, 3H), 2.94-2.88 (m, 2H), 2.56 (s, 3H), 1.94-1.92 (m, 1H), 1.83-1.79 (m, 1H), 1.65-1.62 (m, 1H), 1.47-1.44 (m, 1H). Analytical HPLC RT=2.76 min (Method A)

The following Examples in Table 2 were prepared by using the same procedure as described in Example 1 by de-protection of the appropriate Boc Intermediate.

TABLE 2

| | | | | HPLC | |
|---|---|---|---|---|---|
| | | | LCMS | method, | |
| Ex. | Structure | Name | (M + H)⁺ | RT(min) | ¹H NMR (ppm) |
| 2 | 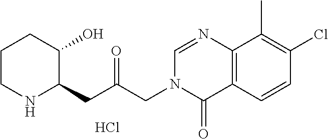 [SMILES: Cc1c(Cl)ccc2c(=O)n(CC(=O)C[C@H]3NCCC[C@@H]3O)cnc12 ; Cl] | 7-Chloro-3-{3- [(2R,3S)-3- hydroxypiper- idin-2-yl]-2- oxopropyl}-8- methylquin- azolin-4(3H)- one hydro- chloride | 350.3 | A:2.87 | (400 MHz, DMSO-d₆) δ 8.90-8.59 (m, 2H), 8.31 (s, 1H), 7.99 (d, J = 8.4 Hz, 1H), 7.62 (d, J = 8.4 Hz, 1H), 5.59 (br s, 1H), 5.10 (d, J = 18 Hz, 1H), 5.05 (d, J = 18 Hz, 1H), 3.52-3.46 (m, 1H), 3.30-3.13 (m, 3H), 2.94-2.88 (m, 2H), |
| | | | | | 2.61 (s, 3H), 1.93-1.90 (m, |
| | | | | | 1H), 1.80-1.76 (m, 1H), |
| | | | | | 1.65-1.62 (m, 1H), 1.50- |
| | | | | | 1.36 (m, 1H). |
| 3 | 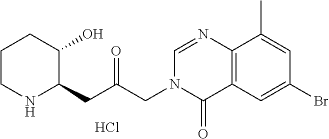 [SMILES: Cc1cc(Br)cc2c(=O)n(CC(=O)C[C@H]3NCCC[C@@H]3O)cnc12 ; Cl] | 6-Bromo-3-{3- [(2R,3S)-3- hydroxypiper- idin-2-yl]-2- oxopropyl}-8- methylquin- azolin-4(3H)- one hydro- chloride | 395.8 | A:2.71 | (400 MHz, DMSO-d₆) δ 9.29 (br s, 1H), 9.07 (br s, 1H), 8.33 (s, 1H), 8.06 (d, J = 2.0 Hz, 1H), 7.93 (d, J = 2.0 Hz 1H), 5.11 (s, 2H), 3.59-3.53 (m, 1H), 3.34-3.23 (m, 1H), 3.20- 3.12 (m, 2H), 2.97 (dd, J = 17.2, 6.0 Hz, 1H), 2.89- |
| | | | | | 2.82 (m, 1H), 2.55 (s, 3H), |
| | | | | | 1.96-1.90 (m, 1H), 1.81- |
| | | | | | 1.75 (m, 1H), 1.72-1.52 |
| | | | | | (m, 1H) 1.48-1.40 (m, 1H). |
| 4 | 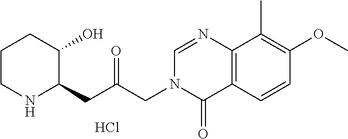 [SMILES: COc1ccc2c(=O)n(CC(=O)C[C@H]3NCCC[C@@H]3O)cnc2c1C ; Cl] | 3-{3-[(2R,3S)- 3-Hydroxy- piperidin-2-yl]- 2-oxopropyl}- 7-methoxy-8- methylquin- azolin-4(3H)- one hydro- chloride | 346.2 | A:2.38 | (400 MHz, DMSO-d₆: D₂O (~20:1 v/v)) δ 8.12 (s, 1H), 8.02 (d, J = 9.2 Hz, 1H), 7.30 (d, J = 8.8 Hz, 1H), 4.98 (s, 2H), 3.92 (s, 3H), 3.52-3.46 (m, 1H), 3.32- 3.29 (m, 1H), 3.28-3.12 (m, 2H), 2.91-2.83 (m, 2H), 2.36 (s, 3H), 1.95-1.88 (m, |
| | | | | | 1H), 1.87-1.80 (m, 1H), |
| | | | | | 1.67-1.55 (m, 1H), 1.48- |
| | | | | | 1.39 (m, 1H) |
| 5 | 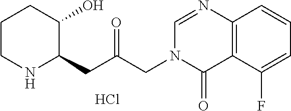 [SMILES: O=C(C[C@H]1NCCC[C@@H]1O)Cn1cnc2cccc(F)c2c1=O ; Cl] | 5-Fluoro-3-{3- [(2R,3S)-3- hydroxypiper- idin-2-yl]-2- oxopropyl}- quinazolin- 4(3H)-one | 320.2 | A:1.60 | (400 MHz, CD₃OD) δ 8.45 (s, 1H), 7.93-7.88 (m, 1H), 7.60-7.58 (m, 1H), 7.37- 7.32 (m, 1H), 5.13 (d, J = 17.6 Hz, 1H), 5.05 (d, J = 17.6 Hz, 1H), 3.72-3.61 (m, 1H), 3.51-3.39 (m, 2H), |
| | | hydrochloride | | | 3.32-3.30 (m, 1H), 3.12- |
| | | | | | 2.95 (m, 2H), 2.20-1.99 (m, |
| | | | | | 2H), 1.90-1.72 (m, 1H), |
| | | | | | 1.70-1.52 (m, 1H). |
| 6 | 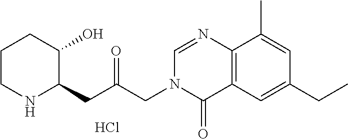 [SMILES: CCc1cc(C)c2ncn(CC(=O)C[C@H]3NCCC[C@@H]3O)c(=O)c2c1 ; Cl] | 6-Ethyl-3-(3- ((2R,3S)-3- hydroxypiper- idin-2-yl)-2- oxopropyl)-8- methylquin- azolin-4(3H)- one hydro- chloride | 344.3 | A:2.87 | (400 MHz, DMSO-d₆) δ 8.18 (d, J = 3.2 Hz, 1H), 7.79 (s, 1H), 7.61 (s, 1H), 5.23 (s, 1H), 5.02 (s, 2H), 3.11 (dd, J = 16.4, 4.4 Hz, 1H), 3.04-2.95 (m, 2H), 2.75-2.71 (m, 2H), 2.69- 2.68 (m, 2H), 2.53 (s, 3H), 1.98-1.88 (m, 2H), 1.75- |
| | | | | | 1.68 (m, 1H), 1.59-1.43 |
| | | | | | (m, 1H), 1.41-1.30 (m, 1H), |
| | | | | | 1.23 (d, J = 7.6 Hz, 3H). |
| 7 | 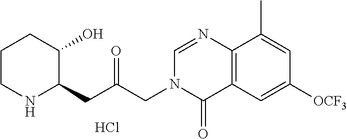 [SMILES: Cc1cc(OC(F)(F)F)cc2c(=O)n(CC(=O)C[C@H]3NCCC[C@@H]3O)cnc12 ; Cl] | 3-{3-[(2R,3S)- 3-Hydroxy- piperidin-2-yl]- 2-oxopropyl}- 8-methyl-6-(tri- fluoromethoxy)- quinazolin- 4(3H)-one hydrochloride | 400.2 | A:3.28 | (400 MHz, DMSO-d₆) δ 8.99-8.72 (m, 2H), 8.33 (s, 1H), 7.85-7.78 (m, 2H), 5.59 (d, J = 5.6 Hz, 1H), 5.11 (s, 2H), 3.57-3.47 (m, 1H), 3.3-3.28 (m, 1H), 3.27-3.18 (m, 1H), 3.17- 3.10 (m, 1H), 2.98-2.80 (m, 2H), 2.60 (s, 3H), 1.95- |
| | | | | | 1.86 (m, 1H), 1.82-1.72 (m, |
| | | | | | 1H), 1.70-1.58 (m, 1H), |
| | | | | | 1.50-1.38 (m, 1H). |
| 8 | 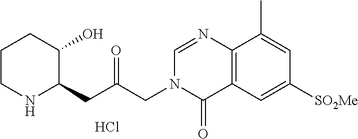 [SMILES: Cc1cc(S(C)(=O)=O)cc2c(=O)n(CC(=O)C[C@H]3NCCC[C@@H]3O)cnc12 ; Cl] | 3-{3-[(2R,3S)- 3-Hydroxy- piperidin-2-yl]- 2-oxopropyl}- 8-methyl-6- methylsulfonyl)- quinazolin- 4(3H)-one hydrochloride | 394.2 | G:5.75 | (400 MHz, CD₃OD) δ 8.61 (d, J = 2.0 Hz, 1H), 8.33 (s, 1H), 8.21 (d, J = 2.0 Hz, 1H), 5.13 (d, J = 17.6 Hz, 1H), 5.05 (d, J = 17.6 Hz, 1H), 3.70-3.60 (m, 1H), 3.52-3.43 (m, 2H), 3.41- 3.35 (m, 1H), 3.20 (s, 3H), 3.11-3.00 (m, 2H), 2.71 (s, |
| | | | | | 3H), 2.15-2.03 (m, 2H), |
| | | | | | 1.85-1.70 (m, 1H), 1.68- |
| | | | | | 1.52 (m, 1H). |
| 9 | 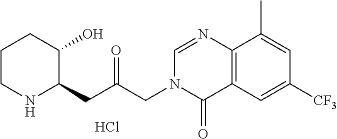 [SMILES: Cc1cc(C(F)(F)F)cc2c(=O)n(CC(=O)C[C@H]3NCCC[C@@H]3O)cnc12 ; Cl] | 3-{3-[(2R,3S)- 3-Hydroxy- piperidin-2-yl]- 2-oxopropyl}- 8-methyl-6-(tri- fluoromethyl)- quinazolin- 4(3H)-one hydrochloride | 384.2 | A:3.09 | (400 MHz, CD₃OD) δ 8.35 (s, 1H), 8.28 (s, 1H), 7.98 (s, 1H), 5.13 (d, J = 17.6 Hz, 1H), 5.04 (d, J = 17.6 Hz, 1H), 3.71-3.62 (m, 1H), 3.51-3.36 (m, 3H), 3.12-3.00 (m, 2H), 2.70 (s, 3H), 2.19-2.02 (m, 2H), 1.85-1.70 (m, 1H), 1.68- |
| | | | | | 1.52 (m, 1H). |
| 10 | 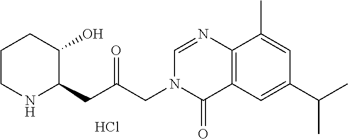 [SMILES: Cc1cc(C(C)C)cc2c(=O)n(CC(=O)C[C@H]3NCCC[C@@H]3O)cnc12 ; Cl] | 3-{3-[(2R,3S)- 3-Hydroxy- piperidin-2-yl]- 2-oxopropyl}- 6-isopropyl-8- methylquin- azolin-4(3H)- one hydro- chloride | 358.2 | A:3.176 | (400 MHz, DMSO-d₆) δ 8.72 (br s, 2H), 8.19 (s, 1H), 7.81 (d, J = 1.9 Hz, 1H), 7.67 (s, 1H), 5.05 (s, 2H), 3.49-3.31 (m, 1H), 3.26-3.21 (m, 1H), 3.19- 3.15 (m, 1H), 3.04-3.01 (m, 1H), 2.91-2.87 (m, 3H), 2.55 (s, 3H), 1.94- |
| | | | | | 1.91 (m, 1H), 1.85-1.80 |
| | | | | | (m, 1H), 1.67-1.58 (m, |
| | | | | | 1H), 1.48-1.41 (m, 1H), |
| | | | | | 1.34 (d, J = 6.8 Hz, 6H). |
| 11 | 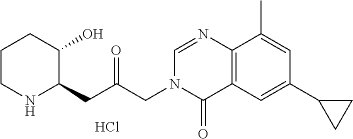 [SMILES: Cc1cc(C2CC2)cc2c(=O)n(CC(=O)C[C@H]3NCCC[C@@H]3O)cnc12 ; Cl] | 6-Cyclopropyl- 3-{3-[(2R,3S)- 3-hydroxypiper- idin-2-yl]-2- oxopropyl}-8- methylquin- azolin-4(3H)- one hydro- chloride | 356.2 | C: 4.66 | (400 MHz, DMSO-d₆) δ 9.15 (br s, 1H), 8.97 (br s, 1H), 8.19 (s, 1H), 7.65 (d, J = 1.2 Hz, 1H), 7.43 (s, 1H), 5.06 (s, 2H), 3.56- 3.52 (m, 1H), 3.29-3.21 (m, 1H), 3.18-3.12 (m, 2H), 2.97-2.85 (m, 2H), 2.55 (s, 3H), 2.08-2.04 (m, 1H), |
| | | | | | 1.93-1.90 (m, 2H), 1.78- |
| | | | | | 1.75 (m, 1H), 1.46-1.43 |
| | | | | | (m, 1H), 1.04-0.97 (m, 2H), |
| | | | | | 0.89-0.76 (m, 2H). |
| 12 | 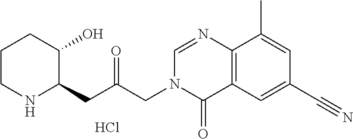 [SMILES: Cc1cc(C#N)cc2c(=O)n(CC(=O)C[C@H]3NCCC[C@@H]3O)cnc12 ; Cl] | 3-{3-[(2R,3S)- 3-Hydroxy- piperidin-2-yl]- 2-oxopropyl}- 8-methyl-4- oxo-3,4-di- hydroquinazol- ine-6-carboni- trile hydro- | 341.2 | C: 5.519 | (400 MHz, DMSO-d₆: D₂O [~20:1 v/v]) δ 8.39 (d, J = 1.6 Hz, 1H), 8.37 (s, 1H), 8.12 (d, J = 1.2 Hz, 1H), 5.09 (m, 2H), 3.61-3.48 (m, 1H), 3.28-3.14 (m, 3H), 2.93-2.86 (m, 2H), 2.60 (s, 3H), 1.96-1.92 (m, 1H), 1.86-1.82 (m, 1H), 1.63- |
| | | chloride | | | 1.51 (m, 1H), 1.47-1.44 |
| | | | | | (m, 1H). |
| 13 | 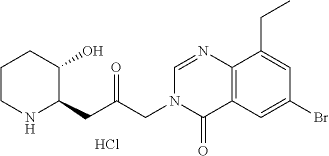 [SMILES: CCc1cc(Br)cc2c(=O)n(CC(=O)C[C@H]3NCCC[C@@H]3O)cnc12 ; Cl] | 6-Bromo-8- ethyl-3-{3- [(2R,3S)-3- hydroxypiper- idin-2-yl]-2- oxopropyl}- quinazolin- 4(3H)-one hydrochloride | 401.1 | A:3.14 | (400 MHz, DMSO-d₆) δ 8.79 (br s, 2H), 8.28 (d, J = 2.0 Hz, 1H), 8.07 (s, 1H), 7.91 (s, 1H), 5.58 (d, J = 5.2 Hz, 1H), 5.08 (s, 2H), 3.52-3.48 (m, 1H), 3.25-3.13 (m, 3H), 3.03- 2.98 (m, 2H), 2.97-2.87 (m, 2H), 1.94-1.90 (m, 1H), 1.82-1.79 (m, 1H), |
| | | | | | 1.68-1.59 (m, 1H), 1.50- |
| | | | | | 1.43 (m, 1H), 1.25-1.21 |
| | | | | | (m, 3H). |
| 14 | 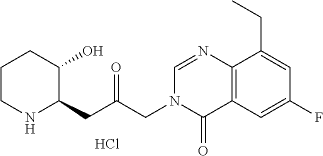 [SMILES: CCc1cc(F)cc2c(=O)n(CC(=O)C[C@H]3NCCC[C@@H]3O)cnc12 ; Cl] | 8-Ethyl-6- fluoro-3-(3- ((2R,3S)-3- hydroxypiper- idin-2-yl)-2- oxopropyl)- quinazolin- 4(3H)-one hydrochloride | 348.1 | C: 6.56 | (400 MHz, DMSO-d₆) δ 8.77 (br s, 2H), 8.23 (s, 1H), 7.66 (d, J = 3.2 Hz, 1H), 7.64 (s, 1H), 5.57 (d, J = 5.2 Hz, 1H), 5.07 (s, 2H), 3.55-3.48 (m, 1H), 3.28-3.19 (m, 1H), 3.17- 3.12 (m, 2H), 3.03 (q, J = 7.2 Hz, 2H), 2.93-2.87 (m, 2H), 1.97-1.88 (m, 1H), |
| | | | | | 1.84-1.75 (m, 1H), 1.68- |
| | | | | | 1.58 (m, 1H), 1.52-1.40 |
| | | | | | (m, 1H), 1.24 (t, J = 7.6 |
| | | | | | 7.6 Hz, 3H). |
| 15 | 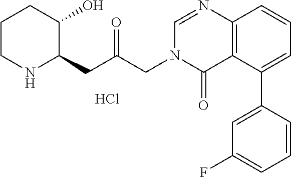 [SMILES: O=C(C[C@H]1NCCC[C@@H]1O)Cn1cnc2cccc(-c3cccc(F)c3)c2c1=O ; Cl] | 5-(3-Fluoro- phenyl)-3-{3- [(2R,3S)-3- hydroxypiper- idin-2-yl]-2- oxopropyl}- quinazolin- 4(3H)-one hydrochloride | 396.9 | C:4.45 | (400 MHz, DMSO-d₆) δ 8.85-8.62 (m, 2H), 8.30 (s, 1H), 7.86 (t, J = 7.6 Hz, 1H), 7.77 (d, J = 1.2 Hz, 1H), 7.40-7.29 (m, 2H), 7.17-7.08 (m, 3H), 4.95 (s, 2H), 3.50-3.42 (m, 1H), 3.31-3.20 (m, 1H), 3.19- 3.06 (m, 2H), 2.91-2.75 (m, 2H), 1.92-1.85 (m, 1H), 1.84-1.72 (m, 1H), |
| | | | | | 1.69-1.52 (m, 1H), 1.38- |
| | | | | | 1.32 (m, 1H). |
| 16 | 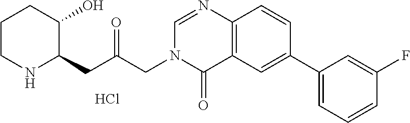 [SMILES: O=C(C[C@H]1NCCC[C@@H]1O)Cn1cnc2ccc(-c3cccc(F)c3)cc2c1=O ; Cl] | 6-(3-Fluoro- phenyl)-3-{3- [(2R,3S)-3- hydroxypiper- idin-2-yl]-2- oxopropyl}- quinazolin- 4(3H)-one | 396.0 | G:8.67 | (400 MHz, DMSO-d₆) δ 9.00-8.80 (m, 2H), 8.37 (d, J = 2.4 Hz, 1H), 8.27 (s, 1H), 8.22 (dd, J = 8.4, 2.4 Hz, 1H), 7.82 (d, J = 8.8 Hz, 1H), 7.68-7.6 (m, 2H), 7.59-7.52 (m, 1H), 7.30- 7.24 (m, 1H), 5.12 (s, 2H), |
| | | hydrochloride | | | 3.52-3.43 (m, 1H), 3.46- |
| | | | | | 3.13 (m, 3H), 2.98-2.83 |
| | | | | | (m, 2H), 1.99-1.90 (m, 1H), |
| | | | | | 1.89-1.72 (m, 1H), 1.72- |
| | | | | | 1.55 (m, 1H), 1.52-1.44 |
| | | | | | (m, 1H). |
| 17 | 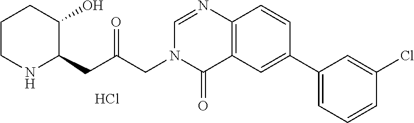 [SMILES: O=C(C[C@H]1NCCC[C@@H]1O)Cn1cnc2ccc(-c3cccc(Cl)c3)cc2c1=O ; Cl] | 6-(3-Chloro- phenyl)-3-{3- [(2R,3S)-3- hydroxypiper- idin-2-yl]-2- oxopropyl}- quinazolin- 4(3H)-one | 412.2 | E:7.33 | (400 MHz, DMSO-d₆) δ 9.10 (br s, 1H), 8.94 (br s, 1H), 8.36 (d, J = 2.4 Hz, 1H), 8.28 (s, 1H), 8.22 (dd, J = 8.6, 2.4 Hz, 1H), 7.83-7.79 (m, 2H), 7.75 (d, J = 8.6 Hz, 1H), 7.55 (t, J = 8.0 Hz, 1H), 7.49 (d, |
| | | hydrochloride | | | J = 8.0 Hz, 1H), 5.12 (s, |
| | | | | | 2H), 3.60-3.52 (m, 1H), |
| | | | | | 3.32-3.10 (m, 3H), 3.01- |
| | | | | | 2.82 (m, 2H), 1.94-1.90 |
| | | | | | (m, 1H), 1.85-1.78 (m, |
| | | | | | 1H), 1.72-1.63 (m, 1H), |
| | | | | | 1.55-1.38 (m, 1H). |
| 18 | 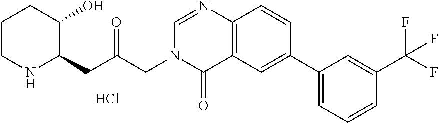 [SMILES: O=C(C[C@H]1NCCC[C@@H]1O)Cn1cnc2ccc(-c3cccc(C(F)(F)F)c3)cc2c1=O ; Cl] | 3-{3-[(2R,3S)- 3-Hydroxy- piperidin-2-yl]- 2-oxopropyl}- 6-[3-(trifluoro- methyl)phenyl]- quinazolin- 4(3H)-one | 446.0 | C:7.48 | (400 MHz, DMSO-d₆) δ 8.79 (s, 2H), 8.40 (d, J = 2.0 Hz, 1H), 8.29 (d, J = 2.0 Hz, 1H), 8.26 (d, J = 2.8 Hz, 1H), 8.17-8.10 (m, 2H), 7.86-7.78 (m, 3H), 5.61 (br s, 1H), 5.15 (s, 2H), 3.59-3.49 (m, 1H), |
| | | hydrochloride | | | 3.26-3.20 (m, 1H), 3.19- |
| | | | | | 3.10 (m, 2H), 2.99-2.82 |
| | | | | | (m, 2H), 1.94-1.89 (m, |
| | | | | | 1H), 1.86-1.77 (m, 1H), |
| | | | | | 1.68-1.57 (m, 1H), 1.51- |
| | | | | | 1.40 (m, 1H). |
| 19 | 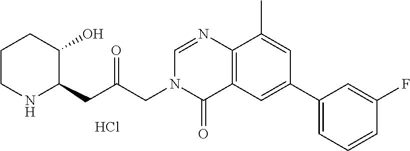 [SMILES: Cc1cc(-c2cccc(F)c2)cc2c(=O)n(CC(=O)C[C@H]3NCCC[C@@H]3O)cnc12 ; Cl] | 6-(3-Fluoro- phenyl)-3-{3- [(2R,3S)-3- hydroxypiper- idin-2-yl]-2- oxopropyl}-8- methylquin- azolin-4(3H)- one hydro- chloride | 410.2 | C:6.31 | (400 MHz, DMSO-d₆) δ 9.18 (br s, 1H), 8.99 (br s, 1H), 8.32 (s, 1H), 8.22 (s, 1H), 8.12 (s, 1H), 7.76- 7.49 (m, 3H), 7.31-7.29 (m, 1H), 5.13 (s, 2H), 3.59-3.50 (m, 1H), 3.37-3.09 (m, 3H), 3.05-2.80 (m, 2H), 2.67 (s, 3H), 2.01-1.83 (m, 1H), 1.82-1.73 (m, 1H), 1.72- |
| | | | | | 1.56 (m, 1H), 1.52-1.32 |
| | | | | | (m, 1H). |
| 20 | 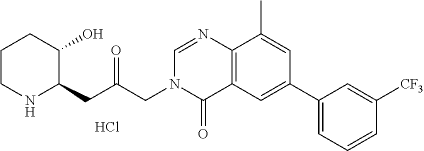 [SMILES: Cc1cc(-c2cccc(C(F)(F)F)c2)cc2c(=O)n(CC(=O)C[C@H]3NCCC[C@@H]3O)cnc12 ; Cl] | 3-{3-[(2R,3S)- 3-Hydroxy- piperidin-2-yl]- 2-oxopropyl}- 8-methyl-6-[3- (trifluoro- methyl)phenyl]- quinazolin- 4(3H)-one hydrochloride | 460.4 | C:7.39 | (400 MHz, DMSO-d₆) δ 9.08-8.81 (m, 2H), 8.31 (s, 1H), 8.26 (d, J = 2.0 Hz, 1H), 8.19 (d, J = 1.6 Hz, 1H), 8.13-8.05 (m, 2H), 7.81-7.73 (m, 2H), 5.12 (s, 2H), 3.58-3.47 (m, 2H), 3.37-3.28 (m, 1H), 3.27- 3.21 (m, 1H), 3.20-3.17 (m, 1H), 2.99 (dd, J = |
| | | | | | 17.0, 6.4 Hz, 1H), 2.65 (s, |
| | | | | | 3H), 1.95-1.89 (m, 1H), |
| | | | | | 1.87-1.73 (m, 1H), 1.72- |
| | | | | | 1.60 (m, 1H), 1.50-1.40 |
| | | | | | (m, 1H). |
| 21 | 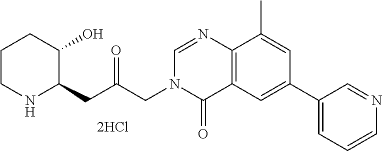 [SMILES: Cc1cc(-c2cccnc2)cc2c(=O)n(CC(=O)C[C@H]3NCCC[C@@H]3O)cnc12 ; "2HCl"] | 3-{3-[(2R,3S)- 3-Hydroxy- piperidin-2-yl]- 2-oxopropyl}- 8-methyl-6- (pyridin-3-yl)- quinazolin- 4(3H)-one dihydrochloride | 393.3 | C:4.31 | (400 MHz, DMSO-d₆) δ 9.32 (br s, 2H), 9.16-9.12 (m, 1H), 8.88-8.84 (m, 2H), 8.41-8.38 (m, 2H), 8.27-8.24 (m, 1H), 8.06- 8.02 (m, 1H), 5.16 (s, 2H), 3.74-3.43 (m, 2H), 3.37-3.28 (m, 1H), 3.25- 3.18 (m, 1H), 3.17-3.10 (m, 1H), 2.99 (dd, J = |
| | | | | | 17.0, 6.4 Hz, 1H), 2.65 (s, |
| | | | | | 3H), 1.98-1.88 (m, 1H), |
| | | | | | 1.85-1.62 (m, 2H), 1.52- |
| | | | | | 1.39 (m, 1H). |
| 22 | 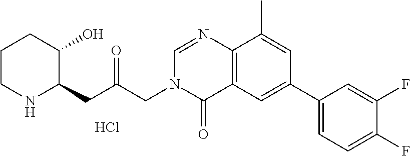 [SMILES: Cc1cc(-c2ccc(F)c(F)c2)cc2c(=O)n(CC(=O)C[C@H]3NCCC[C@@H]3O)cnc12 ; Cl] | 6-(3,4-Difluoro- phenyl)-3-{3- [(2R,3S)-3- hydroxypiper- idin-2-yl]-2- oxopropyl}-8- methylquin- azolin-4(3H)- one hydro- chloride | 428.0 | C:6.77 | (400 MHz, DMSO-d₆) δ 8.63 (br s, 1H), 8.54 (br s, 1H), 8.24 (s, 1H), 8.19 (d, J = 2.0 Hz, 1H), 8.10 (d, J = 1.2 Hz, 1H), 7.92-7.86 (m, 1H), 7.65-7.64 (m, 1H), 7.63-7.57 (m, 1H), 5.58 (d, J = 4.4 Hz, 1H), 5.08 (s, 2H), 3.48-3.43 (m, 1H), 3.27-3.21 (m, 2H), |
| | | | | | 3.18-2.93 (m, 1H), 2.91- |
| | | | | | 2.80 (m, 2H), 2.62 (s, 3H), |
| | | | | | 1.95-1.91 (m, 1H), 1.85- |
| | | | | | 1.81 (m, 1H), 1.66-1.52 |
| | | | | | (m, 1H), 1.50-1.45 (m, 1H). |
| 23 | 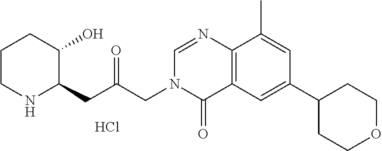 [SMILES: Cc1cc(C2CCOCC2)cc2c(=O)n(CC(=O)C[C@H]3NCCC[C@@H]3O)cnc12 ; Cl] | 3-{3-[(2R,3S)- 3-Hydroxy- piperidin-2-yl]- 2-oxopropyl}- 8-methyl-6- (tetrahydro-2H- pyran-4-yl)- quinazolin- 4(3H)-one hydrochloride | 400.2 | C:5.17 | (400 MHz, DMSO-d₆) δ 9.20 (br s, 1H), 8.98 (br s, 1H), 8.23 (s, 1H), 7.80 (d, J = 2.0 Hz, 1H), 7.67 (d, J = 1.2 Hz, 1H), 5.08 (s, 2H), 3.99-3.95 (m, 2H), 3.61-3.40 (m, 3H), 3.29- 3.23 (m, 1H), 3.21-3.20 (m, 1H), 3.18-3.08 (m, 1H), 2.99-2.81 (m, 3H), |
| | | | | | 2.55 (s, 3H), 1.98-1.87 (m, |
| | | | | | 1H), 1.85-1.61 (m, 6H), |
| | | | | | 1.50-1.38 (m, 1H). |
| 24 | 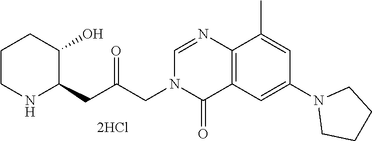 [SMILES: Cc1cc(N2CCCC2)cc2c(=O)n(CC(=O)C[C@H]3NCCC[C@@H]3O)cnc12 ; "2HCl"] | 3-{3-[(2R,3S)- 3-Hydroxy- piperidin-2-yl]- 2-oxopropyl}- 8-methyl-6- (pyrrolidin-1- yl)quinazolin- 4(3H)-one dihydrochloride | 385.3 | C:5.04 | (400 MHz, DMSO-d₆) δ 8.59 (br s, 2H), 7.92 (s, 1H), 7.05 (s, 1H), 6.87 (d, J = 1.6 Hz, 1H), 5.60 (br s, 1H), 5.01 (d, J = 17.6 Hz, 1H), 4.95 (d, J = 17.6 Hz, 1H), 3.48-3.44 (m, 1H), 3.31-3.23 (m, 5H), 3.20-3.14 (m, 2H), 2.92- |
| | | | | | 2.81 (m, 2H), 2.54 (s, 3H), |
| | | | | | 1.99-1.90 (m, 5H), 1.87- |
| | | | | | 1.79 (m, 1H), 1.64-1.53 (m, |
| | | | | | 1H), 1.50-1.40 (m, 1H). |
| 25 | 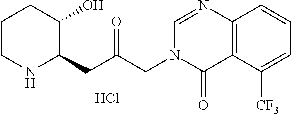 [SMILES: O=C(C[C@H]1NCCC[C@@H]1O)Cn1cnc2cccc(C(F)(F)F)c2c1=O ; Cl] | 3-{3-[(2R,3S)- 3-Hydroxy- piperidin-2-yl]- 2-oxopropyl}- 5-(trifluoro- methyl)quin- azolin-4(3H)- | 370.0 | E:4.63 | (400 MHz, CD₃OD) δ 8.30 (s, 1H), 8.02-7.96 (m, 3H), 5.13 (d, J = 17.6 Hz, 1H), 5.13 (d, J = 17.6 Hz, 1H), 3.70-3.55 (m, 1H), 3.50- 3.35 (m, 2H), 3.32-3.27 (m, 1H), 3.12-2.92 (m, |
| | | one hydro- | | | 2H), 2.16-1.98 (m, 2H), |
| | | chloride | | | 1.80-1.62 (m, 1H), 1.65- |
| | | | | | 1.50 (m, 1H). |
| 26 | 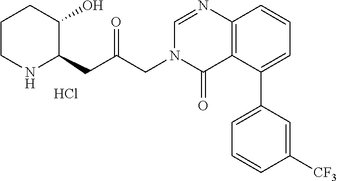 [SMILES: O=C(C[C@H]1NCCC[C@@H]1O)Cn1cnc2cccc(-c3cccc(C(F)(F)F)c3)c2c1=O ; Cl] | 3-{3-[(2R,3S)- 3-Hydroxy- piperidin-2-yl]- 2-oxopropyl}- 5-[3-(trifluoro- methyl)phenyl]- quinazolin- 4(3H)-one hydrochloride | 446.1 | G:9.75 | (400 MHz, DMSO-d₆) δ 9.16 (br s, 1H), 8.93 (br s, 1H), 8.31 (s, 1H), 7.88 (t, J = 7.6 Hz, 1H), 7.78 (dd, J = 8.2, 1.2 Hz, 1H), 7.72- 7.66 (m, 1H), 7.60-7.57 (m, 3H), 7.35 (dd, J = 7.2, 0.8 Hz, 1H), 4.99 (s, 2H), 3.53-3.48 (m, 1H), 3.27- 3.18 (m, 1H), 3.14-3.08 (m, 2H), 2.90-2.80 (m, |
| | | | | | 2H), 1.91-1.86 (m, 1H), |
| | | | | | 1.79-1.73 (m, 1H), 1.70- |
| | | | | | 1.59 (m, 1H), 1.43-1.35 |
| | | | | | (m, 1H). |
| 27 | 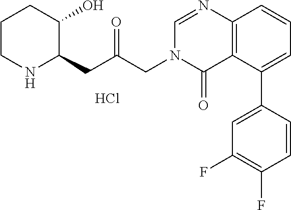 [SMILES: O=C(C[C@H]1NCCC[C@@H]1O)Cn1cnc2cccc(-c3ccc(F)c(F)c3)c2c1=O ; Cl] | 5-(3,4-Difluoro- phenyl)-3-{3- [(2R,3S)-3- hydroxypiper- idin-2-yl]-2- oxopropyl}- quinazolin- 4(3H)-one hydrochloride | 414.0 | E:6.72 | (400 MHz, DMSO-d₆) δ 8.70 (br s, 2H), 8.25 (s, 1H), 7.86 (t, J = 7.6 Hz, 1H), 7.76 (d, J = 8.4 Hz, 1H), 7.41-7.32 (m, 3H), 7.13-7.08 (m, 1H), 4.95 (d, J = 17.6 Hz, 1H), 4.93 (d, J = 17.6 Hz, 1H), 3.31- 3.21 (m, 2H), 3.19-3.08 (m, 2H), 2.92-2.72 (m, 2H), 1.95-1.85 (m, 1H), 1.81-1.70 (m, 1H), 1.65- 1.50 (m, 1H), 1.48-1.34 |
| | | | | | (m, 1H). |
| 28 | 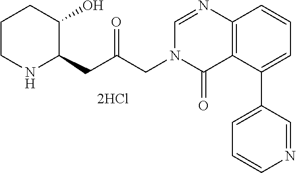 [SMILES: O=C(C[C@H]1NCCC[C@@H]1O)Cn1cnc2cccc(-c3cccnc3)c2c1=O ; "2HCl"] | 3-{3-[(2R,3S)- 3-Hydroxy- piperidin-2-yl]- 2-oxopropyl}- 5-(pyridin-3- yl)quinazolin- 4(3H)-one dihydrochloride | 379.2 | H:8.91 | (400 MHz, DMSO-d₆) δ 9.25 (br s, 1H), 8.90 (br s, 1H), 8.93 (s, 1H), 8.85 (d, J = 5.6 Hz, 1H), 8.47 (d, J = 7.6 Hz, 1H), 8.35 (s, 1H), 8.02-7.98 (m, 2H), 7.97-7.88 (m, 1H), 7.51 (t, J = 6.0 Hz, 1H), 5.00 (s, 2H), 3.29-3.20 (m, 1H), 3.17-3.05 (m, 3H), 2.91- 2.79 (m, 2H), 1.90-1.87 |
| | | | | | (m, 1H), 1.83-1.75 (m, |
| | | | | | 1H), 1.71-1.60 (m, 1H), |
| | | | | | 1.48-1.35 (m, 1H). |
| 29 | 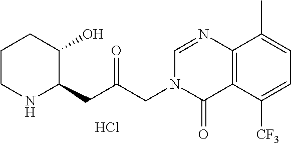 [SMILES: Cc1ccc(C(F)(F)F)c2c(=O)n(CC(=O)C[C@H]3NCCC[C@@H]3O)cnc12 ; Cl] | 3-{3-[(2R,3S)- 3-Hydroxy- piperidin-2-yl]- 2-oxopropyl}- 8-methyl-5-(tri- fluoromethyl)- quinazolin- 4(3H)-one hydrochloride | 384.2 | E:5.04 | (400 MHz, DMSO-d₆) δ 9.06 (br s, 1H), 8.91 (br s, 1H), 8.40 (s, 1H), 7.88 (s, 2H), 5.11 (s, 2H), 3.30- 3.08 (m, 4H), 3.00-2.80 (m, 2H), 2.67 (s, 3H), 1.97-1.87 (m, 1H), 1.86- 1.74 (m, 1H), 1.73-1.60 (m, 1H), 1.50-1.38 (m, 1H). |

### Intermediate 5: Tert-butyl (2R,3S)-2-{(S)-3-[6-chloro-8-methyl-4-oxoquinazolin-3(4H)-yl]-2-hydroxypropyl}-3-hydroxypiperidine-1-carboxylate

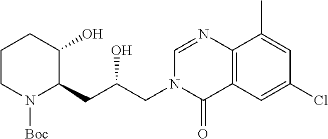 [SMILES: Cc1cc(Cl)cc2c(=O)n(C[C@@H](O)C[C@@H]3[C@@H](O)CCCN3C(=O)OC(C)(C)C)cnc12]

Tert-butyl (2R,3S)-2-[3-(6-chloro-8-methyl-4-oxo-quinazolin-3-yl)-2-oxo-propyl]-3-hydroxy-piperidine-1-carboxylate (450 mg, 1.0 mmol) was suspended in ethanol (10 mL) and water (2.5 mL) at −10° C. NaBH₄ (18.3 mg, 0.47 mmol) was added and stirred for additional 30 min at the same temperature. The reaction mixture was diluted with EtOAc (50 mL), washed with water (25 mL). The organic layer was washed with brine solution (20 mL), dried over Na₂SO₄ and concentrated to get crude product as an off-white solid. The crude product was purified by HPLC on symmetry C8 column using water/MeCN as mobile phase to get title compound (130 mg, 29%) as a white solid (The first peak arbitrarily assigned as title compound based on literature, US20150057297 A1). LCMS (ESI) m/z: 452.3 [M+H]⁺; ¹H NMR (400 MHz, CD₃OD) δ 8.26 (s, 1H), 8.05 (d, J=2.4 Hz, 1H), 7.68 (dd, J=2.4, 0.8 Hz, 1H), 4.46 (t, J=7.2 Hz, 1H), 4.27 (d, J=10.8 Hz, 1H), 4.05-3.80 (m, 3H), 3.83-3.78 (m, 1H), 2.98-2.86 (m, 1H), 2.60 (s, 3H), 1.98-1.69 (m, 5H), 1.50 (s, 9H), 1.43-1.37 (m, 1H).

The diastereomer tert-butyl (2R,3S)-2-[(2R)-3-(6-chloro-8-methyl-4-oxo-quinazolin-3-yl)-2-hydroxy-propyl]-3-hydroxy-piperidine-1-carboxylate, second peak, also isolated (96 mg, 17.5%) as a white solid. LCMS (ESI) m/z: 452.3 [M+H]⁺; ¹H NMR (400 MHz, CD₃OD) δ 8.25 (s, 1H), 8.02 (d, J=2.4 Hz, 1H), 7.66 (dd, J=2.4, 0.8 Hz, 1H), 4.38-4.44 (m, 1H), 4.32 (dd, J=13.2, 2.8 Hz, 1H), 4.03-3.95 (m, 1H), 3.93-3.83 (m, 1H), 3.81-3.74 (m, 2H), 2.97-2.82 (m, 1H), 2.59 (s, 3H), 2.02-1.82 (m, 2H), 1.80-1.69 (m, 2H), 1.53-1.48 (m, 1H), 1.44 (s, 9H), 1.41-1.38 (m, 1H).

### Example 30: 6-Chloro-3-{(S)-2-hydroxy-3-[(2R,3S)-3-hydroxypiperidin-2-yl]propyl}-8-methylquinazolin-4(3H)-one hydrochloride

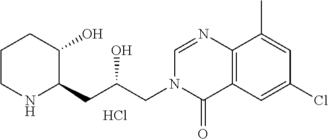 [SMILES: Cc1cc(Cl)cc2c(=O)n(C[C@@H](O)C[C@H]3NCCC[C@@H]3O)cnc12 ; Cl]

To a solution of tert-butyl (2R,3S)-2-[(2S)-3-(6-chloro-8-methyl-4-oxo-quinazolin-3-yl)-2-hydroxy-propyl]-3-hydroxy-piperidine-1-carboxylate (130 mg, 0.29 mmol) in CH₂CO₂ (15 mL) was added 4 M HCl in dioxane (2 mL) at 0° C. under nitrogen atmosphere and the reaction mixture was stirred at rt for 1 h. The reaction mixture was concentrated to get the crude product as an off-white solid. The solids were triturated with diethyl ether (5 mL) to get 6-chloro-3-{(S)-2-hydroxy-3-[(2R,3S)-3-hydroxypiperidin-2-yl]propyl}-8-methylquinazolin-4(3H)-one hydrochloride (89 mg, 86%) as a white solid. LCMS (ESI) m/z: 352.3 [M+H]⁺; ¹H NMR (400 MHz, DMSO-d₆) δ 8.78 (br s, 1H), 8.35 (s, 1H), 8.27 (br s, 1H), 7.96 (d, J=2.1 Hz, 1H), 7.80 (d, J=1.6 Hz, 1H), 4.13-4.10 (m, 2H), 3.90-3.88 (m, 1H), 3.49-3.45 (m, 1H), 3.16-3.12 (m, 1H), 3.03-2.95 (m, 1H), 2.83-2.78 (m, 1H), 2.55 (s, 3H), 2.16-2.12 (m, 1H), 1.92-1.88 (m, 1H), 1.80-1.77 (m, 1H), 1.62-1.37 (m, 3H). Analytical HPLC RT=2.65 min (Method A).

The following Examples in Table 3 were prepared by using the same procedure as described in Example 30 by de-protection of the corresponding Boc intermediate.

TABLE 3

| | | | | HPLC | |
|---|---|---|---|---|---|
| | | | LCMS | method, | |
| Ex. | Structure | Name | (M + H)⁺ | RT(min) | ¹H NMR (ppm) |
| 31 | 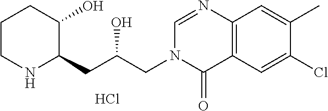 [SMILES: Cc1cc2ncn(C[C@@H](O)C[C@H]3NCCC[C@@H]3O)c(=O)c2cc1Cl ; Cl] | 6-Chloro-3-{(S)- 2-hydroxy-3-[(2R, 3S)-3-hydroxy- piperidin-2-yl]- propyl}-7-methyl- quinazolin-4(3H)- one hydrochloride | 352.3 | A: 2.55 | (400 MHz, CD₃OD) δ 8.47 (s, 1H), 8.25 (s, 1H), 7.68 (s, 1H), 4.31-4.28 (m, 2H), 3.98-3.95 (m, 1H), 3.75- 3.57 (m, 1H), 3.35-3.32 (m, 1H), 3.14-3.10 (m, 1H), 3.01-2.98 (m, 1H), 2.57 (s, |
| | | | | | 3H), 2.39-2.36 (m, 1H), |
| | | | | | 1.88-1.99 (m, 2H), 1.62- |
| | | | | | 1.56 (m, 3H). |
| 32 | 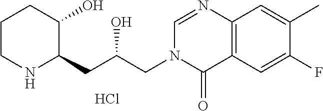 [SMILES: Cc1cc2ncn(C[C@@H](O)C[C@H]3NCCC[C@@H]3O)c(=O)c2cc1F ; Cl] | 6-Fluoro-3-{(S)- 2-hydroxy-3-[(2R, 3S)-3-hydroxy- piperidin-2-yl]- propyl}-7-methyl- quinazolin-4(3H)- one hydrochloride | 336.2 | A:2.24 | (400 MHz, DMSO-d₆): δ 9.11 (br s, 1H), 8.38 (br s, 1H), 8.34 (s, 1H), 7.78 (d, J = 9.2 Hz, 1H), 7.68 (d, J = 6.4 Hz, 1H), 4.14-4.11 (m, 2H), 3.89-3.83 (m, 1H), 3.52-3.47 (m, 1H), 3.14- |
| | | | | | 3.11 (m, 1H), 3.01-2.91 |
| | | | | | (m, 1H), 2.86-2.75 (m, |
| | | | | | 1H), 2.41 (s, 3H), 2.16- |
| | | | | | 2.12 (m, 1H), 1.92-1.88 |
| | | | | | (m, 1H), 1.79-1.76 (m, |
| | | | | | 1H), 1.66-1.55 (m, 2H), |
| | | | | | 1.41-1.38 (m, 1H). |
| 33 | 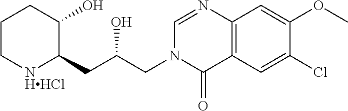 | 6-Chloro-3-{(S)- 2-hydroxy-3-[(2R, 3S)-3-hydroxy- piperidin-2-yl]- propyl}-7-meth- oxyquinazolin- 4(3H)-one | 368.3 | A:2.39 | (400 MHz,DMSO-d₆) δ 9.00-8.73 (m, 1H), 8.38- 8.22 (m, 2H), 8.10 (s, 1H), 7.32 (s, 1H), 5.70 (s, 1H), 5.44 (d, J = 5.2 Hz, 1H), 4.18-4.10 (m, 2H), 4.01 (s, 3H), 3.85 (dd, J = 14.0, |
| | | hydrochloride | | | 8.2 Hz, 1H), 3.53-3.42 (m, |
| | | | | | 1H), 3.20-3.12 (m, 1H), |
| | | | | | 3.05-2.93 (m, 1H), 2.83- |
| | | | | | 2.74 (m, 1H), 2.18-2.10 |
| | | | | | (m, 1H), 1.95-1.85 (m, |
| | | | | | 1H), 1.77-1.80 (m, 1H), |
| | | | | | 1.48-1.68 (m, 2H), 1.35- |
| | | | | | 1.45 (m, 1H). |
| 34 | 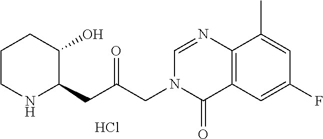 [SMILES: Cc1cc(F)cc2c(=O)n(CC(=O)C[C@H]3NCCC[C@@H]3O)cnc12 ; Cl] | 6-Fluoro-3-{(S)- 2-hydroxy-3-[(2R, 3S)-3-hydroxy- piperidin-2-yl]- propyl}-8-methyl- quinazolin-4(3H)- one hydrochloride | 336.2 | B:3.94 | (400 MHz, DMSO-d₆) δ 8.95 (br s, 1H), 8.86 (s, 1H), 8.30 (br s, 2H), 7.67 (d, J = 6.0 Hz, 1H), 7.65 (d, J = 6.4 Hz, 1H), 4.26- 4.11 (m, 2H), 3.90-3.81 (m, 1H), 3.48 (td, J = 13.5, 4.0 Hz, 1H), 3.18-3.11 (m, 1H), 3.02-2.93 (m, 1H), |
| | | | | | 2.83-2.72 (m, 1H), 2.68- |
| | | | | | 2.50 (m, 1H), 2.57 (s, 3H), |
| | | | | | 1.94-1.86 (m, 1H), 1.83- |
| | | | | | 1.74 (m, 1H), 1.68-1.49 (m, |
| | | | | | 2H), 1.47-1.32 (m, 1H). |
| 35 | 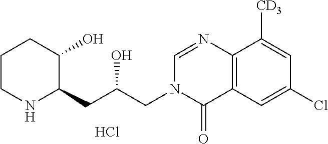 [SMILES: [2H]C([2H])([2H])c1cc(Cl)cc2c(=O)n(C[C@@H](O)C[C@H]3NCCC[C@@H]3O)cnc12 ; Cl] | 6-Chloro-3-{(S)- 2-hydroxy-3-[(2R, 3S)-3-hydroxy- piperidin-2-yl]- propyl}-8-(methyl- d3)quinazolin- 4(3H)-one hydrochloride | 355.2 | A:2.55 | (400 MHz, CD₃OD) δ 8.65 (m, 1H), 8.15-8.11 (m, 1H), 7.84-7.79 (m, 1H), 4.39- 4.26 (m, 2H), 4.05-3.98 (m, 1H), 3.62-3.58 (m, 1H), 3.29-3.24 (m, 1H), 3.13- 3.17 (m, 1H), 3.03-2.94 (m, 1H), 2.41-2.35 (m, 1H), 2.13-2.07 (m, 1H), 2.06- |
| | | | | | 1.98 (m, 1H), 1.78-1.55 |
| | | | | | (m, 3H). |
| 36 | 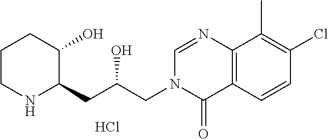 [SMILES: Cc1c(Cl)ccc2c(=O)n(C[C@@H](O)C[C@H]3NCCC[C@@H]3O)cnc12 ; Cl] | 7-Chloro-3-{(S)- 2-hydroxy-3-[(2R, 3S)-3-hydroxy- piperidin-2-yl]- propyl}-8-methyl- quinazolin-4(3H)- one hydrochloride | 352.3 | A:2.55 | (400 MHz, CD₃OD) δ 8.29 (s, 1H), 8.10 (d, J = 8.8 Hz, 1H), 7.58 (d, J = 8.8 Hz, 1H), 4.28-4.22 (m, 2H), 3.98 (dd, J = 14.0, 7.6 Hz, 1H), 3.6-3.48 (m, 1H), 3.30-3.23 (m, 1H), 3.18- 3.09 (m, 1H), 3.06-2.91 (m, 1H), 2.68 (s, 3H), |
| | | | | | 2.37 (dt, J = 15.0, 3.2 Hz, |
| | | | | | 1H), 2.12-1.95 (m, 2H), |
| | | | | | 1.80-1.62 (m, 1H), 1.61- |
| | | | | | 1.51 (m, 2H). |
| 37 | 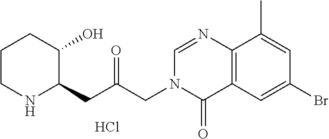 [SMILES: Cc1cc(Br)cc2c(=O)n(CC(=O)C[C@H]3NCCC[C@@H]3O)cnc12 ; Cl] | 6-Bromo-3-{(S)- 2-hydroxy-3-[(2R, 3S)-3-hydroxy- piperidin-2-yl]- propyl}-8-methyl- quinazolin-4(3H)- one hydrochloride | 397.8 | A:2.75 | (400 MHz, CD₃OD) δ 8.62 (d, J = 1.6 Hz, 1H), 8.27 (d, J = 2.0 Hz, 1H), 7.93 (s, 1H), 4.36-4.26 (m, 2H), 4.03-2.97 (m, 1H), 3.60- 3.55 (m, 1H), 3.29-3.30 (m, 1H), 3.18-3.11 (m, 1H), 3.02-2.95 (m, 1H), 2.63 (s, 3H), 2.38 (d, J = 14.8 Hz, |
| | | | | | 1H), 2.12-1.98 (m, 2H), |
| | | | | | 1.75- 1.57 (m, 3H). |
| 38 | 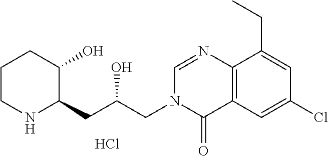 [SMILES: CCc1cc(Cl)cc2c(=O)n(C[C@@H](O)C[C@H]3NCCC[C@@H]3O)cnc12 ; Cl] | 6-Chloro-8-ethyl- 3-{(S)-2-hydroxy- 3-[(2R,3S)-3- hydroxypiper- idin-2-yl]propyl}- quinazolin-4(3H)- one hydrochloride | 366.0 | A: 2.94 | (400 MHz, CD₃OD) δ 8.34 (s, 1H), 8.09 (d, J = 2.4 Hz, 1H), 7.72 (d, J = 2.0 Hz, 1H), 4.31-4.22 (m, 2H), 3.99 (dd, J = 13.2, 8.0 Hz, 1H), 3.61-3.53 (m, 1H), 3.29-3.28 (m, 1H), 3.18-3.12 (m, 1H), 3.06 (q, J = 7.6 Hz, 2H), 3.05- 2.97 (m, 1H), 2.41-2.34 |
| | | | | | (m, 1H), 2.13-2.05 (m, |
| | | | | | 1H), 2.04-1.98 (m, 1H), |
| | | | | | 1.75-1.70 (m, 1H), 1.65- |
| | | | | | 1.56 (m, 2H), 1.31 (t, J = |
| | | | | | 7.2 Hz, 3H). |
| 39 | 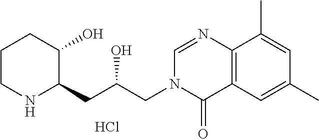 [SMILES: Cc1cc(C)c2ncn(C[C@@H](O)C[C@H]3NCCC[C@@H]3O)c(=O)c2c1 ; Cl] | 3-{(S)-2-Hydroxy- 3-[(2R,3S)-3- hydroxypiperidin- 2-yl]propyl}-6,8- dimethylquin- azolin-4(3H)-one hydrochloride | 332.0 | A:2.30 | ¹H NMR (400 MHz, CD₃OD): δ 9.14 (d, J = 2.0 Hz, 1H), 8.04 (s, 1H), 7.74 (s, 1H), 4.47 (dd, J = 13.2, 2.4 Hz, 1H), 4.32-4.27 (m, 1H), 4.03 (dd, J = 13.6, 8.4 Hz, 1H), 3.62-3.58 (m, 1H), 3.30-3.25 (m, 1H), 3.19-3.15 (m, 1H), 3.02- |
| | | | | | 2.96 (m, 1H), 2.64 (s, 3H), |
| | | | | | 2.52 (s, 3H), 2.40 (d, J = |
| | | | | | 15.2 Hz, 1H), 2.12-2.08 |
| | | | | | (m, 1H), 2.03-1.99 (m, |
| | | | | | 1H), 1.77-1.62 (m, 3H). |
| 40 | 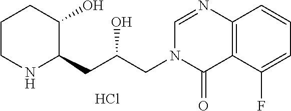 [SMILES: O=c1c2c(F)cccc2ncn1C[C@@H](O)C[C@H]1NCCC[C@@H]1O ; Cl] | 5-Fluoro-3-{(S)- 2-hydroxy-3-[(2R, 3S)-3-hydroxy- piperidin-2-yl]- propyl}quin- azolin-4(3H)-one hydrochloride | 322.2 | A:1.61 | (400 MHz, CD₃OD) δ 8.94 (s, 1H), 7.99-7.94 (m, 1H), 7.59 (d, J = 8.40 Hz, 1H), 7.43 (dd, J = 10.8, 8.4 Hz, 1H), 4.38 (dd, J = 13.5, 3.0 Hz, 1H), 4.32-4.25 (m, 1H), 3.94 (dd, J = 13.5, 8.1, |
| | | | | | Hz, 1H), 3.64-3.57 (m, |
| | | | | | 1H), 3.31-3.25 (m, 1H), |
| | | | | | 3.21-3.13 (m, 1H), 3.05- |
| | | | | | 2.95 (m, 1H), 2.42-2.34 |
| | | | | | (m, 1H), 2.15-2.05 (m, 1H), |
| | | | | | 2.04-1.98 (m, 1H), 1.81- |
| | | | | | 1.53 (m, 3H). |
| 41 | 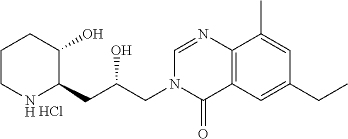 [SMILES: CCc1cc(C)c2ncn(C[C@@H](O)C[C@H]3NCCC[C@@H]3O)c(=O)c2c1 ; Cl] | 6-Ethyl-3-{(S)-2- hydroxy-3-[(2R, 3S)-3-hydroxy- piperidin-2-yl]- propyl}-8-methyl- quinazolin-4(3H)- one hydrochloride | 346.3 | A:2.70 | (400 MHz, CD₃OD) δ 8.98 (s, 1H), 8.05 (s, 1H), 7.75 (s, 1H), 4.46-4.45 (m, 1H), 4.41-4.28 (m, 1H), 4.06- 4.01 (m, 1H), 3.62-3.60 (m, 1H), 3.32-3.30 (m, 1H), 3.17-3.15 (m, 1H), 3.04- 2.95 (m, 1H), 2.84 (q, J = 7.6 Hz, 2H), 2.65 (s, 3H), |
| | | | | | 2.42-2.38 (m, 1H), 2.12- |
| | | | | | 2.08 (m, 1H), 2.00-1.99 |
| | | | | | (m, 1H), 1.76-1.57 (m, |
| | | | | | 3H), 1.33 (t, J = 7.6 Hz, |
| | | | | | 3H). |
| 42 | 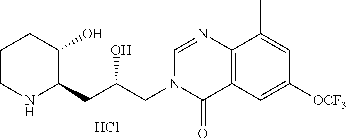 [SMILES: Cc1cc(OC(F)(F)F)cc2c(=O)n(C[C@@H](O)C[C@H]3NCCC[C@@H]3O)cnc12 ; Cl] | 3-{(S)-2-Hydroxy- 3-[(2R,3S)-3- hydroxypiperidin- 2-yl]propyl}-8- methyl-6-(tri- fluoromethoxy)- quinazolin-4(3H)- one hydrochloride | 402.2 | A:3.07 | (400 MHz, CD₃OD) δ 8.32 (s, 1H), 7.96 (s, 1H), 7.64 (s, 1H), 4.32-4.21 (m, 2H), 4.00 (dd, J = 14.0, 7.6 Hz, 1H), 3.60-3.50 (m, 1H), 3.30-3.20 (m, 1H), 3.18- 3.10 (m, 1H), 3.00-2.90 (m, 1H), 2.66 (s, 3H), 2.38 (dt, J = 14.0, 2.8 Hz, 1H), |
| | | | | | 2.16-2.06 (m, 1H), 2.05- |
| | | | | | 1.95 (m, 1H), 1.79-1.69 (m, |
| | | | | | 1H), 1.68-1.51 (m, 2H). |
| 43 | 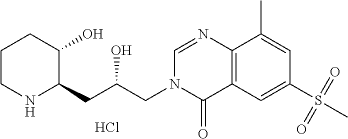 [SMILES: Cc1cc(S(C)(=O)=O)cc2c(=O)n(C[C@@H](O)C[C@H]3NCCC[C@@H]3O)cnc12 ; Cl] | 3-{(S)-2-Hydroxy- 3-[(2R,3S)-3- hydroxypiperidin- 2-yl]propyl}-8- methyl-6-(methyl- sulfonyl)quin- azolin-4(3H)-one hydrochloride | 396.2 | D: 4.61 | (400 MHz, CD₃OD) δ 8.68 (s, 1H), 8.43 (s, 1H), 8.21 (s, 1H), 4.32-4.27 (m, 2H), 4.00 (dd, J = 14.4, 8.4 Hz 1H), 3.62-3.52 (m, 1H), 3.28-3.22 (m, 1H), 3.21 (s, 3H), 3.17-3.14 (m, 1H), 3.01-2.98 (m, 1H), 2.71 (s, 3H), 2.40-2.36 (m, 1H), |
| | | | | | 2.12-2.08 (m, 1H), 2.06- |
| | | | | | 1.98 (m, 1H), 1.80-1.68 |
| | | | | | (m, 1H), 1.67-1.51 (m, 2H). |
| 44 | 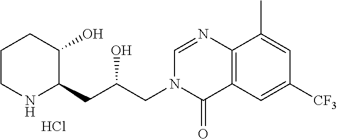 [SMILES: Cc1cc(C(F)(F)F)cc2c(=O)n(C[C@@H](O)C[C@H]3NCCC[C@@H]3O)cnc12 ; Cl] | 3-{(S)-2-Hydroxy- 3-[(2R,3S)-3- hydroxypiper- idin-2-yl]propyl}- 8-methyl-6- trifluoromethyl)- quinazolin-4(3H)- one hydrochloride | 386.1 | D:6.55 | (400 MHz, CD₃OD) δ 8.45 (s, 1H), 8.29 (s, 1H), 7.88 (s, 1H), 4.26-4.13 (m, 2H), 3.92-3.82 (m, 1H), 3.51- 3.41 (m, 1H), 3.25-3.11 (m, 1H), 3.10-2.99 (m, 1H), 2.90-2.82 (m, 1H), 2.58 (s, 3H), 2.31 (dt, J = 14.9, 2.8 Hz, 1H), 2.02-1.82 (m, |
| | | | | | 2H), 1.67-1.40 (m, 3H). |
| 45 | 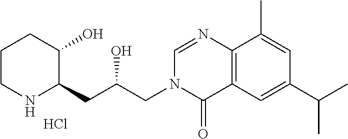 [SMILES: Cc1cc(C(C)C)cc2c(=O)n(C[C@@H](O)C[C@H]3NCCC[C@@H]3O)cnc12 ; Cl] | 3-{(S)-2-Hydroxy- 3-[(2R,3S)-3- hydroxypiperidin- 2-yl]propyl}-6- isopropyl-8- methylquinazolin- 4(3H)-one hydrochloride | 360.3 | A:2.98 | (400 MHz, CD₃OD) δ 8.69 (s, 1H), 8.04 (s, 1H), 7.73 (s, 1H), 4.38-4.27 (m, 2H), 4.03 (dd, J = 13.6, 7.6 Hz, 1H), 3.57 (td, J = 13.3, 3.2 Hz, 1H), 3.29-3.26 (m, 1H), 3.18-3.03 (m, 1H), 3.10 (dd, J = 13.6, 7.2 Hz, 1H), 3.01-2.93 (m, 1H), 2.64 (s, |
| | | | | | 3H), 2.41-2.37 (m, 1H), |
| | | | | | 2.13-2.08 (m, 1H), 2.05- |
| | | | | | 1.98 (m, 1H), 1.74-1.71 |
| | | | | | (m, 1H), 1.67-1.56 (m, 2H), |
| | | | | | 1.34 (d, J = 6.8 Hz, 6H). |
| 46 | 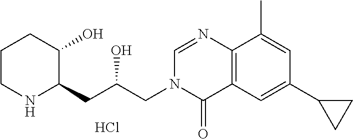 [SMILES: Cc1cc(C2CC2)cc2c(=O)n(C[C@@H](O)C[C@H]3NCCC[C@@H]3O)cnc12 ; Cl] | 6-Cyclopropyl- 3-{(S)-2-hydroxy- 3-[(2R,3S)-3- hydroxypiperidin- 2-yl]propyl}-8- methylquinazolin- 4(3H)-one hydrochloride | 358.2 | C:4.48 | (400 MHz, CD₃OD) δ 8.88 (s, 1H), 7.75 (d, J = 2.0 Hz, 1H), 7.47 (d, J = 0.8 Hz, 1H), 4.33-4.31 (m, 1H), 4.22-4.12 (m, 1H), 3.94-3.88 (m, 1H), 3.48-3.47 (m, 1H), 3.21- 3.20 (m, 1H), 3.17-3.04 (m, 1H), 2.99-2.86 (m, |
| | | | | | 1H), 2.50 (s, 3H), 2.29- |
| | | | | | 2.25 (m, 1H), 2.01-1.98 |
| | | | | | (m, 2H), 1.97-1.88 (m, |
| | | | | | 1H), 1.69-1.40 (m, 3H), |
| | | | | | 1.06-1.01 (m, 2H), 0.76- |
| | | | | | 0.70 (m, 2H). |
| 47 | 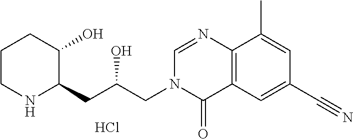 [SMILES: Cc1cc(C#N)cc2c(=O)n(C[C@@H](O)C[C@H]3NCCC[C@@H]3O)cnc12 ; Cl] | 3-{(S)-2-Hydroxy- 3-[(2R,3S)-3- hydroxypiperidin- 2-yl]propyl}-8- methyl-4-oxo- 3,4-dihydroquin- azoline-6- carbonitrile hydro- chloride | 343.0 | E: 5.98 | (400 MHz, DMSO-d₆) δ 9.00-8.94 (br s, 1H), 8.49 (s, 1H), 8.48 (s, 1H), 8.35 (br s, 1H), 8.10 (s, 1H), 4.16-4.02 (m, 2H), 3.90- 3.84 (m, 1H), 3.52-3.46 (m, 1H), 3.14-3.11 (m, 1H), 2.98-2.82 (m, 1H), 2.81-2.72 (m, 1H), 2.56 |
| | | | | | (s, 3H), 2.16-2.12 (m, 1H), |
| | | | | | 1.92-1.88 (m, 1H), 1.79- |
| | | | | | 1.75 (m, 1H), 1.65-1.54 |
| | | | | | (m, 2H), 1.41-1.38 (m, 1H). |
| 48 | 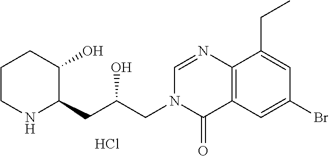 [SMILES: CCc1cc(Br)cc2c(=O)n(C[C@@H](O)C[C@H]3NCCC[C@@H]3O)cnc12 ; Cl] | 6-Bromo-8-ethyl- 3-{(S)-2-hydroxy- 3-[(2R,3S)-3- hydroxypiperidin- 2-yl]propyl}- quinazolin-4(3H)- one hydrochloride | 410.2 | A:3.06 | (400 MHz, CD₃OD) δ 8.67 (d, J = 2.0 Hz, 1H), 8.29 (d, J = 2.4 Hz, 1H), 7.93 (d, J = 2.4 Hz, 1H), 4.34 (dd, J = 13.0, 1.3 Hz, 1H), 4.32-4.28 (m, 1H), 4.01 (d, J = 13.6, 7.6 Hz, 1H), 3.61- 3.56 (m, 1H), 3.30-3.26 (m, 1H), 3.17-3.14 (m, 1H), 3.07 (q, J = 7.6 Hz, |
| | | | | | 2H), 3.01-2.98 (m, 1H), |
| | | | | | 2.41-2.36 (m, 1H), 2.14- |
| | | | | | 2.07 (m, 1H), 2.04-1.98 |
| | | | | | (m, 1H), 1.76-1.57 (m, |
| | | | | | 3H), 1.33 (t, J = 7.6 Hz, |
| | | | | | 3H). |
| 49 | 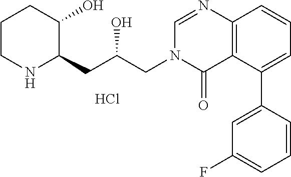 [SMILES: O=c1c2c(-c3cccc(F)c3)cccc2ncn1C[C@@H](O)C[C@H]1NCCC[C@@H]1O ; Cl] | 5-(3-Fluorophenyl- 3-{(S)-2-hydroxy- 3-[(2R,3S)-3- hydroxypiperidin- 2-yl]propyl}quin- azolin-4(3H)-one hydrochloride | 398.2 | C:9.23 | (400 MHz, CD₃OD) δ 9.05 (s, 1H), 8.00 (t, J = 7.6 Hz, 1H), 7.81 (dd, J = 8.2, 1.2 Hz, 1H), 7.53 (dd, J = 7.6, 0.8 Hz, 1H), 7.47-7.38 (m, 1H), 7.17-7.13 (m, 3H), 4.29 (dd, J = 13.6, 2.8 Hz, 1H), 4.19-4.16 (m, 1H), 3.85 (dd, J = 13.6, 8.4 Hz, 1H), 3.57-3.53 (m, 1H), 3.30-3.22 (m, 1H), 3.18- |
| | | | | | 3.08 (m, 1H), 3.02-2.90 |
| | | | | | (m, 1H), 2.32-2.25 (m, |
| | | | | | 1H), 2.11-1.95 (m, 2H), |
| | | | | | 1.80-1.51 (m, 3H). |
| 50 | 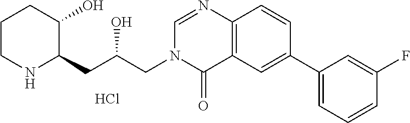 [SMILES: O=c1c2cc(-c3cccc(F)c3)ccc2ncn1C[C@@H](O)C[C@H]1NCCC[C@@H]1O ; Cl] | 6-(3-Fluoro- phenyl)-3-{(S)- 2-hydroxy-3- [(2R,3S)-3- hydroxypiper- idin-2-yl]propyl}- quinazolin-4(3H)- one hydrochloride | 398.3 | C:7.74 | (400 MHz, CD₃OD) δ 9.09 (s, 1H), 8.59 (s, 1H), 8.31 (dd, J = 8.6, 2.4 Hz, 1H), 7.89 (d, J = 8.6 Hz, 1H), 7.62-7.53 (m, 3H), 7.29- 7.16 (m, 1H), 4.46 (dd, J = 13.6, 3.2 Hz, 1H), 4.38-4.27 (m, 1H), 4.02 |
| | | | | | (dd, J = 13.6, 8.4 Hz, 1H), |
| | | | | | 3.68-3.57 (m, 1H), 3.30- |
| | | | | | 3.26 (m, 1H), 3.21-3.15 |
| | | | | | (m, 1H), 3.03-2.92 (m, |
| | | | | | 1H), 2.48-2.37 (m, 1H), |
| | | | | | 2.20-1.92 (m, 2H), 1.82- |
| | | | | | 1.50 (m, 3H). |
| 51 | 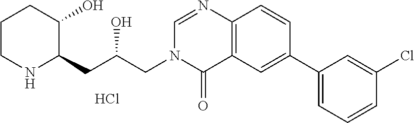 [SMILES: O=c1c2cc(-c3cccc(Cl)c3)ccc2ncn1C[C@@H](O)C[C@H]1NCCC[C@@H]1O ; Cl] | 6-(3-Chloro- phenyl)-3-{(S)- 2-hydroxy-3- [(2R,3S)-3- hydroxypiper- idin-2-yl]propyl}- quinazolin-4(3H)- one hydrochloride | 414.2 | C:5.37 | (400 MHz, CD₃OD) δ 8.67 (s, 1H), 8.43 (d, J = 2.0 Hz, 1H), 8.13 (dd, J = 8.0, 2.0 Hz, 1H), 7.75 (d, J = 8.0 Hz, 1H), 7.67 (s, 1H), 7.60 (d, J = 8.0, 0.8 Hz, 1H), 7.42 (t, J = 7.6 Hz, 1H), 7.38-7.30 (m, 1H), |
| | | | | | 4.28 (dd, J = 13.2, 3.2 Hz, |
| | | | | | 1H), 4.24-4.19 (m, 1H), |
| | | | | | 3.90 (dd, J = 13.4, 8.0 Hz, |
| | | | | | 1H), 3.50-3.42 (m, 1H), |
| | | | | | 3.22-3.17 (m, 1H), 3.09- |
| | | | | | 3.02 (m, 1H), 2.90-2.80 |
| | | | | | (m, 1H), 2.32-2.23 (m, |
| | | | | | 1H), 2.01-1.93 (m, 1H), |
| | | | | | 1.92-1.82 (m, 1H), 1.65- |
| | | | | | 1.40 (m, 3H). |
| 52 | 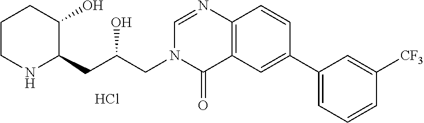 [SMILES: O=c1c2cc(-c3cccc(C(F)(F)F)c3)ccc2ncn1C[C@@H](O)C[C@H]1NCCC[C@@H]1O ; Cl] | 3-{(S)-2- hydroxy-3- [(2R,3S)-3- hydroxypiperidin- 2-yl]propyl}-6- (3-(trifluoro- methyl)phenyl)- quinazolin- | 448.0 | C:7.55 | (400 MHz, CD₃OD) δ 9.08 (s, 1H), 8.62 (d, J = 2.4 Hz, 1H), 8.35 (dd, J = 8.6, 2.0 Hz, 1H), 8.07-8.05 (m, 2H), 7.93 (d, J = 8.8 Hz, 1H), 7.80-7.75 (m, 2H), 4.47 (dd, J = 13.6, 3.2 Hz, 1H), 4.39-4.30 (m, 1H), |
| | | 4(3H)-one | | | 4.03 (dd, J = 13.8, 8.4 Hz, |
| | | hydrochloride | | | 1H), 3.65-3.58 (m, 1H), |
| | | | | | 3.34-3.32 (m, 1H), 3.22- |
| | | | | | 3.16 (m, 1H), 3.03-2.98 |
| | | | | | (m, 1H), 2.45-2.38 (m, |
| | | | | | 1H), 2.15-2.09 (m, 1H), |
| | | | | | 2.08-2.00 (m, 1H), 1.81- |
| | | | | | 1.58 (m, 3H). |
| 53 | 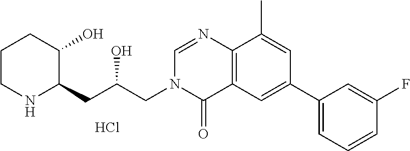 [SMILES: Cc1cc(-c2cccc(F)c2)cc2c(=O)n(C[C@@H](O)C[C@H]3NCCC[C@@H]3O)cnc12 ; Cl] | 6-(3-Fluoro- phenyl)-3-{(S)- 2-hydroxy-3- [(2R,3S)-3- hydroxypiper- idin-2-yl]- propyl}-8-methyl- quinazolin-4(3H)- one hydrochloride | 412.2 | E:7.50 | (400 MHz, CD₃OD) δ 8.31- 8.21 (m, 2H), 7.92 (d, J = 1.2 Hz, 1H), 7.47-7.36 (m, 3H), 7.11-6.99 (m, 1H), 4.28-4.12 (m, 2H), 3.99- 3.83 (m, 1H), 3.5-3.38 (m, 1H), 3.22-3.12 (m, 1H), 3.09-3.00 (m, 1H), 2.95- 2.81 (m, 1H), 2.58 (s, 3H), 2.32-2.20 (m, 1H), 2.04- |
| | | | | | 1.87 (m, 2H), 1.69-1.35 |
| | | | | | (m, 3H). |
| 54 | 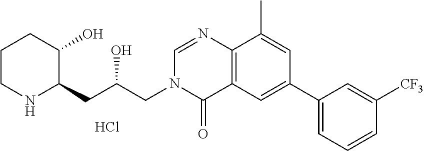 [SMILES: Cc1cc(-c2cccc(C(F)(F)F)c2)cc2c(=O)n(C[C@@H](O)C[C@H]3NCCC[C@@H]3O)cnc12 ; Cl] | 3-{(S)-2-Hydroxy- 3-[(2R,3S)-3- hydroxypiper- idin-2-yl]propyl}- 8-methyl-6-(3- (trifluoromethyl)- phenyl)quin- azolin-4(3H)-one hydrochloride | 462.4 | C:7.23 | (400 MHz, CD₃OD) δ 8.51 (s, 1H), 8.42 (d, J = 2.0 Hz, 1H), 8.11-8.09 (m, 1H), 8.04-8.01 (m, 2H), 7.75-7.72 (m, 2H), 4.37- 4.30 (m, 2H), 4.08-4.02 (m, 1H), 3.58 (ddd, J = 9.0, 4.0 Hz, 1H), 3.31- 3.25 (m, 1H), 3.20-3.10 (m, 1H), 3.02-2.95 (m, |
| | | | | | 1H), 2.72 (s, 3H), 2.40 |
| | | | | | (dt, J = 15.0, 2.8 Hz, 1H), |
| | | | | | 2.15-1.95 (m, 2H), 1.80- |
| | | | | | 1.52 (m, 3H). |
| 55 | 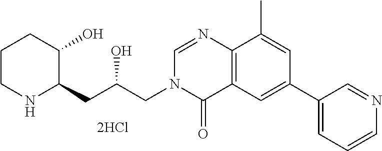 [SMILES: Cc1cc(-c2cccnc2)cc2c(=O)n(C[C@@H](O)C[C@H]3NCCC[C@@H]3O)cnc12 ; "2HCl"] | 3-{(S)-2-Hydroxy- 3-[(2R,3S)-3- hydroxypiper- idin-2-yl]propyl}- 8-methyl-6- (pyridin-3-yl)- quinazolin-4(3H)- one dihydro- chloride | 395.2 | C:5.22 | (400 MHz, CD₃OD) δ 9.36 (s, 1H), 9.09 (d, J = 8.0 Hz, 1H), 8.94 (d, J = 5.6 Hz, 1H), 8.81 (s, 1H), 8.62 (d, J = 2.0 Hz, 1H), 8.29- 8.25 (m, 2H), 4.43 (dd, J = 13.6, 2.8 Hz, 1H), 4.36- 4.31 (m, 1H), 4.04 (dd, J = 13.6, 8.0 Hz, 1H), 3.63- 3.58 (m, 1H), 3.31-3.28 |
| | | | | | (m, 1H), 3.20-3.15 (m, |
| | | | | | 1H), 3.05-2.95 (m, 1H), |
| | | | | | 2.76 (s, 3H), 2.42 (dt, J = |
| | | | | | 15.0, 2.8 Hz, 1H), 2.15- |
| | | | | | 2.08 (m, 1H), 2.07-1.95 |
| | | | | | (m, 1H), 1.88-1.69 (m, |
| | | | | | 2H), 1.62-1.50 (m, 1H). |
| 56 | 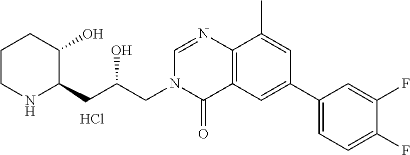 [SMILES: Cc1cc(-c2ccc(F)c(F)c2)cc2c(=O)n(C[C@@H](O)C[C@H]3NCCC[C@@H]3O)cnc12 ; Cl] | 6-(3,4-Difluoro- phenyl)-3-{(S)- 2-hydroxy-3- [(2R,3S)-3- hydroxypiper- idin-2-yl]propyl}- 8-methylquin- azolin-4(3H)-one hydrochloride | 430.2 | C:6.63 | (400 MHz, DMSO-d6) δ 8.86 (br s, 1H), 8.37 (s, 1H), 8.31 (br s, 1H), 8.23 (d, J = 2.0 Hz, 1H), 8.08 (s, 1H), 7.93-7.88 (m, 1H), 7.68-7.63 (m, 1H), 7.61- 7.53 (m, 1H), 4.19-4.11 (m, 2H), 3.94-3.85 (m, 1H), 3.52-3.43 (m, 1H), 3.20-3.11 (m, 1H), 3.03- |
| | | | | | 2.95 (m, 1H), 2.89-2.74 |
| | | | | | (m, 1H), 2.61 (s, 3H), |
| | | | | | 2.20-2.11 (m, 1H), 1.96- |
| | | | | | 1.84 (m, 1H), 1.82-1.73 |
| | | | | | (m, 1H), 1.70-1.49 (m, |
| | | | | | 2H), 1.47-1.35 (m, 1H). |
| 57 | 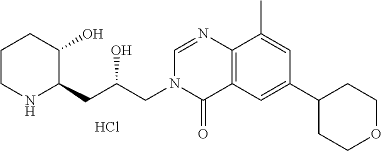 [SMILES: Cc1cc(C2CCOCC2)cc2c(=O)n(C[C@@H](O)C[C@H]3NCCC[C@@H]3O)cnc12 ; Cl] | 3-{(S)-2-Hydroxy- 3-[(2R,3S)-3- hydroxypiperidin- 2-yl]propyl}-8- methyl-6-(tetra- hydro-2H-pyran- 4-yl)quinazolin- (3H)-one hydro- chloride | 402.2 | C: 7.80 | (400 MHz, CD₃OD) 9.17 (s, 1H), 8.10 (d, J = 1.6 Hz, 1H), 7.84 (s, 1H), 4.47 (dd, J = 13.4, 3.2 Hz, 1H), 4.40-4.37 (m, 1H), 4.11-4.07 (m, 3H), 3.65- 3.59 (m, 3H), 3.33-3.29 (m, 1H), 3.21-3.15 (m, 1H), 3.03-2.99 (m, 2H), 2.67 (s, 3H), 2.42-2.39 |
| | | | | | (m, 1H), 2.18-2.09 (m, |
| | | | | | 1H), 2.08-1.99 (m, 1H), |
| | | | | | 1.88-1.83 (m, 4H), 1.78- |
| | | | | | 1.70 (m, 2H), 1.69-1.60 |
| | | | | | (m, 1H). |
| 58 | 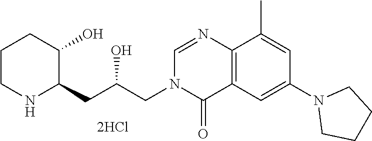 [SMILES: Cc1cc(N2CCCC2)cc2c(=O)n(C[C@@H](O)C[C@H]3NCCC[C@@H]3O)cnc12 ; "2HCl"] | 3-{(S)-2-Hydroxy- 3-[(2R,3S)-3- hydroxypiperidin- 2-yl]propyl}- 8-methyl-6- (pyrrolidin-1-yl)- quinazolin-4(3H)- one dihydro- chloride | 387.2 | E:5.82 | (400 MHz, CD₃OD) δ 8.97 (s, 1H), 7.12 (s, 1H), 7.09 (s, 1H), 4.39 (dd, J = 13.2, 2.0 Hz, 1H), 4.20-4.17 (m, 1H), 3.93 (dd, J = 13.4, 8.8 Hz, 1H), 3.53-3.49 (m, 1H), 3.38-3.25 (m, 4H), 3.18-3.13 (m, 1H), 3.09- 3.05 (m, 1H), 2.89-2.84 |
| | | | | | (m, 1H), 2.52 (s, 3H), |
| | | | | | 2.31-2.27 (m, 1H), 2.08- |
| | | | | | 1.91 (m, 5H), 1.89-1.84 |
| | | | | | (m, 1H), 1.68-1.60 (m, |
| | | | | | 2H), 1.49-1.43 (m, 1H). |
| 59 | 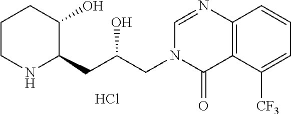 [SMILES: O=c1c2c(C(F)(F)F)cccc2ncn1C[C@@H](O)C[C@H]1NCCC[C@@H]1O ; Cl] | 3-{(S)-2-Hydroxy- 3-[(2R,3S)-3- hydroxypiperidin- 2-yl]propyl}-5- (trifluoromethyl)- quinazolin-4(3H)- one hydrochloride | 372.2 | C:6.08 | (400 MHz, CD₃OD) δ 8.81 (s, 1H), 8.01-7.90 (m, 3H), 4.31-4.26 (m, 1H), 4.21- 4.12 (m, 1H), 3.86-3.82 (m, 1H), 3.53-3.42 (m, 1H), 3.20-3.12 (m, 1H), 3.10-3.01 (m, 1H), 2.90- |
| | | | | | 2.80 (m, 1H), 2.28 (dd, J = |
| | | | | | 15.0, 2.4 Hz, 1H), 2.03- |
| | | | | | 1.80 (m, 2H), 1.70-1.35 |
| | | | | | (m, 3H). |
| 60 | 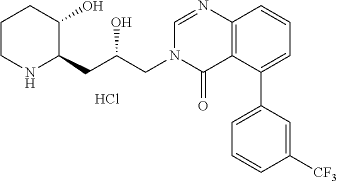 [SMILES: O=c1c2c(-c3cccc(C(F)(F)F)c3)cccc2ncn1C[C@@H](O)C[C@H]1NCCC[C@@H]1O ; Cl] | 3-{(S)-2-Hydroxy- 3-[(2R,3S)-3- hydroxypiperidin- 2-yl]propyl}-5-[3- (trifluoromethyl)- phenyl]quinazolin- 4(3H)-one hydrochloride | 448.3 | C:6.36 | (400 MHz, CD₃OD) δ 8.73 (s, 1H), 7.96 (t, J = 8.4 Hz, 1H), 7.82 (dd, J = 8.4, 1.2 Hz, 1H), 7.71-7.70 (m, 1H), 7.63-7.59 (m, 3H), 7.47 (dd, J = 7.2, 1.2 Hz, 1H), 4.22 (dd, J = 13.6, 2.8 Hz, 1H), 4.20-4.11 (m, 1H), 3.83 (dd, J = 13.6, 8.0 Hz, 1H), 3.56-3.53 (m, 1H), 3.30-3.26 (m, 1H), 3.12- |
| | | | | | 3.08 (m, 1H), 2.98-2.90 |
| | | | | | (m, 1H), 2.30-2.26 (m, |
| | | | | | 1H), 2.10-1.93 (m, 2H), |
| | | | | | 1.78-1.56 (m, 3H). |
| 61 | 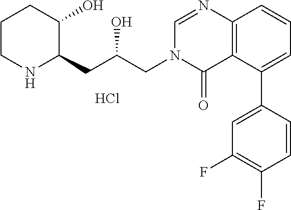 [SMILES: O=c1c2c(-c3ccc(F)c(F)c3)cccc2ncn1C[C@@H](O)C[C@H]1NCCC[C@@H]1O ; Cl] | 5-(3,4-Difluoro- phenyl)-3-{(S)- 2-hydroxy-3- [(2R,3S)-3- hydroxypiper- idin-2-yl]propyl}- quinazolin-4(3H)- one hydrochloride | 416.3 | C:5.23 | (400 MHz, CD₃OD) δ 9.12 (s, 1H), 8.01 (t, J = 8.0 Hz, 1H), 7.82 (d, J = 7.6 Hz, 1H), 7.55 (d, J = 7.2 Hz, 1H), 7.32-7.20 (m, 2H), 7.15 (s, 1H), 4.31 (d, J = 13.6 Hz, 1H), 4.21-4.19 (m, 1H), 3.88-3.82 (m, 1H), 3.60-3.52 (m, 1H), 3.30-3.26 (m, 1H), 3.20- 3.08 (m, 1H), 2.97 (t, J = 11.6 Hz, 1H), 2.30 (d, J = 14.8 Hz, 1H), 2.10-1.98 |
| | | | | | (m, 2H), 1.80-1.52 (m, 3H). |
| 62 | 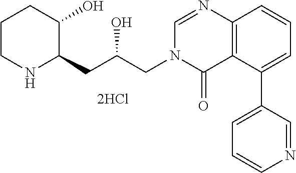 [SMILES: O=c1c2c(-c3cccnc3)cccc2ncn1C[C@@H](O)C[C@H]1NCCC[C@@H]1O ; "2HCl"] | 3-{(S)-2-Hydroxy- 3-[(2R,3S)-3- hydroxypiperidin- 2-yl]propyl}-5- (pyridin-3-yl)- quinazolin-4(3H)- one dihydro- chloride | 381.3 | G: 8.32 | (400 MHz, CD₃OD) δ 9.05 (s, 1H), 9.00-8.91 (m, 2H), 8.72 (d, J = 8.0 Hz, 1H), 8.27-8.15 (m, 1H), 8.14- 8.07 (m, 1H), 8.02-7.96 (m, 1H), 7.72-7.63 (m, 1H), 4.34-4.17 (m, 2H), 3.93-3.70 (m, 1H), 3.64- 3.52 (m, 1H), 3.28-3.23 (m, 1H), 3.20-2.97 (m, 2H), 2.38-2.22 (m, 1H), |
| | | | | | 2.18-2.02 (m, 1H), 2.01- |
| | | | | | 1.98 (m, 1H), 1.81-1.48 |
| | | | | | (m, 3H). |
| 63 | 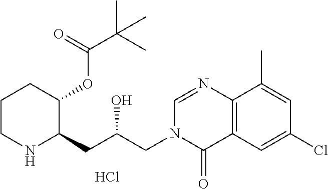 [SMILES: Cc1cc(Cl)cc2c(=O)n(C[C@@H](O)C[C@H]3NCCC[C@@H]3OC(=O)C(C)(C)C)cnc12 ; Cl] | (2R,3S)-2-{(S)- 3-[6-chloro-8- methyl-4-oxoquin- azolin-3(4H)-yl]- 2-hydroxypropyl}- piperidin-3-yl- pivalate hydro- chloride | 436.0 | A:3.877 | (400 MHz, DMSO-d6: D₂O (~20:1 v/v)) δ 8.28 (s, 1H), 7.93 (d, J = 2.4 Hz, 1H), 1.11 (s, 1H), 4.64-4.73 (m, 1H), 4.06-3.95 (m, 2H), 3.93-3.91 (m, 1H), 3.41- 3.40 (m, 1H), 3.21-3.18 (m, 1H), 2.96-2.94 (m, 1H), 2.51 (s, 3H), 1.95- 1.93 (m, 1H), 1.83-1.79 (m, 2H), 1.66-1.64 (m, 1H), 1.60-1.49 (m, 2H), |
| | | | | | 1.095 (s, 9H). |
| 64 | 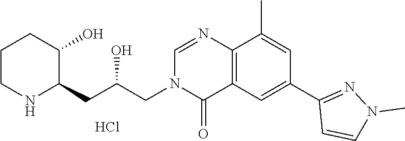 [SMILES: Cc1cc(-c2ccn(C)n2)cc2c(=O)n(C[C@@H](O)C[C@H]3NCCC[C@@H]3O)cnc12 ; Cl] | 3-{(S)-2-hydroxy- 3-[(2R,3S)-3- hydroxypiperidin- 2-yl]propyl}-8- methyl-6-(1- methyl-1H- pyrazol-3-yl)- quinazolin-4(3H)- one hydrochloride | 398.2 | E:5.887 | (400 MHz, DMSO-d₆) δ 9.13 (br s, 1H), 8.44-8.38 (m, 3H), 8.14 (d, J = 1.2 Hz, 1H), 7.79 (d, J = 2.0 Hz, 1H), 6.82 (d, J = 2.0 Hz, 1H), 4.17-4.14 (m, 2H), 3.93-3.88 (m, 4H), 3.54-3.49 (m, 1H), 3.17- 3.11 (m, 1H), 3.01-2.99 |
| | | | | | (m, 1H), 2.81-2.78 (m, |
| | | | | | 1H), 2.60 (s, 3H), 2.18- |
| | | | | | 2.14 (m, 1H), 1.93-1.89 |
| | | | | | (m, 1H), 1.88-1.64 (m, 3H), |
| | | | | | 1.45-1.31 (m, 1H). |
| 65 | 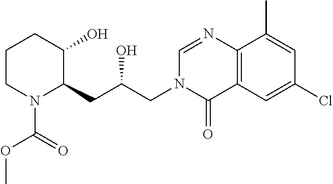 [SMILES: COC(=O)N1CCC[C@H](O)[C@H]1C[C@H](O)Cn1cnc2c(C)cc(Cl)cc2c1=O] | Methyl (2R,3S)-2- ((S)-3-(6-chloro- 8-methyl-4- oxoquinazolin- 3(4H)-yl)-2- hydroxypropyl)- 3-hydroxy- piperidine-1- carboxylate | 410.2 | C: 8.58 | (400 MHz, CD₃OD) δ 8.12 (s, 1H), 7.94 (d, J = 2.40 Hz, 1H), 7.56 (dd, J = 2.40, 0.80 Hz, 1H), 4.47- 4.32 (m, 1H), 4.29-4.19 (m, 1H), 3.99-3.80 (m, 3H), 3.72-3.68 (m, 1H), 3.62 (s, 3H), 2.92-2.81 (m, 1H), 2.49 (s, 3H), 1.85-1.59 (m, 5H), 1.35- 1.28 (m, 1H). |
| 66 | 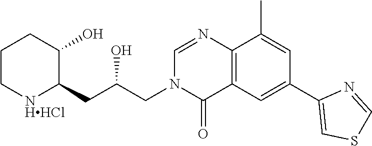 | 3-((S)-2-hydroxy- 3-((2R,3S)-3- hydroxypiperidin- 2-yl)propyl)-8- methyl-6-(thiazol- 4-yl)quinazolin- 4(3H)-one hydro- chloride | 401.2 | C: 7.20 | (400 MHz, CD₃OD): δ 9.19 (d, J = 2.0 Hz, 1H), 8.80 (s, 1H), 8.78 (d, J = 2.5 Hz, 1H), 8.42 (d, J = 2.5 Hz, 1H), 8.19 (d, J = 2.0 Hz, 1H), 4.41 (dd, J = 13.6, 3.0 Hz, 1H), 4.33-4.31 (m, 1H), 4.05 (dd, J = 13.8, 8.0 Hz, 1H), 3.60 (td, J = 9.0, |
| | | | | | 4.0 Hz, 1H), 3.28-3.25 (m, |
| | | | | | 1H), 3.19-3.15 (m, 1H), |
| | | | | | 3.04-2.99 (m, 1H), 2.72 (s, |
| | | | | | 3H), 2.41 (dt, J = 15.1, 3.0 |
| | | | | | Hz, 1H), 2.16-2.09 (m, |
| | | | | | 1H), 2.06-1.96 (m, 1H), |
| | | | | | 1.79-1.54 (m, 3H) |
| 67 | 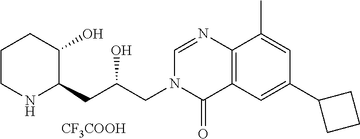 | 6-cyclobutyl-3- {(S)-2-hydroxy- 3-[(2R,3S)-3- hydroxypiperidin- 2-yl]propyl}-8- methylquinazolin- 4(3H)-one | 372.2 | E: 5.09 | (400 MHz, CD₃OD) δ 8.21 (s, 1H), 7.97 (d, J = 2.0 Hz, 1H), 7.60 (s, 1H), 4.32- 4.21 (m, 2H), 4.06-3.98 (m, 1H), 3.75-3.64 (m, 1H), 3.58-3.48 (m, 1H), 3.32- 3.25 (m, 1H), 3.18-3.10 (m, 1H), 3.02-2.92 (m, 1H), 2.60 (s, 3H), 2.48-2.31 |
| | | | | | (m, 3H), 2.29-1.89 (m, |
| | | | | | 6H), 1.78-1.65 (m, 1H), |
| | | | | | 1.64-1.52 (m, 2H). |
| 68 | 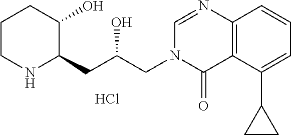 [SMILES: O=c1c2c(C3CC3)cccc2ncn1C[C@@H](O)C[C@H]1NCCC[C@@H]1O ; Cl] | 5-Cyclopropyl-3- {(S)-2-hydroxy- 3-[(2R,3S)-3- hydroxypiperidin- 2-yl]propyl}- quinazolin-4(3H)- one hydrochloride | 344.2 | C:7.98 | (400 MHz, DMSO-d₆) δ 8.77-8.75 (br s, 1H), 8.31 (s, 1H), 8.27-8.24 (br s, 1H), 7.66 (t, J = 8.0 Hz, 1H), 7.45 (dd, J = 8.0, 0.8 Hz, 1H), 7.07 (d, J = 7.2 Hz, 1H), 4.14-4.10 (m, 2H), 3.82-3.77 (m, 1H), 3.56-3.48 (m, 2H), 3.18- |
| | | | | | 3.11 (m, 1H), 3.06-2.92 |
| | | | | | (m, 1H), 2.86-2.75 (m, 1H), |
| | | | | | 2.18-2.12 (m, 1H), 1.95- |
| | | | | | 1.85 (m, 1H), 1.80-1.75 |
| | | | | | (m, 1H), 1.68-1.52 (m, |
| | | | | | 2H), 1.48-1.39 (m, 1H), |
| | | | | | 1.03-1.00 (m, 2H), 0.78- |
| | | | | | 0.76 (m, 2H). |
| 69 | 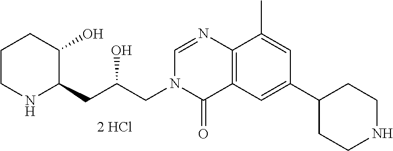 [SMILES: Cc1cc(C2CCNCC2)cc2c(=O)n(C[C@@H](O)C[C@H]3NCCC[C@@H]3O)cnc12 ; "2 HCl"] | 3-{(S)-2-hydroxy- 3-[(2R,3S)-3- hydroxypiperidin- 2-yl]propyl}-8- methyl-6-(piper- idin-4-yl)quin- azolin-4(3H)- one dihydro- chloride | 401.5 | C, 7.70 | (400 MHz, CD₃OD): δ 8.68 (s, 1H), 8.07 (d, J = 2.0 Hz, 1H), 7.75 (d, J = 2.0 Hz, 1H), 4.37 (dd, J = 13.6, 3.2 Hz, 1H), 4.33-4.28 (m, 1H), 4.01 (dd, J = 13.6, 8.0 Hz, 1H), 3.59-3.55 (m, 3H), 3.29-3.12 (m, 5H), 3.03- 2.96 (m, 1H), 2.65 (s, 3H), 2.39 (dt, J = 15.0, 2.8 Hz, |
| | | | | | 1H), 2.22-2.07 (m, 3H), |
| | | | | | 2.05-1.97 (m, 3H), 1.79- |
| | | | | | 1.50 (m, 3H) |
| 70 | 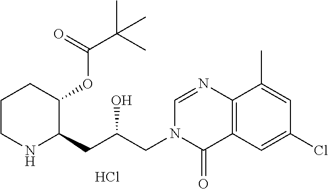 [SMILES: Cc1cc(Cl)cc2c(=O)n(C[C@@H](O)C[C@H]3NCCC[C@@H]3OC(=O)C(C)(C)C)cnc12 ; Cl] | (2R,3S)-2-{(S)-3- [6-chloro-8- methyl-4-oxo- quinazolin-3(4H)- yl]-2-hydroxy- propyl}piperidin- 3-ylpivalate hydro- chloride | 436.0 | A:3.877 | (400 MHz, DMSO-d6: D₂O (~20:1 v/v)) δ 8.28 (s, 1H), 7.93 (d, J = 2.4 Hz, 1H), 7.77 (s, 1H), 4.64-4.73 (m, 1H), 4.06- 3.95 (m, 2H), 3.93-3.91 (m, 1H), 3.41-3.40 (m, 1H), 3.21-3.18 (m, 1H), 2.96-2.94 (m, 1H), 2.51 (s, 3H), 1.95-1.93 (m, 1H), 1.83-1.79 (m, 2H), 1.66- 1.64 (m, 1H), 1.60-1.49 |
| | | | | | (m, 2H), 1.095 (s, 9H). |
| 71 | 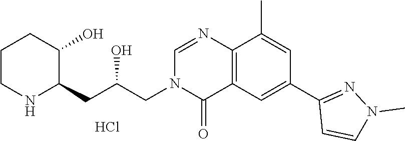 [SMILES: Cc1cc(-c2ccn(C)n2)cc2c(=O)n(C[C@@H](O)C[C@H]3NCCC[C@@H]3O)cnc12 ; Cl] | 3-{(S)-2-hydroxy- 3-[(2R,3S)-3- hydroxypiperidin- 2-yl]propyl}-8- methyl-6-(1- methyl-1H- pyrazol-3-yl)- quinazolin-4(3H)- one hydrochloride | 398.2 | E:5.887 | (400 MHz, DMSO-d₆,) δ 9.13 (br s, 1H), 8.44-8.38 (m, 3H), 8.14 (d, J = 1.2 Hz, 1H), 7.79 (d, J = 2.0 Hz, 1H), 6.82 (d, J = 2.0 Hz, 1H), 4.17-4.14 (m, 2H), 3.93-3.88 (m, 4H), 3.54-3.49 (m, 1H), 3.17- 3.11 (m, 1H), 3.01-2.99 |
| | | | | | (m, 1H), 2.81-2.78 (m, |
| | | | | | 1H), 2.60 (s, 3H), 2.18- |
| | | | | | 2.14 (m, 1H), 1.93-1.89 |
| | | | | | (m, 1H), 1.88-1.64 (m, |
| | | | | | 3H), 1.45-1.31 (m, 1H). |

### Example 72: 6-Chloro-3-{[(3S,4aR,5S)-5-hydroxyhexahydro-1H,3H-pyrido[1,2-c][1,3]oxazin-3-yl]methyl}-8-methylquinazolin-4(3H)-one

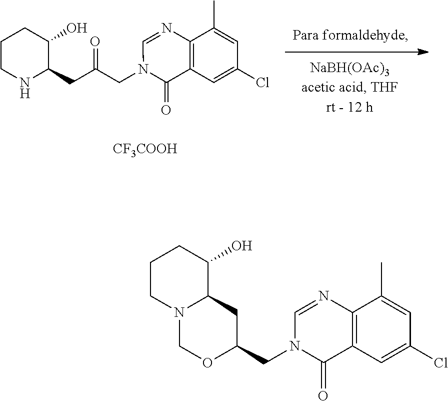

To a stirred solution of 6-chloro-3-{3-[(2R,3S)-3-hydroxypiperidin-2-yl]-2-oxopropyl}-8-methylquinazolin-4(3H)-one-2,2,2-trifluoroacetic acid (50 mg, 0.107 mmol), para formaldehyde (10.6 mg, 0.118 mmol), and acetic acid (0.1 ml) in THE (10 mL), sodium triacetoxyborohydride (45 mg, 0.215 mmol) was added to the above stirred solution at 0° C., was heated at rt and stirred for 12 h. The reaction mixture was quenched with ice, extracted with ethyl acetate (50 mL×2), washed with water (5 mL), brine solution (5 mL), dried over Na₂SO₄ and concentrated in vacuo. The mixture was purified by flash column chromatography (95% EtOAc in petether as an eluent) to afford the 6-chloro-3-{[(3S,4aR,5S)-5-hydroxyhexahydro-1H,3H-pyrido[1,2-c][1,3]oxazin-3-yl]methyl}-8-methylquinazolin-4(3H)-one (25 mg, 63%) as a pale brown solid. LCMS (ESI) m/z: 364.0 [M+H]⁺; (400 MHz, CD₃OD) δ 8.25 (s, 1H), 8.05 (d, J=2.0 Hz, 1H), 7.69 (d, J=2.0 Hz, 1H), 4.43-4.32 (m, 2H), 4.04 (dd, J=14.0, 8.0 Hz, 1H), 3.92-3.82 (m, 1H), 3.63 (d, J=8.0 Hz, 1H), 3.30-3.20 (m, 1H), 2.70-2.61 (m, 1H), 2.60 (s, 3H), 2.21-2.12 (m, 1H), 2.08-1.90 (m, 3H), 1.81-1.71 (m, 1H), 1.70-1.53 (m, 1H), 1.42-1.25 (m, 3H). Analytical HPLC RT=2.71 min (Method A).

Assay for Inhibitors of Prolyl tRNA Synthetase Activity

In order to establish the activities of the compounds highlighted above, we determined extent of inhibition of prolyl tRNA synthetase activity in dose response curves to obtain IC50 values.

The prolyl tRNA synthetase moiety (ProRS) in the cDNA of glutamyl prolyl tRNA synthetase was subcloned, tagged with a single biotin, purified and used in the assay.

Briefly, human ProRS with a 6×N-terminal His tag in pET28N with TVMV BioP was transformed in E. coli BL21 (DE3) cells, selected, overexpressed and purified using a Ni Seph 6 FF XK 16/20 column in the first stage. Ni-NTA eluates were pooled and treated with TVMV protease, and biotinylated using d-Biotin, MgCl₂, ATP and the BirA enzyme. This was rerun through a Ni column as before. The eluates were further enriched and purified using membrane ultrafiltration and a SuperDex 200 XK 16/60 column, and peak fractions were confirmed by SDS-PAGE prior to protein amount determination, aliquoting and storage at −80° C.
ProRS assay was performed in 96 well plates in a final reaction volume of 20 μl, using staggered additions of Tris-Cl (pH 7.5, 60 mM), dithiothreitol (2 mM), ATP (5 mM, pH 7.0), MgCl₂ 6 mM), ProRS (20 nM, with pre-determined active fraction range) and bovine tRNA (20 μg). Controls and compounds, at various concentrations (10 μM to 0.5 nM), were incubated on ice with enzyme mix for 15 min followed by addition of proline (non-labeled and labeled, 3.96 and 1.04 μM (1 μCi) respectively) to initiate the reaction. Reaction was carried out for 10 min at 37° C. and terminated by addition of TCA (10%, 100 μl). Reaction mix was collected on a Whatman GF/B filter using a vacuum filtration manifold. This filter was washed 3 times with 5% TCA followed by 2 washes with ethanol, air dried, and read using a liquid scintillation counter (TopCount NXT) using MicroScint O. IC₅₀ values were calculated and analyzed using ToolSet (4-parameter plot) and depicted herein.

### GSH Trapping Assay

Rat hepatocytes were isolated from male Sprague-Dawley rats (Vivo Biotech Ltd, HYD, India), weighing 230 to 280 g by the two step collagenase perfusion method (seglen, 1976; Berry M N and Friend D S.1969). Rat hepatocytes were cultured in Dulbecco's Modified Eagle's Medium (DMEM, Gibco®, USA) supplemented with 10⁻⁶ M dexamethasone (Gibco®), 5% fetal bovine serum (Gibco®) and cocktail-A mix (CM3000, Gibco®) containing HEPES buffer (PH 7.4), GlutaMAX™, human recombinant insulin, penicillin and streptomycin. Initial cell viability assessed by Trypanblue stain 0.4% (Sigma, UK) and viability more than 80% were considered for culturing. Hepatocytes were seeded in 96 well type-I collagen coated plates (Gibco®) at 4×10⁴ cells/well in 100 μL of the culture medium and incubated at 37° C. in 5% CO₂ and 95% relative humidity (RH) for 4 hr of cell attachment.

After 4 hr incubation, rat hepatocytes were washed twice with pre-warmed (37° C.) DMEM incubation medium supplemented with 10⁻⁷ M dexamethasone and cell maintenance cocktail-B (CM4000, Gibco®) solution to remove unattached cells. Post washing hepatocytes were treated with test and reference compounds in incubation medium at single concentration (30 μM) in duplicate and incubate further for 24 hr at 37° C. with 5% CO₂ and 95% RH. The final concentration of DMSO in medium was 0.5%.

Post 24 hr incubation, hepatocyte samples were quenched with equal volumes of acetonitrile containing 0.1% formic acid followed by vortex mix, sonicate and centrifuge at 12000 rpm at room temperature for 5 min. Supernatant samples were analyzed for GSH adducts on a C18 reverse-phase column (Xbridge™ Shield, C₁₈, 4.6×150 mm, 3.5 μm) with solvent A (10 mM ammonium formate and 0.1% formic acid/water) and solvent B (0.1% formic acid/acetonitrile).

The column was eluted with 5% B for 5 min and then with a linear gradient to 20% B over 10 min and it was maintained up to 15 min, then it was increased linearly to 40% B up to 27 min and then increase to 90% B up to 29 min, and then equilibrate back the column to 5% B up to 33 min at a flow rate of 0.6 ml/min. The column effluent was directed into the ESI source of a LTQ Velos Orbitrap mass spectrometer (Thermo Fisher Scientific, San Jose, Calif.). The ESI conditions were as follows: sheath gas flow rate, 40 arbitrary units; auxiliary gas, 30 arbitrary units; spray voltage, 4.5 kV; capillary temperature, 230° C.; capillary voltage, 30 V; and tube lens offset, 25 V. Data were acquired in positive ion mode using Xcalibur software (Thermo Fisher Scientific) with one full scan followed by two data-dependent scans of the most intense and the second most intense ions. During the data interpretation the characteristic fragments of neutral loss of glycine (75.0320 Da), pyroglutamic acid (129.0426 Da) and loss of neutral moiety GSH (307.0838 Da) and/or a specific fragment of (GSH+H)⁺ (m/z 308.0911) were monitored. Finally the fragments ions with m/z e.g. 179.0485, 177.0328 and 162.0219 Da, which were derived from the glutathione moiety were used to report GSH positive results. The results of the biological testing are shown in Table 4.

TABLE 4

| | | EPRS | GSH |
|---|---|---|---|
| | | IC₅₀ | adduct |
| Ex. | Structure | (nM) | formation |
| 1 | 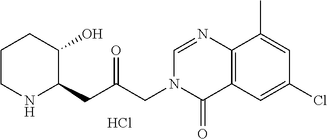 [SMILES: Cc1cc(Cl)cc2c(=O)n(CC(=O)C[C@H]3NCCC[C@@H]3O)cnc12 ; Cl] | 73 | |
| 2 | 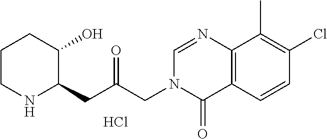 [SMILES: Cc1c(Cl)ccc2c(=O)n(CC(=O)C[C@H]3NCCC[C@@H]3O)cnc12 ; Cl] | 15 | |
| 3 | 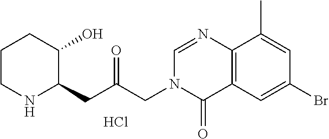 [SMILES: Cc1cc(Br)cc2c(=O)n(CC(=O)C[C@H]3NCCC[C@@H]3O)cnc12 ; Cl] | 4 | Negative |
| 4 | 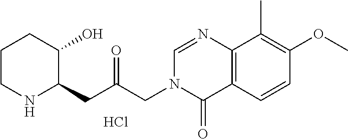 [SMILES: COc1ccc2c(=O)n(CC(=O)C[C@H]3NCCC[C@@H]3O)cnc2c1C ; Cl] | 49 | |
| 5 | 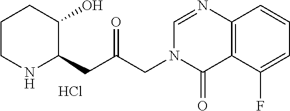 [SMILES: O=C(C[C@H]1NCCC[C@@H]1O)Cn1cnc2cccc(F)c2c1=O ; Cl] | 8 | |
| 6 | 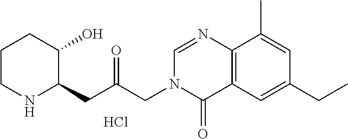 [SMILES: CCc1cc(C)c2ncn(CC(=O)C[C@H]3NCCC[C@@H]3O)c(=O)c2c1 ; Cl] | 3 | |
| 7 | 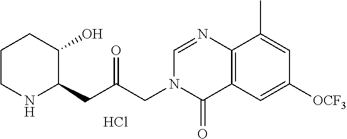 [SMILES: Cc1cc(OC(F)(F)F)cc2c(=O)n(CC(=O)C[C@H]3NCCC[C@@H]3O)cnc12 ; Cl] | 9 | |
| 8 | 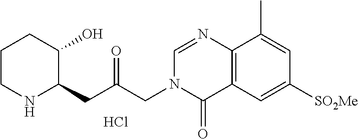 [SMILES: Cc1cc(S(C)(=O)=O)cc2c(=O)n(CC(=O)C[C@H]3NCCC[C@@H]3O)cnc12 ; Cl] | 9 | |
| 9 | 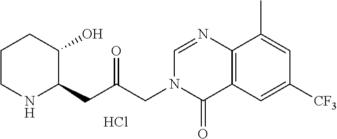 [SMILES: Cc1cc(C(F)(F)F)cc2c(=O)n(CC(=O)C[C@H]3NCCC[C@@H]3O)cnc12 ; Cl] | 7 | |
| 10 | 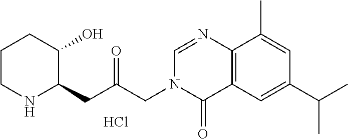 [SMILES: Cc1cc(C(C)C)cc2c(=O)n(CC(=O)C[C@H]3NCCC[C@@H]3O)cnc12 ; Cl] | 6 | |
| 11 | 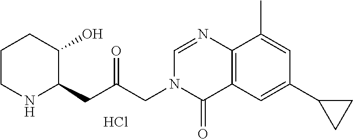 [SMILES: Cc1cc(C2CC2)cc2c(=O)n(CC(=O)C[C@H]3NCCC[C@@H]3O)cnc12 ; Cl] | 6 | |
| 12 | 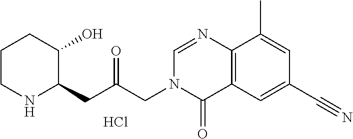 [SMILES: Cc1cc(C#N)cc2c(=O)n(CC(=O)C[C@H]3NCCC[C@@H]3O)cnc12 ; Cl] | 17 | |
| 13 | 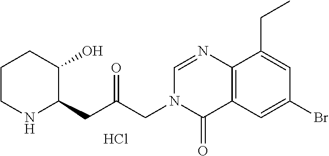 [SMILES: CCc1cc(Br)cc2c(=O)n(CC(=O)C[C@H]3NCCC[C@@H]3O)cnc12 ; Cl] | 6 | |
| 14 | 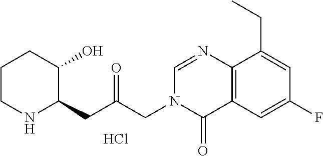 [SMILES: CCc1cc(F)cc2c(=O)n(CC(=O)C[C@H]3NCCC[C@@H]3O)cnc12 ; Cl] | 38 | |
| 15 | 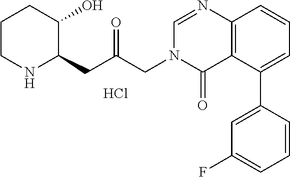 [SMILES: O=C(C[C@H]1NCCC[C@@H]1O)Cn1cnc2cccc(-c3cccc(F)c3)c2c1=O ; Cl] | 6 | |
| 16 | 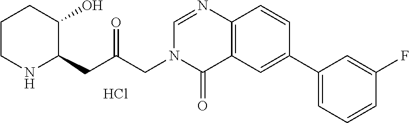 [SMILES: O=C(C[C@H]1NCCC[C@@H]1O)Cn1cnc2ccc(-c3cccc(F)c3)cc2c1=O ; Cl] | 10 | |
| 17 | 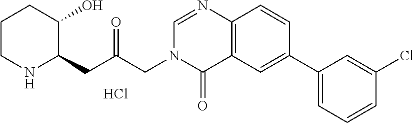 [SMILES: O=C(C[C@H]1NCCC[C@@H]1O)Cn1cnc2ccc(-c3cccc(Cl)c3)cc2c1=O ; Cl] | 28 | |
| 18 | 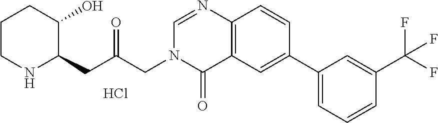 [SMILES: O=C(C[C@H]1NCCC[C@@H]1O)Cn1cnc2ccc(-c3cccc(C(F)(F)F)c3)cc2c1=O ; Cl] | 40 | |
| 19 | 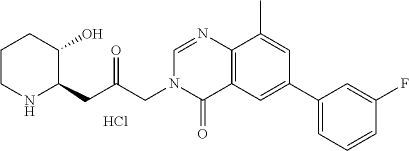 [SMILES: Cc1cc(-c2cccc(F)c2)cc2c(=O)n(CC(=O)C[C@H]3NCCC[C@@H]3O)cnc12 ; Cl] | 9 | |
| 20 | 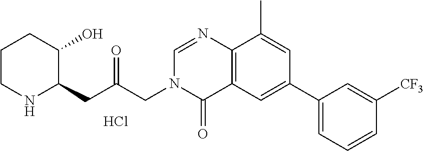 [SMILES: Cc1cc(-c2cccc(C(F)(F)F)c2)cc2c(=O)n(CC(=O)C[C@H]3NCCC[C@@H]3O)cnc12 ; Cl] | 28 | |
| 21 | 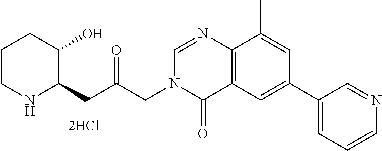 [SMILES: Cc1cc(-c2cccnc2)cc2c(=O)n(CC(=O)C[C@H]3NCCC[C@@H]3O)cnc12 ; "2HCl"] | 8 | |
| 22 | 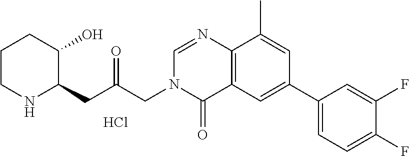 [SMILES: Cc1cc(-c2ccc(F)c(F)c2)cc2c(=O)n(CC(=O)C[C@H]3NCCC[C@@H]3O)cnc12 ; Cl] | 15 | |
| 23 | 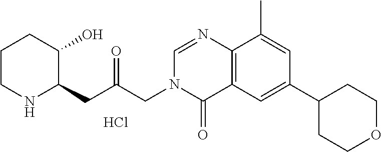 [SMILES: Cc1cc(C2CCOCC2)cc2c(=O)n(CC(=O)C[C@H]3NCCC[C@@H]3O)cnc12 ; Cl] | 12 | |
| 24 | 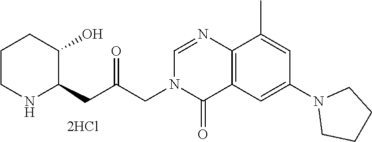 [SMILES: Cc1cc(N2CCCC2)cc2c(=O)n(CC(=O)C[C@H]3NCCC[C@@H]3O)cnc12 ; "2HCl"] | 14 | |
| 25 | 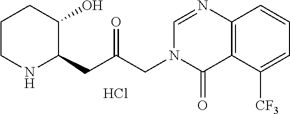 [SMILES: O=C(C[C@H]1NCCC[C@@H]1O)Cn1cnc2cccc(C(F)(F)F)c2c1=O ; Cl] | 10 | |
| 26 | 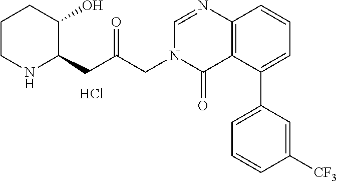 [SMILES: O=C(C[C@H]1NCCC[C@@H]1O)Cn1cnc2cccc(-c3cccc(C(F)(F)F)c3)c2c1=O ; Cl] | 13 | |
| 27 | 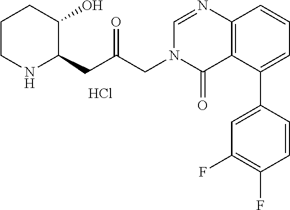 [SMILES: O=C(C[C@H]1NCCC[C@@H]1O)Cn1cnc2cccc(-c3ccc(F)c(F)c3)c2c1=O ; Cl] | 6 | |
| 28 | 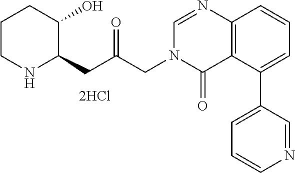 [SMILES: O=C(C[C@H]1NCCC[C@@H]1O)Cn1cnc2cccc(-c3cccnc3)c2c1=O ; "2HCl"] | 6 | |
| 29 | 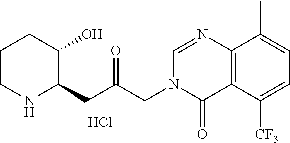 [SMILES: Cc1ccc(C(F)(F)F)c2c(=O)n(CC(=O)C[C@H]3NCCC[C@@H]3O)cnc12 ; Cl] | 313 | |
| 30 | 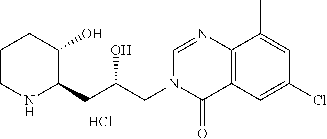 [SMILES: Cc1cc(Cl)cc2c(=O)n(C[C@@H](O)C[C@H]3NCCC[C@@H]3O)cnc12 ; Cl] | 19 | Negative |
| 31 | 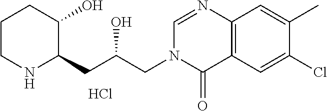 [SMILES: Cc1cc2ncn(C[C@@H](O)C[C@H]3NCCC[C@@H]3O)c(=O)c2cc1Cl ; Cl] | 17 | Negative (detected by mass only) |
| 32 | 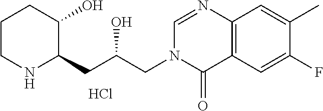 [SMILES: Cc1cc2ncn(C[C@@H](O)C[C@H]3NCCC[C@@H]3O)c(=O)c2cc1F ; Cl] | 55 | Negative |
| 33 | 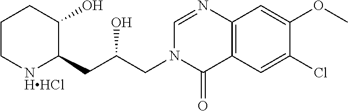 | 116 | Negative |
| 34 | 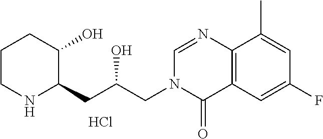 [SMILES: Cc1cc(F)cc2c(=O)n(C[C@@H](O)C[C@H]3NCCC[C@@H]3O)cnc12 ; Cl] | 183 | |
| 35 | 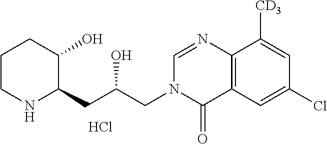 [SMILES: [2H]C([2H])([2H])c1cc(Cl)cc2c(=O)n(C[C@@H](O)C[C@H]3NCCC[C@@H]3O)cnc12 ; Cl] | 32 | |
| 36 | 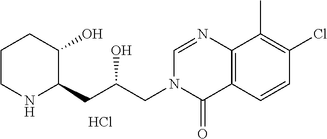 [SMILES: Cc1c(Cl)ccc2c(=O)n(C[C@@H](O)C[C@H]3NCCC[C@@H]3O)cnc12 ; Cl] | 429 | |
| 37 | 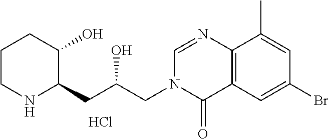 [SMILES: Cc1cc(Br)cc2c(=O)n(C[C@@H](O)C[C@H]3NCCC[C@@H]3O)cnc12 ; Cl] | 62 | |
| 38 | 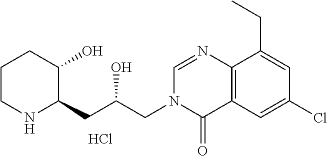 [SMILES: CCc1cc(Cl)cc2c(=O)n(C[C@@H](O)C[C@H]3NCCC[C@@H]3O)cnc12 ; Cl] | 506 | |
| 39 | 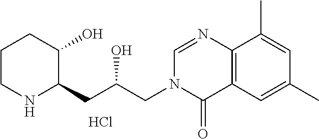 [SMILES: Cc1cc(C)c2ncn(C[C@@H](O)C[C@H]3NCCC[C@@H]3O)c(=O)c2c1 ; Cl] | 158 | |
| 40 | 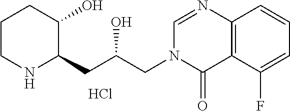 [SMILES: O=c1c2c(F)cccc2ncn1C[C@@H](O)C[C@H]1NCCC[C@@H]1O ; Cl] | 302 | Negative |
| 41 | 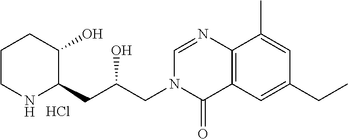 [SMILES: CCc1cc(C)c2ncn(C[C@@H](O)C[C@H]3NCCC[C@@H]3O)c(=O)c2c1 ; Cl] | 23 | |
| 42 | 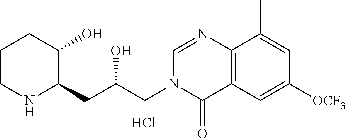 [SMILES: Cc1cc(OC(F)(F)F)cc2c(=O)n(C[C@@H](O)C[C@H]3NCCC[C@@H]3O)cnc12 ; Cl] | 20 | |
| 43 | 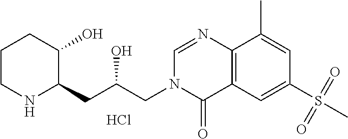 [SMILES: Cc1cc(S(C)(=O)=O)cc2c(=O)n(C[C@@H](O)C[C@H]3NCCC[C@@H]3O)cnc12 ; Cl] | 38 | |
| 44 | 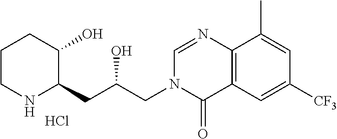 [SMILES: Cc1cc(C(F)(F)F)cc2c(=O)n(C[C@@H](O)C[C@H]3NCCC[C@@H]3O)cnc12 ; Cl] | 20 | |
| 45 | 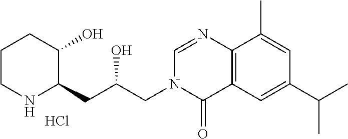 [SMILES: Cc1cc(C(C)C)cc2c(=O)n(C[C@@H](O)C[C@H]3NCCC[C@@H]3O)cnc12 ; Cl] | 21 | |
| 46 | 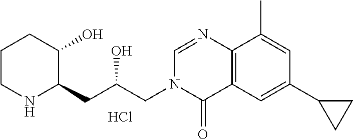 [SMILES: Cc1cc(C2CC2)cc2c(=O)n(C[C@@H](O)C[C@H]3NCCC[C@@H]3O)cnc12 ; Cl] | 34 | |
| 47 | 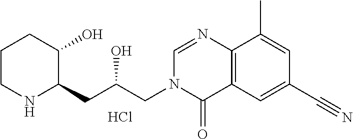 [SMILES: Cc1cc(C#N)cc2c(=O)n(C[C@@H](O)C[C@H]3NCCC[C@@H]3O)cnc12 ; Cl] | 235 | |
| 48 | 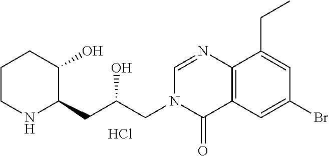 [SMILES: CCc1cc(Br)cc2c(=O)n(C[C@@H](O)C[C@H]3NCCC[C@@H]3O)cnc12 ; Cl] | 83 | Negative |
| 49 | 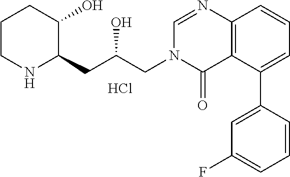 [SMILES: O=c1c2c(-c3cccc(F)c3)cccc2ncn1C[C@@H](O)C[C@H]1NCCC[C@@H]1O ; Cl] | 41 | Negative |
| 50 | 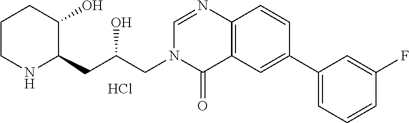 [SMILES: O=c1c2cc(-c3cccc(F)c3)ccc2ncn1C[C@@H](O)C[C@H]1NCCC[C@@H]1O ; Cl] | 14 | |
| 51 | 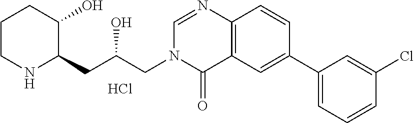 [SMILES: O=c1c2cc(-c3cccc(Cl)c3)ccc2ncn1C[C@@H](O)C[C@H]1NCCC[C@@H]1O ; Cl] | 23 | |
| 52 | 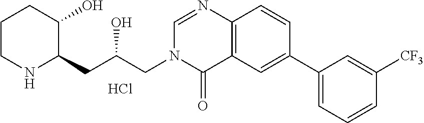 [SMILES: O=c1c2cc(-c3cccc(C(F)(F)F)c3)ccc2ncn1C[C@@H](O)C[C@H]1NCCC[C@@H]1O ; Cl] | 40 | |
| 53 | 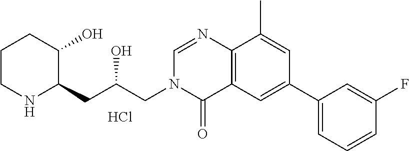 [SMILES: Cc1cc(-c2cccc(F)c2)cc2c(=O)n(C[C@@H](O)C[C@H]3NCCC[C@@H]3O)cnc12 ; Cl] | 9 | |
| 54 | 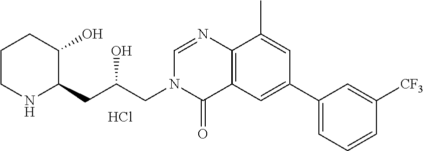 [SMILES: Cc1cc(-c2cccc(C(F)(F)F)c2)cc2c(=O)n(C[C@@H](O)C[C@H]3NCCC[C@@H]3O)cnc12 ; Cl] | 31 | |
| 55 | 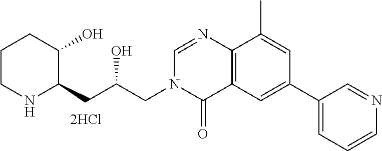 [SMILES: Cc1cc(-c2cccnc2)cc2c(=O)n(C[C@@H](O)C[C@H]3NCCC[C@@H]3O)cnc12 ; "2HCl"] | 11 | |
| 56 | 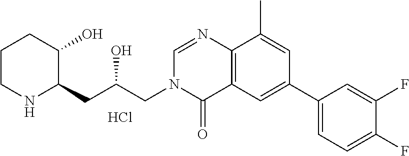 [SMILES: Cc1cc(-c2ccc(F)c(F)c2)cc2c(=O)n(C[C@@H](O)C[C@H]3NCCC[C@@H]3O)cnc12 ; Cl] | 4 | |
| 57 | 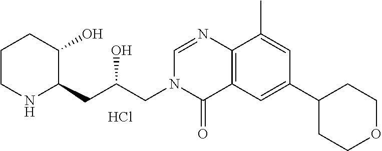 [SMILES: Cc1cc(C2CCOCC2)cc2c(=O)n(C[C@@H](O)C[C@H]3NCCC[C@@H]3O)cnc12 ; Cl] | 52 | |
| 58 | 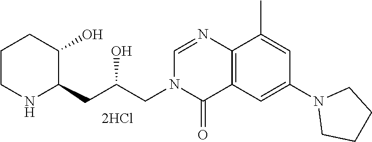 [SMILES: Cc1cc(N2CCCC2)cc2c(=O)n(C[C@@H](O)C[C@H]3NCCC[C@@H]3O)cnc12 ; "2HCl"] | 321 | |
| 59 | 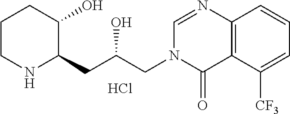 [SMILES: O=c1c2c(C(F)(F)F)cccc2ncn1C[C@@H](O)C[C@H]1NCCC[C@@H]1O ; Cl] | 316 | |
| 60 | 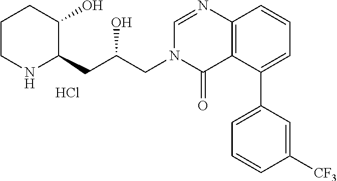 [SMILES: O=c1c2c(-c3cccc(C(F)(F)F)c3)cccc2ncn1C[C@@H](O)C[C@H]1NCCC[C@@H]1O ; Cl] | 38 | |
| 61 | 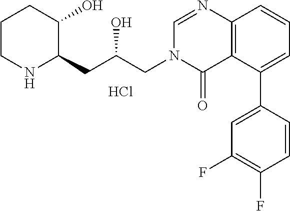 [SMILES: O=c1c2c(-c3ccc(F)c(F)c3)cccc2ncn1C[C@@H](O)C[C@H]1NCCC[C@@H]1O ; Cl] | 69 | |
| 62 | 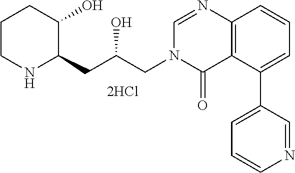 [SMILES: O=c1c2c(-c3cccnc3)cccc2ncn1C[C@@H](O)C[C@H]1NCCC[C@@H]1O ; "2HCl"] | 53 | |
| 63 | 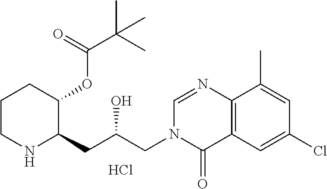 [SMILES: Cc1cc(Cl)cc2c(=O)n(C[C@@H](O)C[C@H]3NCCC[C@@H]3OC(=O)C(C)(C)C)cnc12 ; Cl] | >10000 | |
| 64 | 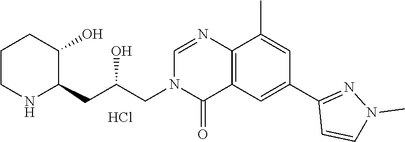 [SMILES: Cc1cc(-c2ccn(C)n2)cc2c(=O)n(C[C@@H](O)C[C@H]3NCCC[C@@H]3O)cnc12 ; Cl] | 30 | |
| 65 | 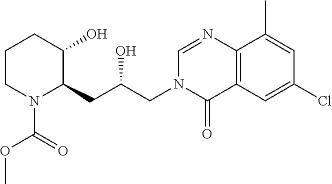 [SMILES: COC(=O)N1CCC[C@H](O)[C@H]1C[C@H](O)Cn1cnc2c(C)cc(Cl)cc2c1=O] | >10000 | |
| 66 | 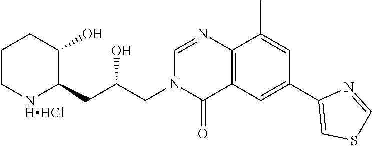 | 13 | |
| 67 | 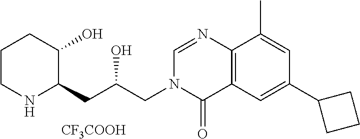 | 18 | |
| 68 | 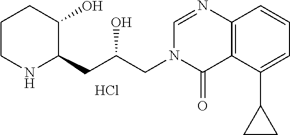 [SMILES: O=c1c2c(C3CC3)cccc2ncn1C[C@@H](O)C[C@H]1NCCC[C@@H]1O ; Cl] | 9 | |
| 69 | 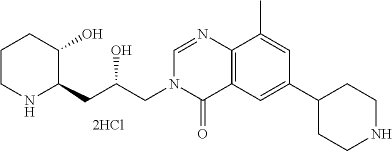 [SMILES: Cc1cc(C2CCNCC2)cc2c(=O)n(C[C@@H](O)C[C@H]3NCCC[C@@H]3O)cnc12 ; "2HCl"] | 106 | |
| 70 | 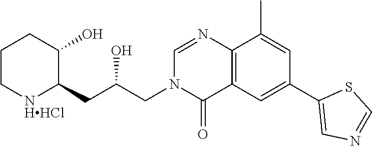 | 20 | |
| 71 | 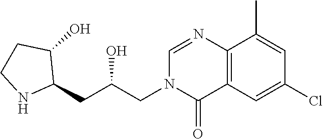 [SMILES: Cc1cc(Cl)cc2c(=O)n(C[C@@H](O)C[C@H]3NCC[C@@H]3O)cnc12] | 4202 | |
| 72 | 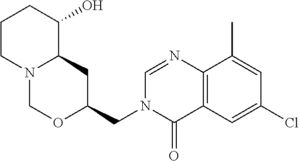 [SMILES: Cc1cc(Cl)cc2c(=O)n(C[C@@H]3C[C@@H]4[C@@H](O)CCCN4CO3)cnc12] | >10000 | |

Throughout this application, various publications are referenced by author name and date, or by patent number or patent publication number. The disclosures of these publications are hereby incorporated in their entireties by reference into this application in order to more fully describe the state of the art as known to those skilled therein as of the date of the invention described and claimed herein. However, the citation of a reference herein should not be construed as an acknowledgement that such reference is prior art to the present invention.

Those skilled in the art will recognize, or be able to ascertain using no more than routine experimentation, numerous equivalents to the specific procedures described herein. Such equivalents are considered to be within the scope of this invention and are covered by the following claims. For example, it is intended in accordance with the present invention that the compounds of the invention can be employed to treat diseases other than the specific diseases disclosed in the description herein. Further, compounds other than those disclosed in the description and Examples herein can be employed. Furthermore, it is intended that specific items within lists of items, or subset groups of items within larger groups of items, can be combined with other specific items, subset groups of items or larger groups of items whether or not there is a specific disclosure herein identifying such a combination.

It will be evident to one skilled in the art that the present disclosure is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

We claim:

1. A compound of Formula I 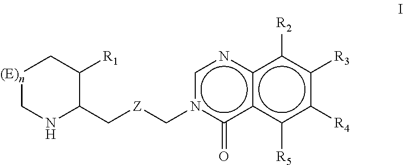
where:
E is CH₂, where n is 0 or 1;
Z is C═O or CH—OH;
R₁ is —OH or —OCOC(CH₃)₃;
R₂ is hydrogen, —CH₃ or —CH₂CH₃;
R₃ is hydrogen, —CH₃, or —OCH₃;
R₄ is selected from hydrogen, halogen, —CH₂CH₃, —CHCH₃CH₃, —OCF₃, —C≡N, substituted or unsubstituted aryl, substituted or unsubstituted heteroaryl, cycloalkyl, 5 or 6 membered substituted or unsubstituted saturated or unsaturated heterocycle, —SO₂CH₃, or —CF₃;
R₅ is selected from hydrogen, halogen, —CF₃, substituted or unsubstituted aryl, or substituted or unsubstituted heteroaryl, or cycloalkyl;
or a pharmaceutically acceptable salt thereof.

2. A compound of claim 1 where R₁ is —OH.

3. A compound of claim 1 where R₂ is —CH₃.

4. A compound of claim 3 where R₃ is hydrogen.

5. A compound of claim 1 where R₂ is hydrogen.

6. A compound of claim 5 where R₃ is —CH₃.

7. A compound of claim 1 where R₂, R₃ and R₄ are hydrogen.

8. A compound of claim 1 where when n=0, Z is ═O, and R₂ and R₅ are H, then R₁ is not —OH.

9. A compound of claim 1 where R₄ is a substituted or unsubstituted aryl.

10. A compound of claim 9 where R₄ is selected from aryl substituted with one or more halogen or —CF₃, or a 5 or 6 membered heterocycle.

11. A compound of claim 10 where R₄ is selected from pyrrolidine, oxane, pyridine, thiazole or pryazole.

12. A compound of claim 1 where R₅ is selected from aryl substituted with one or more halogens, —CF₃, or a heteroaryl.

13. A compound of claim 12 where R₅ is pyridine.

14. A compound of claim 1 where R₃ is a substituent other than bromo.

15. A compound of claim 14 where R₄ is a substituent other than chloro.

16. A compound of claim 1, where the compound is of Formula II 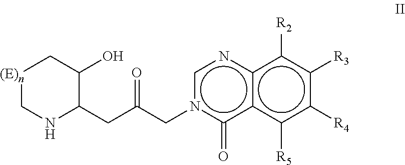
where:
E is CH₂, where n is 0 or 1;
R₂ is hydrogen, —CH₃ or —CH₂CH₃;
R₃ is hydrogen, —CH₃, or —OCH₃;
R₄ is selected from hydrogen, halogen, —CH₂CH₃, —CHCH₃CH₃, —OCF₃, —C≡N, substituted or unsubstituted aryl, substituted or unsubstituted heteroaryl, cycloalkyl, 5 or 6 membered substituted or unsubstituted saturated or unsaturated heterocycle, —SO₂CH₃, or —CF₃;
R₅ is selected from hydrogen, halogen, —CF₃, substituted or unsubstituted aryl, or substituted or unsubstituted heteroaryl, or cycloalkyl;
or a pharmaceutically acceptable salt thereof.

17. A compound of claim 1, where the compound is of Formula III 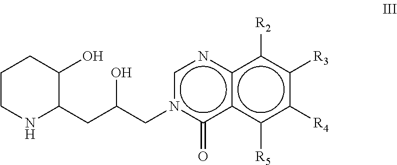
where:
R₂ is hydrogen, —CH₃ or —CH₂CH₃;
R₃ is hydrogen, —CH₃, or —OCH₃;
R₄ is selected from hydrogen, halogen, —CH₂CH₃, —CHCH₃CH₃, —OCF₃, —C≡N, substituted or unsubstituted aryl, substituted or unsubstituted heteroaryl, cycloalkyl, 5 or 6 membered substituted or unsubstituted saturated or unsaturated heterocycle, —SO₂CH₃, or —CF₃;
R₅ is selected from hydrogen, halogen, —CF₃, substituted or unsubstituted aryl, or substituted or unsubstituted heteroaryl, or cycloalkyl;
or a pharmaceutically acceptable salt thereof.

18. A compound of Formula IV 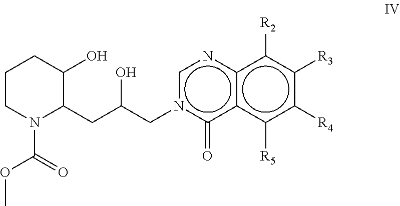
where:
R₂ is hydrogen, —CH₃ or —CH₂CH₃;
R₃ is hydrogen, —CH₃, or —OCH₃;
R₄ is selected from hydrogen, halogen, —CH₂CH₃, —CHCH₃CH₃, —OCF₃, —C≡N, substituted or unsubstituted aryl, substituted or unsubstituted heteroaryl, cycloalkyl, 5 or 6 membered substituted or unsubstituted saturated or unsaturated heterocycle, —SO₂CH₃, or —CF₃;
R₅ is selected from hydrogen, halogen, —CF₃, substituted or unsubstituted aryl, or substituted or unsubstituted heteroaryl, or cycloalkyl;
or a pharmaceutically acceptable salt thereof.

19. A compound of claim 18 where R₂ is —CH₃, R₃ is hydrogen; R₄ is halogen; and R₅ is hydrogen.

20. A compound of Formula V 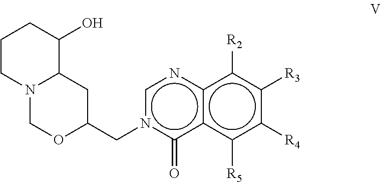
where:
R₂ is hydrogen, —CH₃ or —CH₂CH₃;
R₃ is hydrogen, —CH₃, or —OCH₃;
R₄ is selected from hydrogen, halogen, —CH₂CH₃, —CHCH₃CH₃, —OCF₃, —C≡N, substituted or unsubstituted aryl, substituted or unsubstituted heteroaryl, cycloalkyl, 5 or 6 membered substituted or unsubstituted saturated or unsaturated heterocycle, —SO₂CH₃, or —CF₃;
R₅ is selected from hydrogen, halogen, —CF₃, substituted or unsubstituted aryl, or substituted or unsubstituted heteroaryl, or cycloalkyl;
or a pharmaceutically acceptable salt thereof.

21. A compound of claim 20 where R₂ is —CH₃, R₃ is hydrogen; R₄ is halogen; and R₅ is hydrogen.

22. A compound of claim 1 selected from:
6-Chloro-3-{3-[(2R,3S)-3-hydroxypiperidin-2-yl]-2-oxopropyl}-8-methylquinazolin-4(3H)-one;
7-Chloro-3-{3-[(2R,3S)-3-hydroxypiperidin-2-yl]-2-oxopropyl}-8-methylquinazolin-4(3H)-one;
6-Bromo-3-{3-[(2R,3S)-3-hydroxypiperidin-2-yl]-2-oxopropyl}-8-methylquinazolin-4(3H)-one;
3-{3-[(2R,3S)-3-Hydroxypiperidin-2-yl]-2-oxopropyl}-7-methoxy-8-methylquinazolin-4(3H)-one;
5-Fluoro-3-{3-[(2R,3S)-3-hydroxypiperidin-2-yl]-2-oxopropyl}quinazolin-4(3H)-one;
6-Ethyl-3-(3-((2R,3S)-3-hydroxypiperidin-2-yl)-2-oxopropyl)-8-methylquinazolin-4(3H)-one;
3-{3-[(2R,3S)-3-Hydroxypiperidin-2-yl]-2-oxopropyl}-8-methyl-6-(trifluoromethoxy)quinazolin-4(3H)-one;
3-{3-[(2R,3S)-3-Hydroxypiperidin-2-yl]-2-oxopropyl}-8-methyl-6-(methylsulfonyl)quinazolin-4(3H)-one;
3-{3-[(2R,3S)-3-Hydroxypiperidin-2-yl]-2-oxopropyl}-8-methyl-6-(trifluoromethyl)quinazolin-4(3H)-one;
3-{3-[(2R,3S)-3-Hydroxypiperidin-2-yl]-2-oxopropyl}-6-isopropyl-8-methylquinazolin-4(3H)-one;
6-Cyclopropyl-3-{3-[(2R,3S)-3-hydroxypiperidin-2-yl]-2-oxopropyl}-8-methylquinazolin-4(3H)-one;
3-{3-[(2R,3S)-3-Hydroxypiperidin-2-yl]-2-oxopropyl}-8-methyl-4-oxo-3,4-dihydroquinazoline-6-carbonitrile;
6-Bromo-8-ethyl-3-{3-[(2R,3S)-3-hydroxypiperidin-2-yl]-2-oxopropyl}quinazolin-4(3H)-one;
8-Ethyl-6-fluoro-3-(3-((2R,3S)-3-hydroxypiperidin-2-yl)-2-oxopropyl)quinazolin-4(3H)-one;
5-(3-Fluorophenyl)-3-{3-[(2R,3S)-3-hydroxypiperidin-2-yl]-2-oxopropyl}quinazolin-4(3H)-one;
6-(3-Fluorophenyl)-3-{3-[(2R,3S)-3-hydroxypiperidin-2-yl]-2-oxopropyl}quinazolin-4(3H)-one;
6-(3-Chlorophenyl)-3-{3-[(2R,3S)-3-hydroxypiperidin-2-yl]-2-oxopropyl}quinazolin-4(3H)-one;
3-{3-[(2R,3S)-3-Hydroxypiperidin-2-yl]-2-oxopropyl}-6-[3-(trifluoromethyl)phenyl]quinazolin-4(3H)-one;
6-(3-Fluorophenyl)-3-{3-[(2R,3S)-3-hydroxypiperidin-2-yl]-2-oxopropyl}-8-methylquinazolin-4(3H)-one;
3-{3-[(2R,3S)-3-Hydroxypiperidin-2-yl]-2-oxopropyl}-8-methyl-6-[3-(trifluoromethyl)phenyl]quinazolin-4(3H)-one;
3-{3-[(2R,3S)-3-Hydroxypiperidin-2-yl]-2-oxopropyl}-8-methyl-6-(pyridin-3-yl)quinazolin-4(3H)-one;
6-(3,4-Difluorophenyl)-3-{3-[(2R,3S)-3-hydroxypiperidin-2-yl]-2-oxopropyl}-8-methylquinazolin-4(3H)-one;
3-{3-[(2R,3S)-3-Hydroxypiperidin-2-yl]-2-oxopropyl}-8-methyl-6-(tetrahydro-2H-pyran-4-yl)quinazolin-4(3H)-one;
3-{3-[(2R,3S)-3-Hydroxypiperidin-2-yl]-2-oxopropyl}-8-methyl-6-(pyrrolidin-1-yl)quinazolin-4(3H)-one;
3-{3-[(2R,3S)-3-Hydroxypiperidin-2-yl]-2-oxopropyl}-5-(trifluoromethyl)quinazolin-4(3H)-one;
3-{3-[(2R,3S)-3-Hydroxypiperidin-2-yl]-2-oxopropyl}-5-[3-(trifluoromethyl)phenyl]quinazolin-4(3H)-one;
5-(3,4-Difluorophenyl)-3-{3-[(2R,3S)-3-hydroxypiperidin-2-yl]-2-oxopropyl}quinazolin-4(3H)-one;
3-{3-[(2R,3S)-3-Hydroxypiperidin-2-yl]-2-oxopropyl}-5-(pyridin-3-yl)quinazolin-4(3H)-one; and
3-{3-[(2R,3S)-3-Hydroxypiperidin-2-yl]-2-oxopropyl}-8-methyl-5-(trifluoromethyl)quinazolin-4(3H)-one;
or salt thereof.

23. A compound of claim 1 selected from:
6-Chloro-3-{(S)-2-hydroxy-3-[(2R,3S)-3-hydroxypiperidin-2-yl]propyl}-8-methylquinazolin-4(3H)-one;
6-Chloro-3-{(S)-2-hydroxy-3-[(2R,3S)-3-hydroxypiperidin-2-yl]propyl}-7-methylquinazolin-4(3H)-one;
6-Fluoro-3-{(S)-2-hydroxy-3-[(2R,3S)-3-hydroxypiperidin-2-yl]propyl}-7-methylquinazolin-4(3H)-one;
6-Chloro-3-{(S)-2-hydroxy-3-[(2R,3S)-3-hydroxypiperidin-2-yl]propyl}-7-methoxyquinazolin-4(3H)-one;
6-Fluoro-3-{(S)-2-hydroxy-3-[(2R,3S)-3-hydroxypiperidin-2-yl]propyl}-8-methylquinazolin-4(3H)-one;
6-Chloro-3-{(S)-2-hydroxy-3-[(2R,3S)-3-hydroxypiperidin-2-yl]propyl}-8-(methyl-d3)quinazolin-4(3H)-one;
7-Chloro-3-{(S)-2-hydroxy-3-[(2R,3S)-3-hydroxypiperidin-2-yl]propyl}-8-methylquinazolin-4(3H)-one;
6-Bromo-3-{(S)-2-hydroxy-3-[(2R,3S)-3-hydroxypiperidin-2-yl]propyl}-8-methylquinazolin-4(3H)-one;
6-Chloro-8-ethyl-3-{(S)-2-hydroxy-3-[(2R,3S)-3-hydroxypiperidin-2-yl]propyl}quinazolin-4(3H)-one;
3-{(S)-2-Hydroxy-3-[(2R,3S)-3-hydroxypiperidin-2-yl]propyl}-6,8-dimethylquinazolin-4(3H)-one;
5-Fluoro-3-{(S)-2-hydroxy-3-[(2R,3S)-3-hydroxypiperidin-2-yl]propyl}quinazolin-4(3H)-one;
6-Ethyl-3-{(S)-2-hydroxy-3-[(2R,3S)-3-hydroxypiperidin-2-yl]propyl}-8-methylquinazolin-4(3H)-one;
3-{(S)-2-Hydroxy-3-[(2R,3S)-3-hydroxypiperidin-2-yl]propyl}-8-methyl-6-(trifluoromethoxy)quinazolin-4(3H)-one;
3-{(S)-2-Hydroxy-3-[(2R,3S)-3-hydroxypiperidin-2-yl]propyl}-8-methyl-6-(methylsulfonyl)quinazolin-4(3H)-one;
3-{(S)-2-Hydroxy-3-[(2R,3S)-3-hydroxypiperidin-2-yl]propyl}-8-methyl-6-(trifluoromethyl)quinazolin-4(3H)-one;
3-{(S)-2-Hydroxy-3-[(2R,3S)-3-hydroxypiperidin-2-yl]propyl}-6-isopropyl-8-methylquinazolin-4(3H)-one;
6-Cyclopropyl-3-{(S)-2-hydroxy-3-[(2R,3S)-3-hydroxypiperidin-2-yl]propyl}-8-methylquinazolin-4(3H)-one;
3-{(S)-2-Hydroxy-3-[(2R,3S)-3-hydroxypiperidin-2-yl]propyl}-8-methyl-4-oxo-3,4-dihydroquinazoline-6-carbonitrile;
6-Bromo-8-ethyl-3-{(S)-2-hydroxy-3-[(2R,3S)-3-hydroxypiperidin-2-yl]propyl}quinazolin-4(3H)-one;
5-(3-Fluorophenyl)-3-{(S)-2-hydroxy-3-[(2R,3S)-3-hydroxypiperidin-2-yl]propyl}quinazolin-4(3H)-one;
6-(3-Fluorophenyl)-3-{(S)-2-hydroxy-3-[(2R,3S)-3-hydroxypiperidin-2-yl]propyl}quinazolin-4(3H)-one;
6-(3-Chlorophenyl)-3-{(S)-2-hydroxy-3-[(2R,3S)-3-hydroxypiperidin-2-yl]propyl}quinazolin-4(3H)-one;
3-{(S)-2-hydroxy-3-[(2R,3S)-3-hydroxypiperidin-2-yl]propyl}-6-(3-(trifluoromethyl)phenyl)quinazolin-4(3H)-one;
6-(3-Fluorophenyl)-3-{(S)-2-hydroxy-3-[(2R,3S)-3-hydroxypiperidin-2-yl]propyl}-8-methylquinazolin-4(3H)-one;
3-{(S)-2-Hydroxy-3-[(2R,3S)-3-hydroxypiperidin-2-yl]propyl}-8-methyl-6-(3-(trifluoromethyl)phenyl)quinazolin-4(3H)-one;
3-{(S)-2-Hydroxy-3-[(2R,3S)-3-hydroxypiperidin-2-yl]propyl}-8-methyl-6-(pyridin-3-yl)quinazolin-4(3H)-one;
6-(3,4-Difluorophenyl)-3-{(S)-2-hydroxy-3-[(2R,3S)-3-hydroxypiperidin-2-yl]propyl}-8-methylquinazolin-4(3H)-one;
3-{(S)-2-Hydroxy-3-[(2R,3S)-3-hydroxypiperidin-2-yl]propyl}-8-methyl-6-(tetrahydro-2H-pyran-4-yl)quinazolin-4(3H)-one;
3-{(S)-2-Hydroxy-3-[(2R,3S)-3-hydroxypiperidin-2-yl]propyl}-8-methyl-6-(pyrrolidin-1-yl)quinazolin-4(3H)-one;
3-{(S)-2-Hydroxy-3-[(2R,3S)-3-hydroxypiperidin-2-yl]propyl}-5-(trifluoromethyl)quinazolin-4(3H)-one;
3-{(S)-2-Hydroxy-3-[(2R,3S)-3-hydroxypiperidin-2-yl]propyl}-5-[3-(trifluoromethyl)phenyl]quinazolin-4(3H)-one;
5-(3,4-Difluorophenyl)-3-{(S)-2-hydroxy-3-[(2R,3S)-3-hydroxypiperidin-2-yl]propyl}quinazolin-4(3H)-one;
3-{(S)-2-Hydroxy-3-[(2R,3S)-3-hydroxypiperidin-2-yl]propyl}-5-(pyridin-3-yl)quinazolin-4(3H)-one;
(2R,3S)-2-{(S)-3-[6-chloro-8-methyl-4-oxoquinazolin-3(4H)-yl]-2-hydroxypropyl}piperidin-3-ylpivalate;
3-{(S)-2-hydroxy-3-[(2R,3S)-3-hydroxypiperidin-2-yl]propyl}-8-methyl-6-(1-methyl-1H-pyrazol-3-yl)quinazolin-4(3H)-one;
Methyl (2R,3S)-2-((S)-3-(6-chloro-8-methyl-4-oxoquinazolin-3(4H)-yl)-2-hydroxypropyl)-3-hydroxypiperidine-1-carboxylate;
3-((S)-2-hydroxy-3-((2R,3S)-3-hydroxypiperidin-2-yl)propyl)-8-methyl-6-(thiazol-4-yl)quinazolin-4(3H)-one;
6-cyclobutyl-3-{(S)-2-hydroxy-3-[(2R,3S)-3-hydroxypiperidin-2-yl]propyl}-8-methylquinazolin-4(3H)-one;
5-Cyclopropyl-3-{(S)-2-hydroxy-3-[(2R,3S)-3-hydroxypiperidin-2-yl]propyl}quinazolin-4(3H)-one;
3-{(S)-2-hydroxy-3-[(2R,3S)-3-hydroxypiperidin-2-yl]propyl}-8-methyl-6-(piperidin-4-yl)quinazolin-4(3H)-one;
(2R,3S)-2-{(S)-3-[6-chloro-8-methyl-4-oxoquinazolin-3(4H)-yl]-2-hydroxypropyl}piperidin-3-ylpivalate;
3-{(S)-2-hydroxy-3-[(2R,3S)-3-hydroxypiperidin-2-yl]propyl}-8-methyl-6-(1-methyl-1H-pyrazol-3-yl)quinazolin-4(3H)-one; and
6-Chloro-3-{[(3S,4aR,5S)-5-hydroxyhexahydro-1H,3H-pyrido[1,2-c][1,3]oxazin-3-yl]methyl}-8-methylquinazolin-4(3H)-one;
or salt thereof.

24. A composition comprising a compound of claim 1, or a pharmaceutically acceptable salt thereof, and a pharmaceutically acceptable carrier.

25. A method of inhibiting glutamyl-prolyl tRNA synthetase comprising contacting glutamyl-prolyl tRNA synthetase with a compound of claim 1.

26. A method of treating a Th17-mediated condition comprising administering to a subject in need thereof a compound of claim 1.

27. A method of treating a disease selected from autoimmune diseases, dry eye syndrome, fibrosis, scar formation, angiogenesis, ischemic damage, inflammatory diseases, cancers, musculoskeletal diseases, cardiovascular diseases, transplant rejection, multiple sclerosis, systemic sclerosis and neurodegenerative diseases comprising administering to a subject in need thereof a compound of claim 1.
